(19) United States
(12) Patent Application Publication
(10) Pub. No.: US 2007/0154472 A1
(43) Pub. Date: Jul. 5, 2007

(54) SULFOTYROSINE SPECIFIC ANTIBODIES AND USES THEREFOR

(76) Inventors: Angela Widom, Acton, MA (US); Kimberly A. Marquette, Somerville, MA (US); Gray D. Shaw, Milton, MA (US); Louise A. Conroy, Cambridge (GB); David C. Lowe, Hertfordshire (GB)

Correspondence Address: WYETH/FINNEGAN HENDERSON, LLP, 901 NEW YORK AVENUE, NW, WASHINGTON, DC, 20001-4413, US

(21) Appl. No.: 11/608,564

(22) Filed: Feb. 20, 2007

Related U.S. Application Data
(60) Provisional application No. 60/748,927, filed on Dec. 9, 2005.

(51) Int. Cl. A61K 39/395; G01N 33/53; C07H 21/04; C12P 21/06; C12N 5/06; C07K 16/18

(52) U.S. Cl. 424/133.1; 435/7.1; 435/69.1; 435/320.1; 435/326; 530/388.1; 536/23.53

(57) ABSTRACT

This application relates to sulfotyrosine specific antibodies that are capable of binding selectively to sulfated tyrosine (sulfotyrosine), as well as their production and use. In certain embodiments, the antibodies distinguish sulfated tyrosine containing proteins from phosphorylated tyrosine containing proteins. Methods to detect or quantitate the presence of sulfotyrosine and/or sulfotyrosine containing protein in a biological sample, by adding a sulfotyrosine specific antibody to the sample are provided. Methods to treat systemic inflammatory response syndrome and sepsis by the administration of a sulfotyrosine specific antibody are also provided.

### RELATED APPLICATIONS

This application claims the benefit of U.S. Provisional Application No. 60/748,927, filed on Dec. 9, 2005, the contents of which are incorporated herein in their entirety by reference.

### BACKGROUND

Protein tyrosine sulfation is a widespread posttranslational modification that has been observed throughout the plant and metazoan animal kingdoms. While the carbohydrate moieties of glycoproteins may be sulfated, so far the only direct sulfation of proteins that has been identified occurs on tyrosine. Tyrosine sulfation is catalyzed by a family of enzymes known as tyrosylprotein sulfotransferases (TPSTs). TPSTs are trans-Golgi network (TGN) glycoproteins having their catalytic site oriented toward the lumen (type II orientation). Consequently, a selected subset of polypeptides that transit through the TGN of a cell may be sulfated This subset includes both secreted and membrane-bound polypeptides.

Analysis of known tyrosine sulfated peptides suggests TPSTs generally recognize acidic amino acid residues either adjacent or proximal to the tyrosine in the primary amino acid sequence of a substrate (Moore et al., J. Biol. Chem. 278:24243-46 (2003); Beisswanger et al., Proc. Natl. Acad. Sci. 95:11134-39 (1998)). The addition of the sulfate group (SO₄) on the tyrosine side chain increases the negative charge at that site, creating a sulfated tyrosine, or sulfotyrosine, residue, i.e., O-sulfo-L-tyrosine or 2-amino-3-(4-sulfooxyphenyl)-propanoic acid).

A diverse group of both receptor and ligand proteins contain tyrosine sulfation (Kehoe et al., Chem. Biol. 7:R57-61 (2000)), and tyrosine sulfation has been shown to enhance protein-protein interactions in multiple systems. For example, sulfation of one or more tyrosine residues in the N-terminal extracellular domain of CCR5, a major HIV co-receptor, is required for optiminal binding of MIP-1α/CCL3, MIP-1β/CCL4, and RANTES/CCL5 and for optimal HIV co-receptor function (Moore et al. J. Biol. Chem. 278:24243-46 (2003)). Further, hirudin sulfated at the tyrosine at position 63 (Tyr⁶³) has a 10-fold higher affinity for thrombin than unsulfated hirudin, and hirugen (N-acetylhirudin) binds α-thrombin through protein-protein hydrogen bonds involving the sulfato-oxygens of Tyr⁶³ (Id. at 24245). Also, sulfation of a tyrosine at position 1680 (Tyr¹⁶⁸⁰) in factor VIII is required for optimal binding to von Willebrand factor (vWF), and a tyrosine to phenylalanine substitution at that position is associated with mild to moderate hemophilia (Id.; Michnick et al., J. Biol. Chem. 269:20095-20102 (1994)).

Two examples of cell adhesion proteins with functionally important sulfated tyrosines are the P-selectin Glycoprotein Ligand 1 (PSGL-1) and platelet glycoprotein GPIbα. PSGL-1 is a leukocyte adhesion molecule that mediates cell tethering and rolling on activated endothelium cells under physiological blood flow. This activity is an important initial step in leukocyte extravasation. The mature amino terminus of PSGL-1 has an anionic segment with several sulfated tyrosines that is important for binding to P-selectin and L-selectin. The amino acid context of the sulfated tyrosines is substantially different in rat, mouse, and human PSGL-1, as the sulfated tyrosines are located within different primary amino acid sequences. High affinity interaction of PSGL-1 with P-selectin requires sulfation of tyrosines 46, 48, and 51 (human) or 54 and 56 (mouse) (Sako et al., Cell 83:323-331 (1995), Xia et al., Blood 101:552-559 (2003)). Platelet glycoprotein GPIbα mediates platelet tethering and rolling to immobilized vWF particularly under the forces of high shear blood flow. The sulfated tyrosines of human GPIbα at tyrosines 276, 278, and 279 are important for binding to both vWF and alpha thrombin (Dong et al., J. Biol. Chem. 276:16690-16694 (2001).

While radioactive isotope or high performance liquid chromatography (HPLC) has been used to assay levels of cellular sulfated tyrosine, these methods are not ideal. In radioisotope labeling experiments, the majority of ³⁵S is bound to the carbohydrate moieties of glycoproteins, making it difficult to identify the proteins containing sulfotyrosine, as it is estimated that only 0.3 to 4% of the ³⁵S radioactivity bound to proteins is incorporated as Tyr³⁵SO₃ (Liu et al., Proc. Natl. Acad. Sci. U.S.A. 82:7160-7164 (1985)).

Because sulfotyrosine is a component of secretory and membrane proteins in a variety of cells and tissues of many animals, prior attempts to identify sulfated tyrosine specific antibodies utilizing traditional immunization-based strategies were largely unsuccessful (but see, U.S. Pat. No. 5,716,836). Further, the similarity of phosphate-modified tyrosine to sulfate-modified tyrosine has been a problem for attempts to identify antibodies that specifically bind to sulfated tyrosine. Tyrosine O-sulfation, for example by sulfotransferases, is currently detected using cumbersome and inefficient radiolabeling techniques. Therefore, a need exists for antibodies capable of selectively binding to O-sulfated tyrosine to allow identification and purification of tyrosine-sulfated proteins, for example.

### SUMMARY

This application relates to sulfotyrosine specific antibodies that are capable of binding selectively to sulfated tyrosine, as well as their production and use.

In one aspect, the application provides an isolated antibody that specifically binds to sulfated tyrosine in a substantially context-independent manner. The antibody will bind a diverse set of polypeptides containing sulfated tyrosine, produced by either living cells or by synthetic chemical methods. In various embodiments the antibody specifically binds to sulfated tyrosine, but does not specifically bind to unsulfated tyrosine or phosphorylated tyrosine.

In other embodiments, the antibody comprises an amino acid sequence chosen from SEQ ID NO:2, SEQ ID NO:4, SEQ ID NO:6, SEQ ID NO:8, SEQ ID NO:10, and SEQ ID NO:12, wherein the antibody is capable of specifically binding to sulfated tyrosine in a substantially context-independent manner. Monoclonal, human, and scFv antibodies are specifically contemplated, as well as antibodies that specifically bind with an affinity constant greater than 10⁸ M⁻¹. In certain embodiments, the antibody specifically binds to an 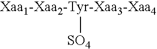
peptide as compared to the corresponding peptide having an unmodified or phosphorylated tyrosine residue, wherein Xaa₃ is not lysine. In some instances, Xaa₁, Xaa₂, Xaa₃, and/or Xaa₄ are optionally present in this epitope. In certain other embodiments, the antibody that specifically binds to sulfated tyrosine (denoted by lower case “y”), specifically binds to SEQ ID NO:25 (QATEyEyLDyDFL, a PSGL-1 peptide epitope) and SEQ ID NO:31 (DLyDyyPEED, a human GPIbα peptide epitope), but not SEQ ID NO:26 (QATEYEYLDYDFL, the non-sulfated PSGL-1 epitope).

Nonlimiting illustrative embodiments of the antibodies are referred to as PSG1 and PSG2. Other embodiments comprise a V_{H} and/or V_{L} domain of the Fv fragment of PSG1 or PSG2, or an scFv containing both the V_{H} and V_{L} domains (See, e.g., SEQ ID NOs:2, 4, 6, 8, 10, and 12). Further embodiments comprise one or more complementarity determining regions (CDRs) of any of these V_{H} and V_{L} domains (SEQ ID NOs:13-24). Other embodiments comprise an H3 fragment of the V_{H} domain of PSG1 or PSG2 (SEQ ID NO:15 or 21). Compositions comprising sulfotyrosine specific antibodies, and their use, are also provided.

In another aspect, the disclosure provides isolated nucleic acids, which comprise a sequence encoding an antibody described herein. Some embodiments include a nucleic acid comprising a nucleic acid that encodes a V_{H} or V_{L} domain from an Fv fragment of PSG1 or PSG2, or encodes an scFv containing both the V_{H} and V_{L} domains. Also provided are isolated nucleic acids, which comprise a sequence encoding one or more CDRs from any of the presently disclosed V_{H} and V_{L} domains, such as a sequence encoding an H3 CDR. The disclosure also provides DNA constructs and host cells comprising such nucleic acids.

The disclosure further provides a method of producing new V_{H} and V_{L} domains and/or functional antibodies comprising all or a portion of such domains derived from the V_{H} or V_{L} domains of PSG1 or PSG2.

In another aspect, the disclosure provides methods to identify and quantify proteins or peptides comprising sulfated tyrosine in a biological sample. In particular embodiments, the sulfotyrosine specific antibodies are used in a biomarker assay to detect proteins or peptides with sulfated tyrosine contained in a biological sample.

Additionally, sulfotyrosine specific antibodies may be used in diagnostic methods to detect sulfated proteins or peptides in a biological sample that are associated with a disease or disorder. The amount and distribution of sulfate modified tyrosine detected may be correlated with the expression and/or post-translational modification of a sulfated protein in the subject.

In another embodiment, sulfotyrosine specific antibodies are used for the treatment of sepsis in animals, including mammals such as humans.

It is to be understood that both the foregoing general description and the following detailed description are exemplary and explanatory only and are not restrictive of the claimed invention.

### BRIEF DESCRIPTION OF THE FIGURES

FIG. 1 shows the DNA sequence of PSG1 scFv (SEQ ID NO:1) in FIG. 1(A); the amino acid sequence of PSG1 scFv (SEQ ID NO:2) in FIG. 1(B), the V_{H} region in bold and the V_{L} region in bold underline; the amino acid sequence of the V_{H} region linked to a portion of human IgG4 in FIG. 1(C) (SEQ ID NO:335); and the V_{L} region linked to a portion of human lambda in FIG. 1(D) (SEQ ID NO:336). Variable region sequences are indicated in bold; the V_{H} region is shown in bold, and the V_{L} region is shown in bold underline in parts A and B.

FIG. 2 shows the DNA sequence of PSG2 scFv (SEQ ID NO:7) in FIG. 2(A); the amino acid sequence of PSG2 scFv (SEQ ID NO:8) in FIG. 2(B); the amino acid sequence of the V_{H} region linked to a portion of human IgG4 in FIG. 2(C) (SEQ ID NO:337); and the V_{L} region linked to a portion of human lambda in FIG. 2(D) (SEQ ID NO:338). As in FIG. 1, the variable region sequences are indicated in bold, with the V_{H} region shown in bold, and the V_{L} region shown in bold underline in parts A and B.

FIG. 3 shows the results of epitope mapping of the PSG2 antibody. FIG. 3(A) shows original epitope mapping of the PSG2 antibody, evaluating peptides that vary from the phagemid library panning peptide, as listed in Table 4. FIG. 3(B) shows a substitution analysis of a LDyDF (SEQ ID NO:28) peptide (where “y” is sulfated tyrosine and “Y” is non-sulfated tyrosine), and FIG. 3(C) shows a substitution analysis of a TEyER (SEQ ID NO:29) peptide. FIG. 3(D) shows binding of PSG2 to random peptides. The sequences of parts A and D are set forth in Table 4.

FIG. 4 shows the results of a BIAcore binding assay using bivalent forms of the PSG1 and PSG2 antibodies, indicating that PSG1 and PSG2 bind to a sulfated glycopeptide, 19.ek, derived from the sequence of PSGL-1 (SEQ ID NO:30, QATEyEyLDyDFLPETEPPRPMMDDDDK), but not to forms of the peptide without sulfate-modified tyrosine residues, regardless of whether an O-linked glycan is present. In contrast, the KPL-1 antibody specifically binds to the peptide, regardless of sulfation or glycosylation, thereby acting as a positive control. The 3D1 antibody is of a similar isotype to the PSG1 and PSG2 antibodies, binds an unrelated protein, and serves as a negative control.

FIG. 5(A) shows that in a dose-dependent fashion, mPSGL-1 Fc, a soluble murine PSGL-1 fusion protein containing the DPDyTyNTDP (SEQ ID NO:32) competitively inhibits the binding of PSG1 and PSG2 antibodies but not KPL-1 or PSL-275 antibodies to the biotinylated human PSGL1 peptide (bio-PSGL. 19.ek, SGP-3 form). FIG. 5(B) shows that in a dose-dependent fashion, GP1bα Fc, a soluble human GPIbα fusion protein containing the sequence DLyDyyPEED (SEQ ID NO:31) competitively inhibits the binding of PSG1 and PSG2 but not KPL-1 or PSL-275 antibodies with the biotinylated PSGL-1 peptide.

FIG. 6 shows that PSG2 is specific for the sulfotyrosine-containing GP1bα Fc fusion protein “GPG” and that it does not specifically bind to a phosphotyrosine-containing peptide, Phospho-BTK. In contrast, the anti-phosphotyrosine specific antibody (P-Tyr-100) specifically binds to the Phospho-BTK peptide, but not to the sulfotyrosine-containing GPG fusion protein. Neither antibody binds to the non-phosphorylated BTK peptide (BTK).

### DETAILED DESCRIPTION

The antibodies of this invention are capable of binding sulfate-modified tyrosine without a stringent amino acid context requirement. Sulfated tyrosine specific antibodies described herein bind specifically to multiple proteins or peptides that comprise a sulfated tyrosine residue. In certain embodiments, the antibodies distinguish sulfated tyrosine containing proteins from phosphorylated tyrosine containing proteins. These novel antibodies can be used to detect or quantitate the presence of sulfated tyrosine and/or sulfated tyrosine containing proteins, for example. In addition, the antibodies can be used to study the functional significance of a sulfated tyrosine within a polypeptide. Thus, the antibodies provide a useful tool for the study of protein tyrosine sulfation in vivo and in vitro.

In order that the present invention may be more readily understood, certain terms are first defined. Additional definitions are set forth throughout the detailed description.

I. Definitions

“Affinity tag,” as used herein, means a molecule attached to a second molecule of interest, capable of interacting with a specific binding partner for the purpose of isolating or identifying the second molecule of interest.

The term “antibody,” as used herein, refers to immunoglobulin molecules and immunologically active portions of immunoglobulin molecules, i.e., molecules that contain an antigen binding site that specifically binds (immunoreacts with) an antigen, such as a sulfated tyrosine or a polypeptide comprising a sulfated tyrosine. The term antibody encompasses any polypeptide comprising an antigen-binding site of an immunoglobulin regardless of the source, species of origin, method of production, and characteristics. As a non-limiting example, the term “antibody” includes human, orangutan, monkey, mouse, rat, goat, sheep, and chicken antibodies. The term includes but is not limited to polyclonal, monoclonal, human, humanized, single-chain, chimeric, synthetic, recombinant, hybrid, mutated, resurfaced, and CDR-grafted antibodies. For the purposes of the present invention, it also includes, unless otherwise stated, antibody fragments such as Fab, Fab′)₂, Fv, scFv, Fd, dAb, and other antibody fragments that retain the antigen-binding function. A “monoclonal antibody,” as used herein, refers to a population of antibody molecules that contain a particular antigen binding site and are capable of specifically binding to a particular epitope.

Antibodies can be made, for example, via traditional hybridoma techniques (Kohler et al., Nature 256:495-499 (1975)), recombinant DNA methods (U.S. Pat. No. 4,816,567), or phage display techniques using antibody libraries (Clackson et al., Nature 352:624-628 (1991); Marks et al., J. Mol. Biol. 222:581-597 (1991)). For various other antibody production techniques, see Antibody Engineering, 2^{nd} ed., Borrebaeck, Ed., Oxford University Press, 1995; Antibodies: A Laboratory Manual, Harlow et al., Eds., Cold Spring Harbor Laboratory, 1988. An antibody may comprise a heterologous sequence such as an affinity tag, for example.

The term “antigen-binding domain” refers to the part of an antibody molecule that comprises the area specifically binding to or complementary to a part or all of an antigen. Where an antigen is large, for example, an antibody may only bind to a particular part of the antigen. The “epitope” or “antigenic determinant” is a portion of an antigen molecule that is responsible for specific interactions with the antigen-binding domain of an antibody. An antigen-binding domain may be provided by one or more antibody variable domains (e.g., a so-called Fd antibody fragment consisting of a V_{H} domain). An antigen-binding domain comprises an antibody light chain variable region (V_{L}) and an antibody heavy chain variable region (V_{H}).

A “biological sample” is biological material collected from cells, tissues, organs, or organisms. Exemplary biological samples include serum, blood, plasma, biopsy sample, tissue sample, cell suspension, biological fluid, saliva, oral fluid, cerebrospinal fluid, amniotic fluid, milk, colostrum, mammary gland secretion, lymph, urine, sweat, lacrimal fluid, gastric fluid, synovial fluid, mucus, and other samples and clinical specimens.

The term “DNA construct,” as used herein, means a DNA molecule, or a clone of such a molecule, either single- or double-stranded that has been modified to contain segments of DNA combined in a manner that as a whole would not otherwise exist in nature. DNA constructs contain the information necessary to direct the expression of polypeptides of interest. DNA constructs can include promoters, enhancers and transcription terminators. DNA constructs containing the information necessary to direct the secretion of a polypeptide will also contain at least one secretory signal sequence.

The term “effective dose,” or “effective amount,” refers to a dosage or level that is sufficient to ameliorate clinical symptoms of, or achieve a desired biological outcome (e.g., decreased coagulation, increased fibrinolytic activity, reduction in a systemic inflammatory response, or increased organ function) in individuals, including individuals having systemic inflammatory response syndrome, sepsis, or septic shock. Such amount should be sufficient to reduce one or more clinical manifestations of the disorder. Therapeutic outcomes and clinical symptoms may include, for example, decreased coagulation, a decreased leukocyte count, or a reduction in one or more symptoms of a systemic inflammatory response such as, e.g., fever, delirium, chills, shaking, hypothermia, hyperventilation, or a rapid heartbbeat. In one embodiment, a sulfotyrosine specific antibody reduces clinical manifestations of a sepsis associated disorder. A sulfotyrosine specific antibody can cause a decrease in measured levels of pro-inflammatory cytokines and/or other markers of sepsis, for example. The effective amount can be determined as described in the subsequent sections. A “therapeutically effective amount” of a sulfotyrosine specific antibody refers to an amount which is effective, upon single or multiple dose administration to an individual (such as a human) at treating, preventing, curing, delaying, reducing the severity of, or ameliorating at least one symptom of a disorder or recurring disorder, or prolonging the survival of the subject beyond that expected in the absence of such treatment.

A “fragment,” as used herein, refers to a portion of a polypeptide or nucleic acid, such as a sequence of at least 5 contiguous residues, of at least 10 contiguous residues, of at least 15 contiguous residues, of at least 20 contiguous residues, of at least 25 contiguous residues, of at least 40 contiguous residues, of at least 50 contiguous residues, of at least 100 contiguous residues, or of at least 200 contiguous residues, that retains activity of the original protein. Fragments with a length of approximately 5, 10, 15, 20, 25, 30, 40, 50, 100, 200 residues, or more are contemplated, for example.

A protein or peptide “homolog,” as used herein, means that a relevant amino acid sequence of a protein or a peptide is at least 70%, 75%, 80%, 85%, 90%, 95%, 97%, 98%, 99%, or 100% identical to a given sequence. By way of example, such sequences may be variants derived from various species, or the homologous sequence may be recombinantly produced. The sequence may be derived from the given sequence by truncation, deletion, amino acid substitution or addition. Percent identity between two amino acid sequences is determined by standard alignment algorithms such as, for example, Basic Local Alignment Tool (BLAST) described in Altschul et al., J. Mol. Biol. 215:403-410 (1990). See also the algorithm of Needleman et al., J. Mol. Biol. 48:444-453 (1970); the algorithm of Meyers et al., Comput Appl. Biosci. 4:11-17 (1988); or Tatusova et al., FEMS Microbiol. Lett. 174:247-250 (1999), and other alignment algorithms and methods of the art.

The term “individual” refers to any vertebrate animal, including a mammal, bird, reptile, amphibian, or fish. The term “mammal” includes any animal classified as such, male or female, including humans, non-human primates, monkeys, dogs, horses, cats, rats, mice, guinea pigs, etc. Examples of non-mammalian animals include frog, chicken, turkey, duck, goose, fish, salmon, catfish, bass, and trout.

The term “isolated” refers to a molecule that is substantially free of its natural environment. For instance, an isolated protein is substantially free of cellular material or other proteins from the cell or tissue source from which it was derived. The term also refers to preparations where the isolated protein is at least 70-80% (w/w) pure; or at least 80-90% (w/w) pure; or at least 90-95% pure; or at least 80%, 85%, 90%, 95%, 96%, 97%, 98%, 99%, or 100% (w/w) pure. In some embodiments, the isolated molecule is sufficiently pure for pharmaceutical compositions.

“Linked,” as used herein, refers to a first nucleic acid sequence covalently joined to a second nucleic acid sequence. The first nucleic acid sequence can be directly joined or juxtaposed to the second nucleic acid sequence or alternatively an intervening sequence can covalently join the first sequence to the second sequence. Linked as used herein can also refer to a first amino acid sequence covalently joined to a second amino acid sequence. The first amino acid sequence can be directly joined or juxtaposed to the second amino acid sequence or alternatively an intervening sequence can covalently join the first amino acid sequence to the second amino acid sequence.

The term “reaction vessel” refers to a container in which an association of a molecule with an antibody that specifically binds to sulfated tyrosine can occur and be detected. A “surface” is the outer part of any solid (such as, e.g., glass, cellulose, polyacrylamide, nylon, polystyrene, polyvinyl chloride, dextran sulfate, or treated polypropylene) to which an antibody can be directly or indirectly “contacted,” “immobilized,” or “coated.” A “surface of a reaction vessel” may be a part of the vessel itself, or the surface may be in the reaction vessel. A surface such as polystyrene, for example, may be subjected to chemical or radiation treatment to change the binding properties of its surface. Low binding, medium binding, high binding, aminated, and activated surfaces are encompassed by the term. An antibody can be directly contacted with a surface, e.g., by physical adsorption or a covalent bond to the surface, or it can be indirectly contacted, e.g., through an interaction with a substance or moiety that is directly contacted with the surface.

The term “repertoire” refers to a genetically diverse collection of nucleotide sequences derived wholly or partially from sequences encoding immunoglobulins. The sequences may be generated by rearrangement in vivo of the V, D, and J segments of heavy chains, and the V and J segments of light chains. Alternatively, the sequences can be generated from a cell in response to which rearrangement occurs, e.g., in vitro stimulation. Alternatively, part or all of the sequences may be obtained by DNA splicing, nucleotide synthesis, mutagenesis, and other methods, see, e.g., U.S. Pat. No. 5,565,332.

The term “specific interaction,” or “specifically binds,” or the like, means that two molecules form a complex that is relatively stable under physiologic conditions. The term is also applicable where, e.g., an antigen-binding domain is specific for a particular epitope, which is found on a number of molecules. Thus, an antibody may specifically bind multiple proteins when it binds to an epitope present in each. For example polypeptides comprising a sulfated tyrosine residue may specifically bind to an antibody that recognizes a sulfated tyrosine as all or part of the epitope recognized by the antibody.

Specific binding is characterized by a selective interaction, often including high affinity binding with a low to moderate capacity. Nonspecific binding usually is a less selective interaction, and may have a low affinity with a moderate to high capacity. Typically, binding is considered specific when the affinity is at least 10⁶ M⁻¹, or preferably at least 10⁷ M⁻¹ or 10⁸ M⁻¹. An antibody does not specifically bind to a molecule if the level of measured binding is not substantially above background or non-specific binding levels. If necessary, non-specific binding can be reduced without substantially affecting specific binding by varying the binding conditions. Such conditions are known in the art, and a skilled artisan using routine techniques can select appropriate conditions. The conditions are usually defined in terms of concentration of antibodies, ionic strength of the solution, temperature, time allowed for binding, concentration of non-related molecules (e.g., serum albumin, milk casein), etc. Exemplary conditions are set forth in the Examples.

The phrase “substantially as set out” means that the relevant CDR, V_{H}, or V_{L} domain will be either identical or highly similar to the specified regions of which the sequence is set out herein. For example, such substitutions include 1 or 2 substitutes, additions, or deletions for every approximately 5 amino acids in the sequence of a CDR (H1, H2, H3, L1, L2, or L3). A sequence is “substantially identical” if it has no more than 1 nucleic acid or amino acid residue substituted, deleted, or added for every 10-20 residues in the sequence.

The phrase “substantially context-independent,” as used herein, refers to the conformation, sequence, or structure surrounding an antigenic determinant, such as a sulfated tyrosine residue. In the context of an epitope within a peptide or a protein, binding in a context-independent manner means binding to an epitope regardless of the surrounding amino acid sequence. To bind in a substantially context-independent manner, the antibody recognizes the sulfated tyrosine largely independent of specific amino acids adjacent or near the sulfated tyrosine residue.

The term “sulfated tyrosine” or “sulfotyrosine,” is used to include tyrosine-O-sulfate residues comprising a sulfate group covalently bound via the hydroxyl group of the tyrosine side chain. Alternatively, tyrosine may be O-sulfated at a terminal carboxyl group. A sulfated tyrosine may be free in solution, or it may be part of a molecule such as a peptide, protein, or other molecule. Sulfate may be added to a tyrosine by post-translational modification of a peptide or protein, by incorporation of an optionally protected sulfotyrosine building block during peptide synthesis, by chemical synthesis, or by chemical alteration, for example. As used herein, “Y” indicates a tyrosine residue, while “y” indicates a sulfated tyrosine.

II. Sulfotyrosine Specific Antibodies

The invention relates generally to antibodies that bind an epitope that includes a sulfated tyrosine, in which sulfated tyrosine is recognized free or in a variety of amino acid sequence contexts. The antibodies generally recognize tyrosine sulfated at the hydroxyl group of the tyrosine side chain. In one embodiment, the epitope consists of a sulfated tyrosine residue. In another embodiment, the epitope comprises a sulfated tyrosine in a peptide sequence, and the antibody recognizes the sulfated tyrosine largely independent of the sequence context. For example, the antibody may recognize an epitope comprising a sulfated-tyrosine at an internal position within an amino acid sequence and/or at the carboxy- or amino-terminus of an amino acid sequence. In yet another embodiment, the epitope comprises a sulfated tyrosine in an acidic peptide, or an acidic portion of a peptide (see also U.S. Patent Publication No. 2004/0002450). The disclosure also provides sulfotyrosine specific antibodies that comprise novel antigen-binding fragments.

The invention also relates generally to methods of making antibodies that bind to an epitope comprising a sulfated tyrosine, the method comprising transfecting a cell with a DNA construct, the construct comprising a DNA sequence encoding at least a portion of the anti-sulfotryosine antibodies of the invention, culturing the cell under conditions such that the antibody protein is expressed by the cell, and isolating the antibody protein.

In general, antibodies can be made, for example, using traditional hybridoma techniques (Kohler et al., Nature 256:495-499 (1975)), recombinant DNA methods (U.S. Pat. No. 4,816,567), or phage display performed with antibody libraries (Clackson et al., Nature 352:624-628 (1991); Marks et al., J. Mol. Biol. 222:581-597 (1991)). Antibodies are also produced recombinantly or synthetically. For other antibody production techniques, see also Antibodies: A Laboratory Manual, Harlow et al., Eds. Cold Spring Harbor Laboratory, 1988 or Antibody Engineering, 2^{nd} ed., Borrebaeck, Ed., Oxford University Press, 1995, for example. The antibodies are not limited to any particular source, species of origin, or method of production.

Intact antibodies, also known as immunoglobulins, are typically tetrameric glycosylated proteins composed of two light (L) chains of approximately 25 kDa each and two heavy (H) chains of approximately 50 kDa each. Two types of light chain, designated as the λ chain and the κ chain, are found in antibodies. Depending on the amino acid sequence of the constant domain of heavy chains, immunoglobulins can be assigned to five major classes: A, D, E, G, and M, and several of these may be further divided into subclasses (isotypes), e.g., IgG₁, IgG₂, IgG₃, IgG₄, IgA₁, and IgA₂.

The subunit structures and three-dimensional configurations of different classes of immunoglobulins are well known in the art. For a review of antibody structure, see Harlow et al., supra. Briefly, each light chain is composed of an N-terminal variable domain (V_{L}) and a constant domain (C_{L}). Each heavy chain is composed of an N-terminal variable domain (V_{H}), three or four constant domains (C_{H}), and a hinge region. The C_{H} domain most proximal to V_{H} is designated as C_{H}1. The V_{H} and V_{L} domains consist of four regions of relatively conserved sequence called framework regions (FR1, FR2, FR3, and FR4), which form a scaffold for three regions of hypervariable sequence called complementarity determining regions (CDRs). The CDRs contain most of the residues responsible for specific interactions with the antigen. The three CDRs are referred to as CDR1, CDR2, and CDR3. CDR constituents on the heavy chain are referred to as H1, H2, and H3, while CDR constituents on the light chain are referred to as L1, L2, and L3, accordingly. CDR3 and, particularly H3, are the greatest source of molecular diversity within the antigen-binding domain. H3, for example, can be as short as two amino acid residues or greater than 26.

The Fab fragment (Fragment antigen-binding) consists of the V_{H}-C_{H}1 and V_{L}-C_{L} domains covalently linked by a disulfide bond between the constant regions. To overcome the tendency of non-covalently linked V_{H} and V_{L} domains in the Fv to dissociate when co-expressed in a host cell, a so-called single chain (sc) Fv fragment (scFv) can be constructed. In a scFv, a flexible and adequately long linker connects either the C-terminus of the V_{H} to the N-terminus of the V_{L} or the C-terminus of the V_{L} to the N-terminus of the V_{H}. Most commonly, a 15-residue (Gly₄Ser)₃ peptide (SEQ ID NO:340) is used as a linker but other linkers are also known in the art.

The disclosure provides novel CDRs and variable regions derived from human immunoglobulin gene libraries. The structure for carrying a CDR, for example, will generally be an antibody heavy or light chain or a portion thereof, in which the CDR is located at a location corresponding to the CDR of naturally occurring V_{H} and V_{L}. The structures and locations of immunoglobulin variable domains may be determined, for example, as described in Kabat et al., Sequences of Proteins of Immunological Interest, No. 91-3242, National Institutes of Health Publications, Bethesda, Md., 1991.

DNA and amino acid sequences of sulfotyrosine specific antibodies, their scFv fragments, V_{H} and V_{L} domains, and CDRs are set forth in the Sequence Listing and are enumerated as listed in Table 1. Particular nonlimiting illustrative embodiments of the antibodies are referred to as PSG1 and PSG2. The CDR regions within the V_{H} and V_{L} domains of the illustrative embodiments are also listed in Table 1.

TABLE 1

| | | |
|---|---|---|
| Sequence | PSG1 | PSG2 |

| | | |
|---|---|---|
| scFv DNA | SEQ ID NO:1 | SEQ ID NO:7 |
| scFv AA | SEQ ID NO:2 | SEQ ID NO:8 |
| V_{H DNA} | SEQ ID NO:3 | SEQ ID NO:9 |
| V_{H AA} | SEQ ID NO:4 | SEQ ID NO:10 |
| V_{L DNA} | SEQ ID NO:5 | SEQ ID NO:11 |
| V_{L AA} | SEQ ID NO:6 | SEQ ID NO:12 |
| H1 AA | SEQ ID NO:13 | SEQ ID NO:19 |
| | AYYMH | SYGMT |
| H2 AA | SEQ ID NO:14 | SEQ ID NO:20 |
| | WINPNSGGTNYAQKFQG | SISSAGKTFYADSVKG |
| H3 AA | SEQ ID NO:15 | SEQ ID NO:21 |
| | GGPRVSSRPGIGYSDS | GRGHSYGRPLAS |
| L1 AA | SEQ ID NO:16 | SEQ ID NO:22 |
| | ASRIGAVTSGHYAN | TLRSGIDVGPHRIY |
| L2 AA | SEQ ID NO:17 | SEQ ID NO:23 |
| | RTNNKQS | KSDSDTQQGS |
| L3 AA | SEQ ID NO:18 | SEQ ID NO:24 |
| | LLYYGGSWV | MIWHSSAWV |

Sulfotyrosine specific antibodies may optionally comprise antibody constant regions or parts thereof. For example, a V_{L} domain may have attached, at its C terminus, antibody light chain constant domains including human Cκ or Cλ chains. Similarly, a specific antigen-binding domain based on a V_{H}domain may have attached all or part of an immunoglobulin heavy chain derived from any antibody isotope, e.g., IgG, IgA, IgE, and IgM and any of the isotope sub-classes, which include but are not limited to, IgG1 and IgG4. In the exemplary embodiments, PSG1 and PSG2 antibodies comprise C-terminal fragments of heavy chains of human IgG₄ (see, e.g., Thompson et al., J. Immunol. Methods. 227:17-29 (1999)) and light chains of human IgG_{1λ}. The DNA and amino acid sequences for the C-terminal fragments are well known in the art (see, e.g., Kabat et al., Sequences of Proteins of Immunological Interest, No. 91-3242, National Institutes of Health Publications, Bethesda, Md., 1991; Thompson et al., J. Immunol. Methods 227:17-29 (1999)).

TABLE 2

| | Amino acid |
|---|---|
| C-Terminal Region | Sequence |
| IgG1 heavy chain | SEQ ID NO: 33 |
| IgG4 heavy chain | SEQ ID NO: 34 |
| λ light chain | SEQ ID NO: 35 |
| κ light chain | SEQ ID NO: 36 |

The portion of an immunoglobulin constant region can be a portion of an immunoglobulin constant region obtained from any mammal. The portion of an immunoglobulin constant region includes a portion of a human immunoglobulin, a non-human primate immunoglobulin, a bovine immunoglobulin, a porcine immunoglobulin, a murine immunoglobulin, an ovine immunoglobulin or a rat immunoglobulin, for example.

The portion of an immunoglobulin constant region can include a portion of an IgG, an IgA, an IgM, an IgD, an IgE. In one embodiment, the immunoglobulin is an IgG. In another embodiment, the immunoglobulin is an IgG1. In yet another embodiment, the immunoglobulin is an IgG4.

The portion of an immunoglobulin constant region can include the entire heavy chain constant region, or a fragment or analog thereof. A heavy chain constant region can comprise a CH1 domain, a CH2 domain, a CH3 domain, and/or a hinge region, while a light chain constant region can comprise a CL domain. Thus, a constant region can comprise a CL, a CH1 domain, a CH2 domain, a CH3 domain, and/or a CH4 domain, for example.

The portion of an immunoglobulin constant region can include an Fc fragment. An Fc fragment can be comprised of the CH2 and CH3 domains of an immunoglobulin and the hinge region of the immunoglobulin. The Fc fragment can be the Fc fragment of an IgG1, an IgG2, an IgG3 or an IgG4. In one embodiment, the portion of an immunoglobulin constant region is an Fc fragment of an IgG1 or IgG4.

In another embodiment, specific IgG1 heavy chain, IgG4 heavy chain, λ light chain, and κ light chain sequences are the basis for the immunoglobulin constant region. For example, in some embodiments the portion of an immunoglobulin constant region comprises SEQ ID NOs:33, 34, 35, or 36 or an analog or fractional fragment thereof. In another embodiment, the portion of an immunoglobulin constant region consists of SEQ ID NO:33, 34, 35, or 36.

Certain embodiments comprise a V_{H} and/or V_{L} domain of an Fv fragment from PSG1 or PSG2, i.e. SEQ ID NOs:4, 6, 10, or 12. Further embodiments comprise at least one CDR of any of these V_{H} and V_{L} domains. Antibodies comprising at least one of the CDR sequences set out in SEQ ID NO:13-24 are encompassed within the scope of this invention. An embodiment, for example, comprises an H3 fragment of the V_{H} domain of antibodies chosen from at least one of PSG1 and PSG2, for example SEQ ID NOs:15 or 21.

In certain embodiments, the V_{H} and/or V_{L} domains may be germlined. For example, the framework regions (FRs) of these domains are mutated using molecular biology techniques to conform with those of the germline cells. A “germlined” sequence may be fully germlined or partially germlined, for example if some, but not all, variable domain residues conform with those of the germline cells. In other embodiments, the framework sequences remain diverged from the consensus germline sequences. In one embodiment, the invention provides amino acid and nucleic acid sequences for the germlined PSG1, PSG2, and/or antibodies comprising the amino acid sequences of Table 1, for example.

In an embodiment, mutagenesis is used to make an antibody more similar to one or more germline sequences. This may be desirable when mutations are introduced into the framework region of an antibody through somatic mutagenesis in the individuals whose antibody V genes were used to construct a phagemid library, such as the library described in Example 1, or through error prone PCR used to increase variability in the CDRs in a library. Germline sequences for the V_{H} and V_{L} domains can be identified by performing amino acid and nucleic acid sequence alignments against the VBASE database (MRC Center for Protein Engineering, UK). VBASE is a comprehensive directory of all human germline variable region sequences compiled from over a thousand published sequences, including those in the current releases of the Genbank and EMBL data libraries. In some embodiments, the FR regions of the scFvs are mutated in conformity with the closest matches in the VBASE database and the CDR portions are kept intact.

In certain embodiments, the antibodies specifically bind an epitope comprising a sulfated tyrosine in various amino acid sequence contexts. Preferably, the antibodies specifically bind to sulfotyrosine, but not to unsulfated tyrosine. In still other embodiments, the antibodies specifically bind to sulfated tyrosine in a substantially context-independent manner. In various embodiments the antibodies selectively bind to sulfotyrosine as compared to phosphotyrosine. In certain embodiments the antibodies specifically bind to sulfotyrosine, but not to phosphotyrosine. In some embodiments, the antibodies specifically bind a sulfotyrosine epitope with an affinity constant (Kₐ) of at least 10⁶ M⁻¹, 10⁷ M⁻¹, 10⁸ M⁻¹, 10⁹ M⁻¹ or 10¹⁰ M⁻¹. In some embodiments, the antibodies bind a corresponding non-sulfotyrosine epitope and/or a corresponding phosphotyrosine epitope with an affinity of less than 10² M⁻¹, 10³ M⁻¹, 10⁴ M⁻¹, or 10⁵ M⁻¹, for example.

In other embodiments, the antibodies specifically recognize sulfotyrosine in at least one protein, and/or free sulfotyrosine in solution. Antibodies described herein include antibodies that specifically bind to an epitope comprising sulfated tyrosine, such as part of a protein, a peptide, or free in solution. Further the antibodies may specifically bind to sulfated tyrosine that is naturally occurring or synthetic.

It is contemplated that antibodies of the invention may also bind with high affinity to some sulfotyrosine containing proteins, and yet with low to moderate affinity to sulfotyrosine in some other three-dimensional contexts. Epitope mapping (see, e.g., Epitope Mapping Protocols, Morris, Ed., Humana Press, 1996) and secondary and tertiary structure analyses can be carried out to identify specific 3D structures assumed by the disclosed antibodies and their complexes with antigens. Such methods include, but are not limited to, X-ray crystallography (Engstom, Biochem. Exp. Biol. 11:7-13(1974)) and computer modeling of virtual representations of the presently disclosed antibodies (Fletterick et al., Computer Graphics and Molecular Modeling, in Current Communications in Molecular Biology, Cold Spring Harbor Laboratory, Cold Spring Harbor, N.Y. (1986)).

Derivatives

This disclosure also provides a method for obtaining an antibody specific for sulfated tyrosine, such as an antibody that selectively binds to sulfated tyrosine as compared to phosphotyrosine. CDRs in such antibodies are not limited to the specific sequences of V_{H} and V_{L} identified in Table 1 and may include variants of these sequences that retain the ability to specifically bind sulfated tyrosine. Such variants may be derived from the sequences listed in Table 1 by a skilled artisan using techniques well known in the art. For example, amino acid substitutions, deletions, or additions, can be made in the FRs and/or in the CDRs. While changes in the FRs are usually designed to improve stability and immunogenicity of the antibody, changes in the CDRs are typically designed to increase affinity of the antibody for its target. Variants of FRs also include naturally occurring immunoglobulin allotypes. Such affinity-increasing changes may be determined empirically by routine techniques that involve altering the CDR and testing the affinity antibody for its target. For example, conservative amino acid substitutions can be made within any one of the disclosed CDRs. Various alterations can be made according to the methods described in Antibody Engineering, 2^{nd} ed., Borrebaeck, Ed., Oxford University Press, 1995. These include but are not limited to nucleotide sequences that are altered by the substitution of different codons that encode an identical or a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Substitutes for an amino acid within the sequence may be selected from other members of the class to which the amino acid belongs (see Table 3). Furthermore, any native residue in the polypeptide may also be substituted with alanine (see, e.g., MacLennan et al., Acta Physiol. Scand. Suppl. 643:55-67 (1998); Sasaki et al., Adv. Biophys. 35:1-24 (1998)).

Conservative modifications will produce molecules having functional and chemical characteristics similar to those of the molecule from which such modifications are made. In contrast, substantial modifications in the functional and/or chemical characteristics of the molecules may be accomplished by selecting substitutions in the amino acid sequence that differ significantly in their effect on maintaining (1) the structure of the molecular backbone in the area of the substitution, for example, as a sheet or helical conformation, (2) the charge or hydrophobicity of the molecule at the target site, or (3) the size of the molecule.

For example, a “conservative amino acid substitution” may involve a substitution of a native amino acid residue with a normative residue such that there is little or no effect on the polarity or charge of the amino acid residue at that position. (See, for example, MacLennan et al., Acta Physiol. Scand. Suppl. 643:55-67 (1998); Sasaki et al., Adv. Biophys. 35:1-24 (1998)). Exemplary substitutions are set forth in Table 3.

Desired amino acid substitutions (whether conservative or non-conservative) can be determined by those skilled in the art at the time such substitutions are desired. For example, amino acid substitutions can be used to identify important residues of the molecule sequence, or to increase or decrease the affinity of the molecules described herein.

Derivatives and analogs of antibodies of the invention can be produced by various techniques well known in the art, including recombinant and synthetic methods (Sambrook et al., Molecular Cloning: A Laboratory Manual, 2^{nd} ed., Cold Spring Harbor Laboratory Press (1989), and Bodansky et al., The Practice of Peptide Synthesis, 2^{nd} ed., Spring Verlag, Berlin, Germany (1995)).

TABLE 3

| Original | Exemplary | Typical |
|---|---|---|
| Residues | Substitutions | Substitutions |
| Ala (A) | Val, Leu, Ile, 2-Aminobutanoic Acid | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala, β-Alanine | Ala |
| His (H) | Asn, Gln, Lys, Arg | Arg |
| Ile (I) | Leu, Val, Met, Ala, Phe, Norleucine, | Leu |
| | Norvaline | |
| Leu (L) | Norleucine, Norvaline, Ile, Val, Met, Ala, Phe | Ile |
| Lys (K) | Arg, Ornithine, 1,4-Diaminobutyric Acid, | Arg |
| | 1,4-Diaminopropionic Acid, Gln, Asn | |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, Norleucine, Norvaline | Leu |

In one embodiment, a method for making a V_{H} domain which is an amino acid sequence variant of a V_{H} domain of the invention comprises a step of adding, deleting, substituting, or inserting one or more amino acids in the amino acid sequence of the presently disclosed V_{H} domain, optionally testing the V_{H} domain thus provided with one or more V_{L} domains, or testing the V_{H} domain separately or in a different combination. Antibodies, including immunoglobulin fragments, are optionally tested for specific binding to sulfated tyrosine, for binding to a sulfated tyrosine containing peptide or protein, or for binding to a negative control including an unmodified tyrosine and/or a phosphotyrosine residue. The ability of such antigen-binding domain to modulate the activity of a sulfotyrosine containing protein can also be tested. The V_{L} domain may have an amino acid sequence that is identical or is substantially as set out according to Table 1.

An analogous method can be employed in which one or more sequence variants of a V_{L} domain disclosed herein are combined with one or more V_{H} domains.

The antibodies described herein may be made by the procedures of Examples 1-2, and characterized by the assays of Examples 3-6, for example. A further aspect of the disclosure provides a method of preparing antigen-binding fragment that specifically binds with sulfated tyrosine. The method comprises:

(a) providing a starting repertoire of nucleic acids encoding a V_{H} domain that either includes a CDR3 to be replaced or lacks a CDR3 encoding region;

(b) combining the repertoire with a donor nucleic acid encoding an amino acid sequence substantially as set out herein for a V_{H} CDR3 (i.e., H3) such that the donor nucleic acid is inserted into the CDR3 region in the repertoire, so as to provide a product repertoire of nucleic acids encoding a V_{H} domain;

(c) expressing the nucleic acids of the product repertoire;

(d) selecting a binding fragment specific for sulfated tyrosine; and

(e) recovering the specific binding fragment or nucleic acid encoding it.

An analogous method may be employed in which a V_{L} CDR3 (i.e., L3) of the invention is combined with a repertoire of nucleic acids encoding a V_{L} domain, which either include a CDR3 to be replaced or lack a CDR3 encoding region. The donor nucleic acid for these methods may be selected from nucleic acids encoding an amino acid sequence substantially as set out in at least one of SEQ ID NOs:13-24.

A sequence encoding a CDR of the invention (e.g., CDR3) may be introduced into a repertoire of variable domains lacking the respective CDR (e.g., CDR3), using recombinant DNA technology, for example, using a methodology described by Marks et al., Bio/Technology 10:779-783 (1992). In particular, consensus primers directed at or adjacent to the 5′ end of the variable domain area can be used in conjunction with consensus primers to the third framework region of human V_{H} genes to provide a repertoire of V_{H} variable domains lacking a CDR3. The repertoire may be combined with a CDR3 of a particular antibody. Using analogous techniques, the CDR3-derived sequences may be shuffled with repertoires of V_{H} or V_{L} domains lacking a CDR3, and the shuffled complete V_{H} or V_{L} domains combined with a cognate V_{L} or V_{H} domain to make the sulfated tyrosine specific antibodies of the invention. The repertoire may then be displayed in a suitable host system such as the phage display system such as described in WO 92/01047 so that suitable antigen-binding fragments can be selected.

Analogous shuffling or combinatorial techniques are also disclosed by Stemmer, Nature 370:389-391 (1994), describing the technique in relation to a β-lactamase gene, but observing that the approach may be used for the generation of antibodies.

In further embodiments, one may generate novel V_{H} or V_{L} regions carrying one or more sequences derived from the sequences disclosed herein using random mutagenesis of one or more selected V_{H} and/or V_{L} genes. One such technique, error-prone PCR, is described in Gram et al., Proc. Natl. Acad. Sci. U.S.A. 89:3576-3580 (1992).

Another method that may be used is to direct mutagenesis to CDRs of V_{H} or V_{L} genes. Such techniques are disclosed in Barbas et al., Proc. Natl. Acad. Sci. U.S.A. 91:3809-3813 (1994) and Schier et al., J. Mol. Biol. 263:551-567 (1996).

Similarly, one or more, or all three, CDRs may be grafted into a repertoire of V_{H} or V_{L} domains, which are then screened for an antigen-binding fragment specific for sulfated tyrosine.

A portion of an immunoglobulin variable domain will comprise at least one of the CDRs substantially as set out herein and, optionally, intervening framework regions from the scFv fragments as set out herein. Residues at the N-terminal or C-terminal end of the variable domain may be heterologous, and may or may not be normally associated with naturally occurring variable domain regions. For example, construction of antibodies by recombinant DNA techniques may result in the introduction of N- or C-terminal residues encoded by linkers introduced to facilitate cloning or other manipulation steps. Other manipulation steps include the introduction of linkers to join variable domains to further protein sequences including immunoglobulin heavy chain constant regions, other variable domains (for example, in the production of diabodies), or proteinaceous labels as discussed in further detail below. Secretion signals or affinity tags are examples of heterologous sequences of certain embodiments of the antibodies provided herein.

Although the embodiments illustrated in the Examples comprise a “matching” pair of V_{H} and V_{L} domains, a skilled artisan will recognize that alternative embodiments may comprise antigen-binding fragments containing only a single CDR from either V_{L} or V_{H} domain or any combination of CDR sequences. Either of the single chain specific binding domains can be used to screen for complementary domains capable of forming a two-domain specific antigen-binding fragment capable of, for example, binding to sulfated tyrosine. The screening may be accomplished by phage display screening methods using the so-called hierarchical dual combinatorial approach disclosed in WO 92/01047, for example, in which an individual colony containing either an H or L chain clone is used to infect a complete library of clones encoding the other chain (L or H) and the resulting two-chain specific binding domain is selected in accordance with phage display techniques as described.

Anti-sulfotyrosine antibodies described herein can be linked to another functional and/or stabilizing molecule. For example, antibodies may be linked to another peptide or protein (albumin, another antibody, etc.), toxin, radioisotope, cytotoxic or cytostatic agents. The antibodies can be linked covalently by chemical cross-linking or by recombinant methods. The antibodies may also be linked to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337. The antibodies can be chemically modified by covalent conjugation to a polymer, for example, to increase their stability or half-life. Exemplary polymers and methods to attach them are also shown in U.S. Pat. Nos. 4,766,106; 4,179,337; 4,495,285; and 4,609,546.

The disclosed antibodies may also be altered to have a glycosylation pattern that differs from the native pattern. For example, one or more carbohydrate moieties can be deleted and/or one or more glycosylation sites added to the original antibody. Addition of glycosylation sites to the presently disclosed antibodies may be accomplished by altering the amino acid sequence to contain one or more glycosylation site consensus sequences known in the art. Another means of increasing the number of carbohydrate moieties on the antibodies is by chemical or enzymatic coupling of glycosides to the amino acid residues of the antibody. Such methods are described in WO 87/05330 and in Aplin et al., CRC Crit. Rev. Biochem. 22:259-306 (1981). Removal of any carbohydrate moieties from the antibodies may be accomplished chemically or enzymatically, for example, as described by Hakimuddin et al., Arch. Biochem. Biophys. 259:52 (1987); and Edge et al., Anal. Biochem. 118:131 (1981) and by Thotakura et al., Meth. Enzymol. 138:350 (1987).

The antibodies may also be tagged with a detectable label. A detectable label is a molecule which, by its chemical nature, provides an analytically identifiable signal which allows the detection of a molecular interaction. A protein, including an antibody, has a detectable label if it is covalently or non-covalently bound to a molecule that can be detected directly (e.g., by means of a chromophore, fluorophore, or radioisotope) or indirectly (e.g., by means of catalyzing a reaction producing a colored, luminescent, or fluorescent product). Detectable labels include a radiolabel such as ¹³¹I or ⁹⁹Tc, a heavy metal, or a fluorescent substrate, such as Europium, for example, which may also be attached to antibodies using conventional chemistry. Detectable labels also include enzyme labels such as horseradish peroxidase or alkaline phosphatase. Detectable labels further include chemical moieties such as biotin, which may be detected via binding to a specific cognate detectable moiety, e.g., labeled avidin.

Antibodies in which CDR sequences differ only insubstantially from those of the variable regions of SEQ ID NO:2, SEQ ID NO:4, SEQ ID NO:6, SEQ ID NO:8, SEQ ID NO:10, or SEQ ID NO:12 are encompassed within the scope of this invention. Typically, an amino acid is substituted by a related amino acid having similar charge, hydrophobic, or stereochemical characteristics. Such substitutions would be within the ordinary skills of an artisan. A skilled artisan would appreciate that changes can be made in FRs without adversely affecting the binding properties of an antibody. Changes to FRs include, but are not limited to, humanizing a non-human derived or engineering certain framework residues that are important for antigen contact or for stabilizing the binding site, e.g., changing the class or subclass of the constant region, changing specific amino acid residues which might alter the effector function such as Fc receptor binding, e.g., as described in U.S. Pat. Nos. 5,624,821 and 5,648,260 and Lund et al., J. Immunol. 147:2657-2662 (1991) and Morgan et al., Immunology 86:319-324 (1995), or changing the species from which the constant region is derived.

The skilled artisan will understand that portions of an immunoglobulin constant region for use in the antibody protein of the invention can include mutants or analogs thereof, or can include chemically modified immunoglobulin constant regions (e.g., pegylation) (see, e.g., Aslam and Dent 1998, Bioconjugation: Protein Coupling Techniques For the Biomedical Sciences Macmilan Reference, London) or fragments thereof.

One of skill in the art will appreciate that the modifications described above are representative only, and that many other modifications would be obvious to a skilled artisan in light of the teachings of the present disclosure.

III. Nucleic Acids, Cloning, and Expression Systems

The present disclosure further provides isolated nucleic acids encoding the disclosed antibodies. The nucleic acids may comprise DNA or RNA and may be wholly or partially synthetic or recombinant. Reference to a nucleotide sequence as set out herein encompasses a double or single stranded DNA molecule with the specified sequence, and encompasses an RNA molecule with the specified sequence in which U is substituted for T, unless context requires otherwise.

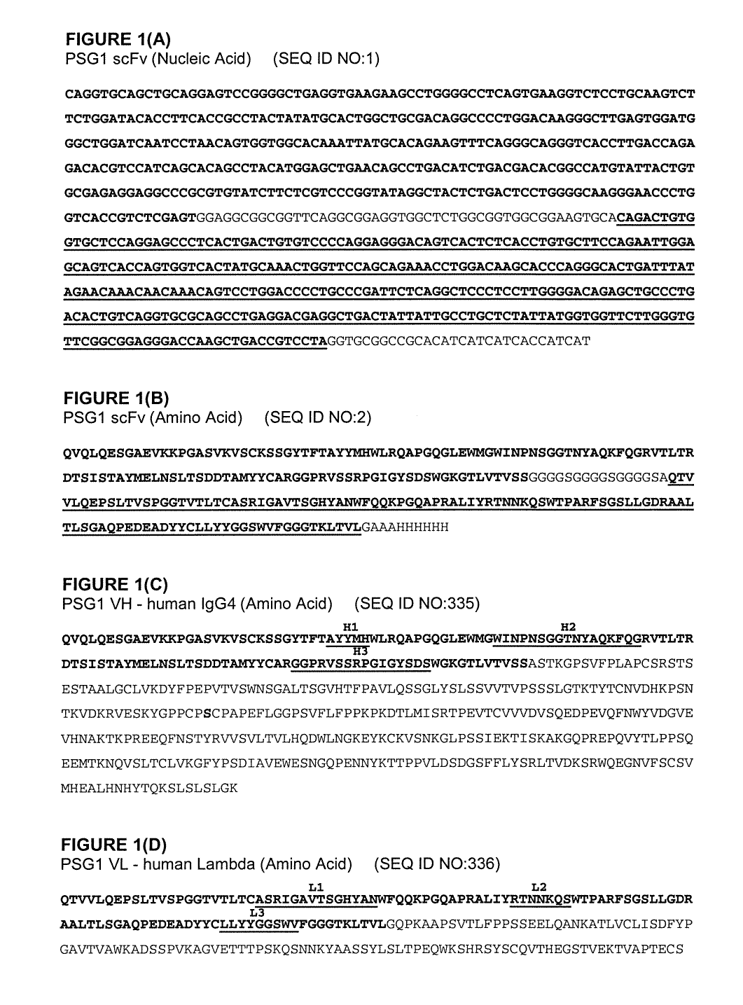

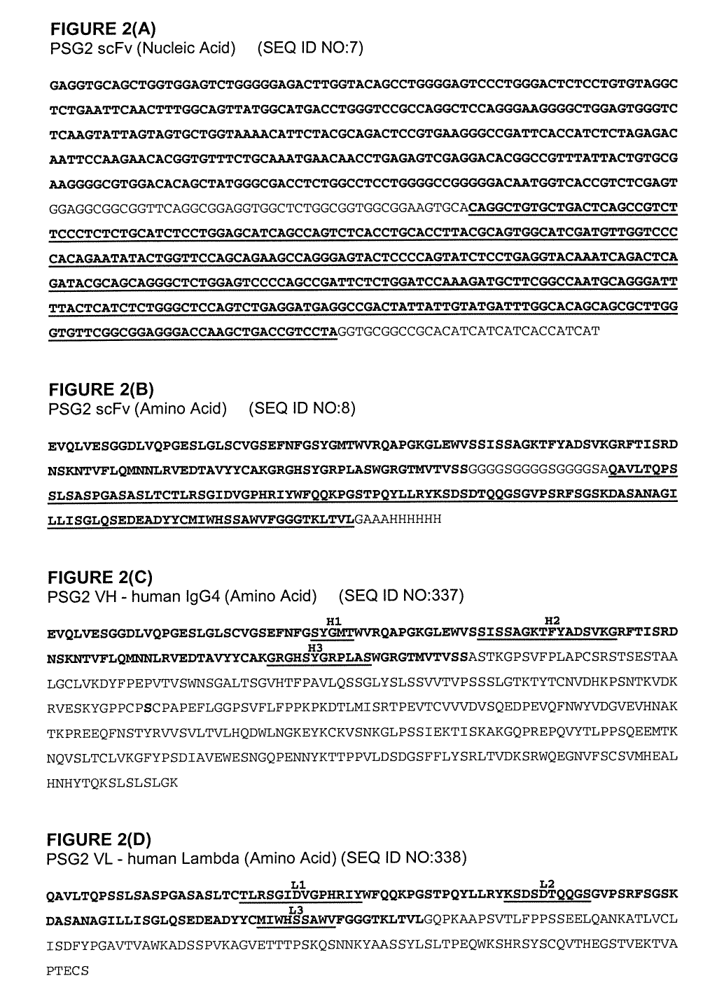

The nucleic acids provided herein comprise a coding sequence for a CDR, a V_{H} domain, and/or a V_{L} domain disclosed herein. Similarly, nucleic acid fragments encoding portions of these antibodies are disclosed. In one embodiment, the nucleic acid construct comprises the DNA sequence of FIG. 1A SEQ ID NO:1) or a homolog thereof. In another embodiment, the nucleic acid construct comprises the DNA sequence of FIG. 2A (SEQ ID NO:3) or an analog thereof. In another embodiment, the nucleic acid construct comprises a nucleic acid that encodes one or more antibody sequences set forth in the sequence listing.

The present disclosure also provides constructs in the form of plasmids, vectors, phagemids, transcription or expression cassettes which comprise at least one nucleic acid encoding a CDR, a V_{H} domain, and/or a V_{L} domain disclosed herein.

The disclosure further provides a host cell which comprises one or more constructs as above.

Also provided are nucleic acids encoding any CDR (H1, H2, H3, L1, L2, or L3), V_{H} or V_{L} domain, as well as methods of making the encoded products. The method comprises expressing the encoded product from the encoding nucleic acid. Production may be achieved by culturing recombinant host cells containing the nucleic acid under appropriate conditions. Following production, a V_{H} or V_{L} domain or other antibody or specific fragment may be isolated and/or purified using any suitable technique, then used as appropriate.

Antigen-binding fragments, V_{H} and/or V_{L} domains, and the nucleic acid molecules and vectors encoding the same may be isolated and/or purified from their natural environment, in substantially pure or homogeneous form, or, in the case of nucleic acid, free or substantially free of nucleic acid or other contaminating factors.

The invention also provides isolated DNA sequences encoding polypeptides of the invention that differ from a reference antibody sequence, but retain the antigen specificity. For example, variant sequences that encode a polypeptide that specifically binds to sulfated tyrosine, but not to phosphotyrosine and/or non-sulfated tyrosine are described herein. Due to the known degeneracy of the genetic code, wherein more than one codon can encode the same amino acid, a DNA sequence can vary from that shown in SEQ ID NOs:1 or 3 and still encode a polypeptide having the amino acid sequence of SEQ ID NOs:2 or 4, for example. Such variant DNA sequences can result from naturally occurring, accidental, and/or deliberate mutagenesis of a native sequence. A nucleic acid capable of hybridizing to a nucleic acid that encodes a sulfotyrosine specific antibody under high stringency conditions as well as a nucleic acid that differs from a nucleotide sequence, such as SEQ ID NOs:1, 3, 5, 7, 9, or 11 are also described herein.

In another embodiment, the nucleic acid molecules of the invention also comprise nucleotide sequences that are at least 80% identical or that encode an amino acid that is at least 80% identical to a native sequence. Also contemplated are embodiments in which a sequence is at least 85%, 90%, 95%, 96%, 97%, 98%, 99%, or 99.5% identical to a reference sequence. The percent identity may be determined by visual inspection and mathematical calculation. Alternatively, the percent identity of two nucleic acid sequences can be determined by comparing sequence information using the GAP computer program, version 6.0 described by Devereux et al., Nucl. Acids Res. 12:387 (1984) and available from the University of Wisconsin Genetics Computer Group (UWGCG).

Systems for cloning and expression of a polypeptide in a variety of different host cells are well known in the art. For cells suitable for producing antibodies, see Gene Expression Systems; Fernandez et al., Eds.; Academic Press, 1999. Briefly, suitable host cells include bacteria, yeast, insect, plant, animal, and mammalian cells, and yeast and baculovirus expression systems may be appropriate. Mammalian cell lines available in the art for expression of a heterologous polypeptide include Chinese hamster ovary cells, HeLa cells, baby hamster kidney cells, NSO mouse myeloma cells, and many others. A common bacterial host is E. coli. Any protein expression system compatible with the invention may be used to produce the disclosed antibodies. Suitable expression systems include transgenic animals described in Gene Expression Systems; Fernandez et al., Eds.; Academic Press, 1999.

Suitable vectors or DNA constructs can be chosen or constructed, so that they contain appropriate regulatory sequences, including promoter sequences, terminator sequences, polyadenylation sequences, enhancer sequences, marker or selection genes, and other sequences as appropriate. Constructs may be plasmids or viral, e.g., phage, or phagemid, as appropriate. In one embodiment, the nucleic acid construct is comprised of DNA. In another embodiment, the nucleic acid construct is comprised of RNA. The nucleic acid construct can be a vector, e.g., a viral vector or a plasmid. Examples of viral vectors include, but are not limited to, an adeno virus vector, an adeno-associated virus vector, or a murine leukemia virus vector. Examples of plasmids include, but are not limited to, pUC and pGEX. For further details see, for example, Sambrook et al., Molecular Cloning: A Laboratory Manual, 2^{nd} ed., Cold Spring Harbor Laboratory Press, 1989. Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, 2^{nd} ed., Ausubel et al., Eds., John Wiley & Sons, 1992.

A further aspect of the disclosure provides a host cell comprising a nucleic acid as disclosed here. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage, for example. The introduction of the nucleic acid into the cells may be followed by causing or allowing expression from the nucleic acid, e.g., by culturing host cells under conditions for expression of the gene.

IV. Production of Antibody Proteins

Antibody proteins of the invention can be produced using techniques well known in the art. For example, the antibody proteins of the invention can be produced recombinantly in cells (see, e.g., Sambrook et al., Molecular Cloning: A Laboratory Manual, Cold Spring Harbor Laboratory, N.Y., 1989; and Ausubel et al. Current Protocols in Molecular Biology, Greene Publishing Associates and Wiley Interscience, N.Y., 1989). Alternatively, the antibody proteins of the invention can be produced using known synthetic methods such as solid phase synthesis. Synthetic techniques are well known in the art (see, e.g., Merrifield, Chemical Polypeptides, Katsoyannis and Panayotis Eds., 1973, pp. 335-61; Merrifield, J. Am. Chem. Soc. 85:2149 (1963); Davis et al., Biochem. Intl. 10:394 (1985); Finn et al., The Proteins (3^{rd} ed.) 2:105 (1976); Erikson et al., The Proteins (2^{nd} ed.) 2:257 (1976); U.S. Pat. No. 3,941,763). Further, the antibody proteins of the invention can be produced using a combination of recombinant and synthetic methods. In certain applications, it may be beneficial to use either a recombinant method or a combination of recombinant and synthetic methods.

For recombinant production, a polynucleotide sequence encoding the antibody protein is inserted into an appropriate expression vehicle, such as a vector which contains the necessary elements for the transcription and translation of the inserted coding sequence, or in the case of an RNA viral vector, the necessary elements for replication and translation. The nucleic acid encoding the antibody protein is inserted into the vector in proper reading frame.

The expression vehicle is then transfected into a suitable target cell which will express the peptide. Transfection techniques known in the art include, but are not limited to, calcium phosphate precipitation (Wigler et al., Cell 14:725 (1978)) and electroporation (Neumann et al., EMBO J. 1:841 (1982)). A variety of host-expression vector systems may be utilized to express the antibody proteins described herein including both prokaryotic (e.g., E. coli) or eukaryotic cells. These include, but are not limited to, microorganisms such as bacteria (e.g., E. coli) transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems, including mammalian cells (e.g., CHO cells, Cos cells, HeLa cells, myeloma cells).

When the antibody protein is expressed in a eukaryotic cell, the DNA encoding the antibody protein may also code for a signal sequence that will permit the antibody protein to be secreted. One skilled in the art will understand that a signal sequence is translated and that it may be cleaved from the polypeptide to form the mature antibody protein. Various signal sequences are known in the art, e.g., the interferon α signal sequence and the mouse Igκ light chain signal sequence. Alternatively, where a signal sequence is not included the antibody protein can be recovered by lysing the cells.

When the antibody protein of the invention is recombinantly synthesized in a prokaryotic cell, it may be desirable to refold the protein. The antibody protein produced by this method can be refolded to a biologically active conformation using conditions known in the art, e.g., denaturing and reducing conditions and then slow dialysis in PBS.

Depending on the expression system used, the expressed peptide is then isolated by procedures well-established in the art (e.g., affinity chromatography, size exclusion chromatography, and/or ion exchange chromatography).

The expression vectors can encode an affinity tag to permit easy purification of the recombinantly produced protein. Examples include, but are not limited to, histidine tags, flag tags, and maltose protein binding tags. For example, vector pUR278 (Ruther et al., EMBO J. 2:1791 (1983)) may be used in which the coding sequence of the antibody of the invention may be ligated into the vector in frame with the lac z coding region so that a hybrid protein is produced. In another example, pGEX vectors may be used to express proteins with a glutathione S-transferase (GST) tag. GST fusion proteins are often soluble and can be purified from cells by adsorption to glutathione-agarose beads followed by elution in the presence of free glutathione. The vectors optionally include cleavage sites (thrombin or factor Xa protease or PreScission Protease™ (Pharmacia, Peapack, N.J.) for removal or cleavage of the tag after purification of the polypeptide.

Vectors used in transformation will usually contain a selectable marker used to identify transformants. In bacterial systems this can include an antibiotic resistance gene such as ampicillin or kanamycin. Selectable markers for use in cultured mammalian cells include genes that confer resistance to drugs, such as neomycin, hygromycin, and methotrexate. The selectable marker may be an amplifiable selectable marker. One amplifiable selectable marker is the DHFR gene. Another amplifiable marker is the DHFRr cDNA (Simonsen and Levinson, Proc. Natl. Acad. Sci. U.S.A. 80:2495 (1983)). Selectable markers are reviewed by Thilly (Mammalian Cell Technology, Butterworth Publishers, Stoneham, Mass.), and the choice of selectable markers is well within the level of ordinary skill in the art.

The expression elements of the expression systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used. When cloning in insect cell systems, promoters such as the baculovirus polyhedron promoter may be used. When cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used. When cloning in mammalian cell systems, promoters derived from the genome of mammalian cells (e.g., metallothionein promoter) or from mammalian viruses (e.g., the adenovirus late promoter; the vaccinia virus 7.5 K promoter; the CMV promoter) may be used. When generating cell lines that contain multiple copies of expression product, SV40-, BPV- and EBV-based vectors may be used with an appropriate selectable marker.

In cases where plant expression vectors are used, the expression of sequences encoding linear or non-cyclized forms of the antibody proteins of the invention may be driven by any of a number of promoters. For example, viral promoters such as the 35S RNA and 19S RNA promoters of CaMV (Brisson et al., Nature 310:511-514 (1984)), or the coat protein promoter of TMV (Takamatsu et al., EMBO J. 6:307-311 (1987)) may be used; alternatively, plant promoters such as the small subunit of RUBISCO (Coruzzi et al., EMBO J. 3:1671-1680 (1984); Broglie et al., Science 224:838-843 (1984)) or heat shock promoters, e.g., soybean hsp17.5-E or hsp17.3-B (Gurley et al., Mol. Cell. Biol. 6:559-565 (1986)) may be used. These constructs can be introduced into plant cells using Ti plasmids, Ri plasmids, plant virus vectors, direct DNA transformation, microinjection, electroporation, etc. For reviews of such techniques see, e.g., Weissbach & Weissbach 1988, Methods for Plant Molecular Biology, Academic Press, NY, Section VIII, pp. 421-463; and Grierson & Corey 1988, Plant Molecular Biology, 2d ed., Blackie, London, Ch. 7-9.

In one insect expression system that may be used to produce the antibody proteins of the invention, Autographa californica nuclear polyhidrosis virus (AcNPV) is used as a vector to express the foreign genes. The virus grows in Spodoptera frugiperda cells. A coding sequence for a heterologous polypeptide may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (see, e.g., Smith et al., J. Virol. 46:584 (1983); U.S. Pat. No. 4,215,051). Further examples of this expression system may be found in Ausubel et al., Eds. 1989, Current Protocols in Molecular Biology, Vol. 2, Greene Publish. Assoc. & Wiley Interscience.

In mammalian host cells, a number of expression systems may be utilized, such as viral-based systems. In cases where an adenovirus is used as an expression vector, a coding sequence may be ligated to an adenovirus transcription/translation control complex, e.g., the late promoter and tripartite leader sequence. This antibody gene may then be inserted in the adenovirus genome by in vitro or in vivo recombination.

In cases where an adenovirus is used as an expression vector, a coding sequence may be ligated to an adenovirus transcription/translation control complex, e.g., the late promoter and tripartite leader sequence. This antibody gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts (see, e.g., Logan et al., Proc. Natl. Acad. Sci. U.S.A. 81:3655-3659 (1984)). Alternatively, the vaccinia 7.5 K promoter may be used (see, e.g., Mackett et al., Proc. Natl. Acad. Sci. U.S.A. 79:7415-7419 (1982); Mackett et al., J. Virol. 49:857-864 (1984); Panicali et al., Proc. Natl. Acad. Sci. U.S.A. 79:4927(1982)).

Host cells containing DNA constructs of the antibody protein are grown in an appropriate growth medium. As used herein, the term “appropriate growth medium” means a medium containing nutrients required for the growth of cells. Nutrients required for cell growth may include a carbon source, a nitrogen source, essential amino acids, vitamins, minerals, and growth factors. Optionally, the media can contain bovine calf serum or fetal calf serum. The growth medium will generally select for cells containing the DNA construct by, for example, drug selection or deficiency in an essential nutrient which is complemented by the selectable marker on the DNA construct or co-transfected with the DNA construct. Cultured mammalian cells are generally grown in commercially available serum-containing or serum-free media (e.g., MEM, DMEM). Selection of a medium appropriate for the particular cell line used is within the level of ordinary skill in the art.

The recombinantly produced antibody protein of the invention can be isolated from culture media. The culture medium from appropriately grown transformed or transfected host cells is separated from the cell material, and the presence of antibody proteins is demonstrated. One method of detecting the antibody proteins, for example, is by the binding of the antibody proteins or portions of the antibody proteins to a specific antibody recognizing the antibody protein of the invention (e.g., an anti-Fc antibody). An anti-antibody protein antibody may be a monoclonal or polyclonal antibody raised against the antibody protein in question. For example, the antibody protein can contain a portion of an immunoglobulin constant region. Antibodies recognizing the constant region of many immunoglobulins are known in the art and are commercially available. An antibody can be used to perform an ELISA or a western blot to detect the presence of the antibody protein of the invention.

The antibody protein of the invention is optionally produced in a transgenic animal, such as a rodent. The term “transgenic animals” refers to non-human animals that have incorporated a foreign gene into their genome. Because this gene is present in germline tissues, it is passed from parent to offspring. Methods of producing transgenic animals are known in the art, including transgenics that produce immunoglobulin molecules (Wagner et al., Proc. Natl. Acad. Sci. U.S.A. 78:6376 (1981); McKnight et al., Cell 34:335 (1983); Brinster et al., Nature 306:332 (1983); Ritchie et al., Nature 312:517(1984)).

The invention also relates to a pharmaceutical composition comprising one or more anti-sulfotyrosine antibodies or active portions thereof and a pharmaceutically acceptable carrier or excipient. The compositions may also contain other active compounds providing supplemental, additional, or enhanced therapeutic functions. Examples of suitable pharmaceutical carriers are described in Remington's Pharmaceutical Sciences by E. W. Martin. Examples of excipients can include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like as well as those described infra. The composition optionally contains pH buffering reagents, and wetting or emulsifying agents. The pharmaceutical compositions may also be included in a container, pack, or dispenser together with instructions for administration.

The presently disclosed antibodies may be prepared with carriers that will protect the compound against rapid elimination from the body, such as a controlled release formulation, including implants and microencapsulated delivery systems.

V. Detection Methods

The antibodies of the present invention may be used to detect the presence of proteins comprising a sulfotyrosine residue, in vivo or in vitro. Such methods allow a detection of a disorder associated with tyrosine sulfate or sulfotyrosine, for example. Further, by correlating the presence or level of these proteins or of sulfotyrosine in these proteins with a medical condition, detection of the proteins comprising a sulfotyrosine detects or diagnoses the medical condition. Tyrosine sulfation has functional importance in leukocyte adhesion, hormone synthesis, chemokine receptor signaling, and hemostasis, for example (Önnerfjord et al., J. Biol. Chem. 279:26-33 (2004)). Detection of sulfotyrosine may be used to detect or diagnose disorders associated with these processes or with a protein comprising a sulfotyrosine, for example. Also, post-translational modification of proteins by tyrosine sulfation increases the affinity of extracellular ligand-receptor interactions important in the immune response as well as in other biological processes in animals. For example, sulfated tyrosines in polyomavirus and varicella-zoster virus may help modulate host cell recognition and facilitate viral attachment and entry (Lin et al., Biochem. Biophys. Res. Commun. 312:1154-58 (2003) (surveying predicted sites of tyrosine sulfation in 1024 viruses)).

Methods to Detect Sulfated Tyrosine

Methods to detect and/or quantify sulfotyrosine-containing molecules using the antibodies described herein are encompassed by this application. Detection methods and assays are well known in the art and include ELISA, radioimmunoassay, immunoblot, Western blot, immunofluorescence, immunoprecipitation, surface plasmon resonance, and other comparable techniques.

Where the antibodies are intended for detection or diagnostic purposes, it may be desirable to modify them, for example, with a ligand group (such as biotin) or a detectable marker group (such as a fluorescent group, a radioisotope or an enzyme). If desired, the antibodies (whether polyclonal or monoclonal) may be labeled using conventional techniques. Suitable labels include fluorophores, chromophores, radioactive atoms, electron-dense reagents, such as heavy metals, enzymes, and ligands having specific binding partners. Enzymes are typically detected by their activity. For example, horseradish peroxidase can be detected by its ability to convert tetramethylbenzidine (TMB) to a blue pigment, quantifiable with a spectrophotometer. Other suitable labels may include biotin and avidin or streptavidin, IgG and protein A, and the numerous receptor-ligand couples known in the art. Other permutations and possibilities will be readily apparent to those of ordinary skill in the art, and are considered as equivalents within the scope of the instant invention.

Proteins Comprising Sulfated Tyrosine

Methods to detect, quantitate, or purify proteins comprising a sulfotyrosine or molecules comprising sulfotyrosine are provided herein. Sulfated tyrosine, sulfated tyrosine in various amino acid sequence contexts, and sulfated tyrosine in a protein context are detected by antibodies and methods provided herein. Various naturally occurring proteins comprise a sulfated tyrosine, which is added by post-translational modification of a polypeptide during its transit through the trans-Golgi network. For example, the location of sulfation on several proteins has been defined and is well known in the art for certain sulfated tyrosine-containing proteins. Further, models to predict tyrosine O-sulfation sites in peptides or proteins are known. For example, the SwissProt Group at the Swiss Institute of Bioinformatics has developed an algorithm that predicts tyrosine-sulfated sites (see Sulfinator software program described in Monigatti et al., Bioinformatics 15:769-770 (2002)). Features recognized by tyrosylprotein sulfotransferases (TPST-1 and TPST-2) in a sulfation target site include acidic amino acids flanking a tyrosine. In general, tyrosine O-sulfation occurs on a tyrosine accessible in the trans-Golgi network, which is flanked within 5 residues on either side by at least 3 or 4 acidic amino acids.

Proteins comprising one or more sulfated tyrosines include adhesion molecules (CD44, endoglycan, glycoprotein Ibα, PSGL-1), coagulation factors (factor V, factor VIII, factor IX, factor X, fibrinogen γ chain, fibrogen β chain), matrix proteins (dermatopontin, fibromodulin, fibronectin, MAFp3, MAGP-1, nidogen, pherophorin I, procollagen type III, procollagen type V, vitronectin), serpins (α2-antiplasmin, heparin cofactor II), G-protein-coupled receptors (CCR5, CCR2B, CXCR4, CX3CR1, C5a receptor, TSH receptor), gastrin/CCK family members (gastrin, cholecystokinin, caerulein, cionin, sulfakinins), enzymes (aminopeptidase N, maltase-glucoamylase, PAM, sucrase-isomaltase), and various other proteins (such as α-conotoxin EpI, α-conotoxin PnIA/PnIB, α-fetoprotein, amyloid precursor protein, bone sialoprotein II, C4 α chain, chromogranin A, chromogranin B, choriogonadotropin α chain, FGF-7, hirudin, IgG2a-γchain, IgM-μ chain, M2B3 antigen, POMC, proenkephalin, prolactin, phyllokinin, phytosulfokine, secretogranin II, SGNE1, thyroglobulin, vitellogenin I, vitellogenin II, vitellogenin III).

Further, a variety of viral proteins are sulfated on tyrosine. Similarly, their cell receptor or binding partner proteins can comprise sulfated tyrosine. In particular, tyrosine sulfation may be significant in viral disease, such as disease associated with influenza A, rotavirus, and cytomegalovirus infection, as hemagglutin, V4, and US28 are predicted sulfotyrosine-containing proteins. Additionally, host cell recognition, viral attachment, and viral entry may be affected by tyrosine sulfation (for example on cellular or viral proteins), important to protein-protein interactions. Specifically, tyrosine sulfation of CCR5 may be important in HIV infection and/or disease progression.

VI. Kits

The invention also provides a kit for testing a sample for the presence of a sulfated tyrosine. The kit may also be used to test a sample for sulfated tyrosines present in proteins comprising sulfated tyrosine as listed above, for example.

The antibodies may further be provided in a diagnostic kit for use in performing one or more of the detection methods described above, to detect a peptide or protein comprising a sulfotyrosine. Such a kit may contain other components, packaging, instructions, or other material to aid the detection of the protein and use of the kit. The kit comprises the antibodies of the invention or active portions thereof. The antibody protein can be provided in an appropriate buffer or solvent, or alternatively the antibody protein can be lyophilized, for example. The antibody protein can also be directly or indirectly linked to an agent that aids in visualization, purification, or isolation of the antibody. For example, the antibody of the invention may be conjugated to a detectable label or an affinity tag. The kit optionally comprises a buffer, which can be an aqueous buffer, e.g., PBS. Further the kit optionally comprises a container, such as a reaction vessel for performing a detection assay. Such a kit may contain other components, packaging, instructions, such as a sulfotyrosine-containing control, a detection reagent, or other material to aid the detection of the protein and/or the use of the kit.

VII. Proteomics Methods

The antibodies disclosed herein are novel reagents for in vitro methods to identify and detect changes in the protein complement of a genome, or the proteome. The posttranslational modification of proteins can be associated with acute or chronic disease. The novel antibodies allow rapid identification of tyrosine sulfate modification, and improved proteomics methods to detect proteins comprising a sulfated tyrosine.

Accordingly, in another aspect the sulfated tyrosine specific antibodies are used in methods to detect proteins comprising a sulfated tyrosine residue, the method comprising separating a biological sample, and adding an antibody that specifically binds to sulfated tyrosine, thereby identifying proteins comprising a sulfated tyrosine residue.

In some embodiments, a biological sample is obtained from an animal, prepared, and fractionated. In some instances, the biological sample is prefractionated to prepare a set of subproteomes. Fractionation methods exploit specific protein characteristics, such as their inherent chemical properties, including biospecificity, hydrophobicity, or charge, or differential cellular location. Two-dimensional gel electrophoresis may be used to separate proteins. In certain cases, separation is carried out in the first dimension by isoelectric focusing, which separates proteins by their isoelectric point (pl). Proteins are resolved in a second dimension by, for example, their relative molecular mass in an SDS-PAGE analysis. Additional protein separation methods include ion exchange chromatography, size exclusion chromatography, reversed-phase high-performance liquid chromatography ((RP)-HPLC), capillary electrophoresis, capillary isoelectric focusing, and capillary zone electrophoresis, for example. One, two, three, or various multi-step fractionation methods are known in the art. Affinity chromatography is also used to separate or fractionate a biological sample. Separation may be carried out under native or denaturing conditions (see, e.g., Arrell et al., Circulation Res. 88:763-773 (2001)).

Protein identification follows protein separation in proteomics methods, and the methods provided herein detect sulfated tyrosine with a novel antibody that specifically binds to sulfated tyrosine, but not to unmodified or phosphorylated tyrosine, for example. One skilled in the art would appreciate that the methods to detect a protein comprising a sulfated tyrosine that are described above will adapt to proteomics methods.

VIII. Screening Methods

Yet another aspect of the invention provides a method of identifying therapeutic agents useful in the treatment of disorders associated with a sulfotyrosine containing protein. For example, an agent that modulates (increases or decreases) binding of a sulfated tyrosine specific antibody to its antigen may be identified as a therapeutic agent. Methods to screen for agents useful in treatment of a disorder associated with a protein comprising sulfotyrosine, such as the proteins listed above, are contemplated. Further, methods to screen for agents useful in treating viral or other infection are contemplated. Appropriate screening assays, e.g., ELISA-based assays, are known in the art. In such a screening assay, a first binding mixture is formed by combining an antibody of the invention and a ligand, e.g., a protein comprising a sulfated tyrosine; and the amount of binding between the ligand and the antibody in the first binding mixture (M0) is measured. A second binding mixture is also formed by combining the antibody, the ligand, and a compound or agent to be screened; and the amount of binding between the ligand and the antibody in the second binding mixture (M1) is measured. The amounts of binding in the first and second binding mixtures are then compared, for example, by calculating the M1/M0 ratio. The compound or agent is considered to be capable of inhibiting binding activity if a decrease in binding in the second binding mixture as compared to the first binding mixture is observed. The formulation and optimization of binding mixtures is within the level of skill in the art, such binding mixtures may also contain buffers and salts necessary to enhance or to optimize binding, and additional control assays may be included in the screening assay of the invention.

Compounds found to reduce the antibody-ligand binding by at least about 10% (i.e., M1/M0<0.9), preferably greater than about 20%, 30%, 40%, or 50% may thus be identified and then, if desired, secondarily screened for the capacity to inhibit the activity in other assays such as the binding to other ligands, and other cell-based and in vivo assays as described in the Examples.

IX. Method of Treating Sepsis and Systemic Inflammatory Response Syndrome

The antibodies of the present invention are useful to prevent or treat sepsis, septic shock, and systemic inflammatory response syndrome in animals, including mammals such as humans. Systemic inflammatory response syndrome (SIRS) includes an acute inflammatory reaction triggered by infection, pancreatitis, burn, or trauma, for example. Sepsis, in particular, may be caused by an infection (such as, e.g., a bacterial, viral, fungal, or parasitic infection) with systemic manifestations of inflammation. For example, sepsis may be caused by gram-positive or gram-negative bacteria such as Enterbacteriacae, Klebsiella species, Escherichia coli, Pseudomonas aeruginosa, Listeria monocytogenes, Neisseria meningitidis, Streptococcus pneumoniae, Staphylococcus aureus, Streptococcus pyogenes, Streptococcus pneumoniae, Haemophilus influenzae type b, Salmonella, and Group B streptococci. Sepsis may also be caused by, fungal e.g., Candida, infections. The infection may be an infection of the blood, it may be another systemic infection, or it may be localized, for example. Sepsis is characterized by a combination of increased coagulation (coagulopathy), decreased fibrinolytic activity, and a systemic inflammatory response. Healy, Ann. Pharmacother. 36:648-654 (2002). Mortality may be as high as 25-90%. Beers and Berkow, Eds., The Merck Manual, 17th ed., John Wiley & Sons (1999).

The term systemic inflammatory response syndrome (SIRS), as used herein, encompasses the terms sepsis, septic shock, severe sepsis, and septicemia. SIRS may be caused by, e.g., pancreatitis, burn, or trauma.

Sepsis and SIRS, for example, may be associated with hypoperfusion, hypotension, or acute organ dysfunction (such as, e.g., dysfunction of the kidneys, liver, gall bladder, bowel, skin, or lungs). Detection of infection, accompanied by one or more symptoms of a systemic inflammatory response may be used to identify sepsis, septic shock, or septicemia, for example. An individual having sepsis or SIRS may have confusion or delirium, chills, shaking, fever (a temperature greater than 38° C.), hypothermia (a temperature less than 36° C.), a rapid heart beat (heart rate greater than 90 beats/minute), hyperventilation (respiratory rate greater than 20 breaths/minute or P_{CO2} less than 32 mm Hg). Laboratory tests indicating a bacterial infection of the blood, a leukocyte count less of than 4,000 cells/mm³ or more than 12,000 cells/mm³, more than 10% immature neutrophils, acidosis, or a low platelet count (such as less than 50,000 platelets/μL) may also indicate sepsis.

Elevated levels, e.g., in blood, of endogenous mediators of inflammation are associated with these systemic inflammatory response syndromes. SIRS may be detected and/or quantified by elevated levels of such endogenous mediators of inflammation or other biomarkers associated with SIRS. For example, elevated levels of bacteria, endotoxin, TNF-α, leukocyte-produced oxidants, procalcitonin, leukocyte high-affinity Fc receptor (CD64), serum C-reactive protein, high mobility group protein 1, plasma D-dimer, IL-1 (e.g., IL-1β), IL-6, IL-8, or platelet activating factor (PAF) may be associated with sepsis or SIRS (see, e.g., Healy, Ann. Pharmacother. 36:648-654 (2002) and U.S. Patent Application Pub. Nos. 2005/0042202 A1, 2005/0181993 A1, 2004/019263 A1, 2004/0214756 A1, and references cited therein). For example, a level of TNF-α higher than 25 pg/ml, such as 30, 40, 50, 60, 70, 80, 90, 100, 110, 120, 130, 140, or 150 pg/ml, or a level of C-reactive protein greater than 0.1, 0.2, 0.3, 0.4, 0.5, 0.6, 0.7, 0.8, 0.9, or 1.0 mg/dl may be associated with sepsis or SIRS. Decreased levels of plasminogen, antithrombin III, protein C, thrombomodulin, and endothelial protein C receptor may also be associated with sepsis or SIRS (Healy, Ann. Pharmacother. 36:648-654 (2002)).

Detection of a reduction in one or more symptoms or clinical manifestations of SIRS and/or sepsis, for example, may be used to determine efficacy or disease progression. The antibodies of the present invention can be used to decrease the tendency of the blood to coagulate, for example, which may be useful in the treatment of sepsis. In certain embodiments, the tendency of the blood of an individual to coagulate is reduced at least 10%, such as, e.g., at least 15, 20, 30, 40, 50, 60, 62, 64, 66, 68, or 70% upon administration of one or more of the presently disclosed antibodies. In some embodiments, the decreased coagulation may be observed for at least 5, 10, 20, 30, 40, 50, or 60 minutes, and/or at least 1, 2, 3, 4, 5, 6, 7, 8, 10, 12, 14, 16, 18, 20, 22, or 24 hours. In other embodiments, the decreased coagulation may be observed for 1, 2, 3, 4, 5, 10, 15, or more days. Similarly, the effect may be complete by an indicated time point. Suitable assays for measuring blood coagulability will be apparent to one of skill in the art, and include routine clinical coagulation tests. Similarly, assays to measure levels of endogenous mediators of inflammation are well known, and include the prothrombin time/international normalized ratio (PT/INR) test, activated partial thromboplastin time (aPTT) test, thrombin time (TT) test, whole blood clotting time test, platelet number and function assays, factor activity assay, reptilase time test, template bleeding time test, activated coagulation time test, and the thromboelastograph (TEG tracing) test.

In certain embodiments, the immune response of an individual is reduced at least 10%, such as, e.g., at least 15, 20, 30, 40, 50, 60, 62, 64, 66, 68, or 70% upon administration of one or more of the presently disclosed antibodies, as measured by, for example, levels of TNF-α, leukocyte-produced oxidants, procalcitonin, leukocyte high-affinity Fc receptor (CD64), serum C-reactive protein, high mobility group protein 1, IL-1 (e.g., IL-1β), IL-6, IL-8, or platelet activating factor (PAF). In other embodiments, administration of one or more of the presently disclosed antibodies results in a decrease in bacterial or bacterial endotoxin levels.

The antibodies or antibody compositions of the present invention are administered in therapeutically effective amounts. Generally, a therapeutically effective amount may vary with the subject's age, condition, and sex, as well as the severity of the medical condition in the subject. The dosage may be determined by a physician and adjusted, as necessary, to suit observed effects of the treatment. Toxicity and therapeutic efficacy of such compounds can be determined by standard pharmaceutical procedures in vitro (i.e., cell cultures) or in vivo (i.e., experimental animal models), e.g., for determining the LD₅₀ (the dose lethal to 50% of the population) and the ED₅₀ (the dose therapeutically effective in 50% of the population). The dose ratio between toxic and therapeutic effects is the therapeutic index (or therapeutic ratio), and can be expressed as the ratio LD₅₀/ED₅₀. Antibodies that exhibit therapeutic indices of at least 0.5, 1, 1.5, 2, 3, 4, 5, 6, 7, 8, 9, 10, and 20 are described herein. For antibodies with a narrow therapeutic index, i.e., a ratio of less than 2, titration and patient monitoring may be indicated.

The data obtained from in vitro assays and animal studies, for example, can be used in formulating a range of dosage for use in humans. The dosage of such compounds lies preferably within a range of circulating concentrations that include the ED₅₀ with low, little, or no toxicity. The dosage may vary within this range depending upon the dosage form employed and the route of administration utilized. For any antibody used in the present invention, the therapeutically effective dose can be estimated initially from in vitro assays. A dose may be formulated in animal models to achieve a circulating plasma concentration range that includes the IC₅₀ (i.e., the concentration of the test antibody which achieves a half-maximal inhibition of symptoms) as determined in in vitro experiments. Levels in plasma are measured, for example, by high performance liquid chromatography. The effects of any particular dosage can be monitored by a suitable bioassay, such as a coagulation assay.

Generally, the compositions are administered so that antibodies or their binding fragments are given at a dose between 1 μg/kg and 30 mg/kg, 1 pg/kg and 10 mg/kg, 1 μg/kg and 1 mg/kg, 10 μg/kg and 1 mg/kg, 10 μg/kg and 100 μg/kg, 100 μg and 1 mg/kg, and 500 μg/kg and 1 mg/kg. In some embodiments, the antibodies are given as a bolus dose, such as a single bolus dose, to maximize the circulating levels of antibodies for the greatest length of time after the dose. Continuous infusion may also be used, optionally after a bolus dose.

The sulfotyrosine specific antibodies disclosed herein may be administered in combination with one or more anti-SIRS or anti-sepsis agents. For example, the sulfotyrosine specific antibodies may be administered in combination with antibiotics (e.g., beta-lactam, aminoglycoside, macrolide, tetracycline, peptide, polyene, sulfonamide, or nitrofuran antibiotics), as well as with antiviral (e.g., famvir or acyclovir), antifungal, or antiparasitic agents. For example, the sulfotyrosine specific antibodies may be administered with one or more of amikacin, amphotericin, ampicillin, augmentin, aztreonam, bacitracin, carbopenem, cefotaxime, ceftazidimine, ceftriaxone, cephalosporin, imipenem, penicillin, gentamicin, gramicidin, polymyxin, maxalactam, metronidazole, nalidixic acid, netilmicin, tobramycin, ureidopenicillin, and vancomycin.

The sulfotyrosine specific antibodies disclosed herein may be administered with anti-inflammatory agents (e.g., high dose corticosteroids, low dose corticosteroids, glucocorticoids (including hydrocortisone and fludrocortisone), pentoxifylline, immunoglobulins, or interferon gamma), as well as agents that increase blood pressure. The sulfotyrosine specific antibodies disclosed herein may be administered in combination with agents that target tumor necrosis factor (TNF), such as TNF-specific antibodies, anti-TNF antibody fragments (such as, e.g., afelimomab), or soluble TNF receptors; interleukin-1 (IL-1) receptor antagonists; phospholipase A2 inhibitors; ibuprofen or other cyclooxygenase inhibitors; thromboxane inhibitors such as dazoxiben and ketoconazole; PAF antagonists and PAF acetylhydrolase; agents that target free radicals such as N-acetylcysteine or selenium; agents that target nitric oxide such as N-methyl-1-arginine; and bradykinin antagonists. In another embodiment, the sulfotyrosine specific antibodies may be administered in combination with anti-coagulopathy agents such as antithrombin III, tissue factor pathway inhibitor (TFPI, such as, e.g., tifacogin), or activated protein C (e.g., drotrecogin alfa), or with anticoagulants such as heparin or warfarin. In one aspect, one or more sulfotyrosine specific antibodies of the invention are administered with insulin to regulate glycaemia. In another aspect, the sulfotyrosine specific antibodies are administered with a therapeutic agent that is a fusion protein with an antibody Fc fragment.

In some embodiments, the sulfotyrosine specific antibodies are administered with one or more of dopamine, norepinephrine, mannitol, furosemide, digitalis, pyridoxylated hemoglobin polyoxyethylene, prostaglandin E1, granulocyte colony stimulation factor (GCSF), and antibodies to various antigens on bacterial cell walls or to bacterial endotoxin.

The present invention provides compositions comprising the presently disclosed antibodies. Such compositions may be suitable for pharmaceutical use and administration to patients. The compositions typically comprise one or more antibodies of the present invention and a pharmaceutically acceptable excipient. As used herein, the phrase “pharmaceutically acceptable excipient” includes any and all solvents, dispersion media, coatings, antibacterial and antifungal agents, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. It may also be possible to obtain compositions which may be topically or orally administered, or which may be capable of transmission across mucous membranes. The administration may, for example, be intravenous, intraperitoneal, intramuscular, intracavity, subcutaneous, or transdermal.

Solutions or suspensions used for intradermal or subcutaneous application typically include one or more of the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes, or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Oral compositions generally include an inert diluent or an edible carrier. They can be enclosed in gelatin capsules or compressed into tablets. For the purpose of oral therapeutic administration, the antibodies can be incorporated with excipients and used in the form of tablets, or capsules. Pharmaceutically compatible binding agents, and/or adjuvant materials can be included as part of the composition. The tablets, pills, capsules, and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, antibodies are delivered in the form of an aerosol spray from pressured container or dispenser, which contains a suitable propellant, e.g., a gas such as carbon dioxide, or a nebulizer.

Systemic administration can also be by transmucosal or transdermal means. For example, in case of antibodies that comprise the Fc portion, compositions may be capable of transmission across mucous membranes (e.g., intestine, mouth, or lungs) via the FcRn receptor-mediated pathway (U.S. Pat. No. 6,030,613). Transmucosal administration can be accomplished, for example, through the use of lozenges, nasal sprays, inhalers, or suppositories. For transdermal administration, the active compounds are formulated into ointments, salves, gels, or creams as generally known in the art. For transmucosal or transdermal administration, penetrants appropriate to the barrier to be permeated are used in the formulation. Such penetrants are generally known in the art, and include, for example, detergents, bile salts, and fusidic acid derivatives.

In some instances, oral or parenteral compositions are formulated in dosage unit form for ease of administration and uniformity of dosage. Dosage unit form as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of formulating such an active compound for the treatment of individuals.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### EXAMPLES

### Example 1

Isolation of the antibodies of the invention. Single chain Fv fragments (scFv's) were isolated from human phage display libraries using the fully sulfated and glycosylated human PSGL-1 19.ek.Fc fusion protein (SEQ ID NO:30). A scFv phagemid library, which is an expanded version of the 1.38×10¹⁰ library (Vaughan et al., Nature Biotech. 14:309-314 (1996)), was used to select antibodies that bind to human and rat PSGL-1.

Panning selections were performed as follows. The PSGL-1 19.ek.Fc fusion protein (10 μg/ml in 10 mM NaHCO₃, pH 9.6) or control IgG (50 μg/ml) was coated onto a 96-well plate at 100 μL/well and incubated overnight at 4° C. Wells were washed in PBS and blocked for 1 hour at 37° C. in 3% MPBS (3% ‘Marvel’ skimmed milk powder in PBS). Purified phage (10¹² transducing units) in 100 μL of 3% MPBS also containing 400 μg/ml of the control IgG were added to blocked control IgG wells and incubated at room temperature for 1 hour. The blocked phage were then transferred to the blocked PSGL-1 19.ek.Fc protein coated wells and incubated for 1 hour at room temperature. The wells were first washed 10 times with PBST (PBS containing 0.1% v/v Tween 20), then washed 10 times with PBS. Bound phage particles were eluted with 100 μL of 100 mM triethylamine for 10 minutes at room temperature, then neutralized with 50 μL 1 M Tris HCl, pH 7.4.

The eluted phage particles were used to infect 10 ml of exponentially growing E. coli TG1. The infected cells were grown in 2TY broth for 30 minutes at 37° C. stationary, followed by 30 minutes at 37° C. with aeration. The cells were then streaked onto 2TYAG plates (2TY medium containing 100 μg/ml ampicillin and 2% glucose). The plates were incubated overnight at 30° C. Output colonies were scraped off the plates into 10 ml 2TY broth and 15% glycerol was added for storage at −70° C.

Glycerol stock cultures from the first-round panning selection were superinfected with helper phage and rescued to give scFv antibody-expressing phage particles for the second round of panning. Two rounds of panning were carried out in this way.

Soluble selection on PSGL-1 19.ek.Fc was done using biotinylated PSGL-1 19.ek.Fc protein at a concentration of 100 nM. A scFv library, described above, was used. Purified scFv phage (10¹² transducing units) in 1 ml 3% MPBS were blocked for 30 minutes, then biotinylated PSGL-1 19.ek.Fc protein was added, and the sample was incubated at room temperature for 1 hour. Phage/antigen was added to 250 μL of Dynal M280 strepavidin magnetic beads (Dynal, Lake Success, N.Y.) that had been blocked for 1 hour at 37° C. in 1 ml of 3% MPBS, and the sample was incubated an additional 15 minutes at room temperature. The beads were captured using a magnetic rack and washed four times in 1 ml of 3% MPBS/0.1% (v/v) Tween 20, followed by three washes in PBS. After the last PBS wash, the beads were resuspended in 100 μL PBS and used to infect 5 ml of exponentially growing E. coli TG1 cells. Cells and phage were incubated for 1 hour at 37° C. (30 minutes stationary, 30 minutes shaking at 250 rpm), then spread on 2TYAG plates. Plates were incubated at 30° C. overnight and colonies visualized the next day. Output colonies were scraped off the plates and phage rescued as described above.

A second round of soluble selection was then carried out. Output colonies from selections were picked into duplicate 96 well plates containing 1 ml of 2TYAG. Samples were tested either as polyethylene glycol (PEG) precipitated phage supernatants or as crude bacterial periplasmic extracts. Periplasmic scFv production was induced by addition of 1 mM IPTG to exponentially growing cultures and incubation overnight at 30° C. Crude scFv-containing periplasmic extracts were obtained by subjecting the bacterial pellets from the overnight growth to osmotic shock. The pellets were re-suspended in 20% (w/v) sucrose, 1 mM Tris-HCl, pH 7.5 and cooled on ice for 30 minutes. Following centrifugation, the extracts were diluted to 5% in assay buffer (10 mM MOPS, 150 mM NaCl, 1 mM CaCl2, 1 mM MgCl2, pH 7.5) and used in the assays.

Phage production was induced by superinfection with helper phage followed by overnight rescue at 30° C. Overnight phage preparations were PEG precipitated before use in the assays. The phage-containing culture supernatants were transferred to a fresh plate and ⅕th volume of 20% (w/v) PEG-8000, 250 mM NaCl was added followed by cooling on ice for 30 minutes. Following centrifugation, the protein pellets were re-suspended in 150 μL assay buffer and were used in the assay at 5%.

ScFv clones that demonstrated the ability to neutralize the binding of biotinylated PSGL-1 19.ek.Fc protein to soluble P-selectin immobilized on plastic in a 96 well plate (ELISA format), were grown in 2TYAG. Periplasmic scFv production was induced by addition of 1 mM isopropylthiogalactoside (IPTG) to exponentially growing cultures at OD600=0.9-1.1 and incubated for 3.5 hr at 30° C. Crude scFv-containing periplasmic extracts were obtained by subjecting the bacterial pellets from the 500 mL cultures to osmotic shock. Pellets were resuspended in 20 ml 1 M NaCl, 1 mM EDTA in PBS and cooled on ice for 30 minutes. Following centrifugation, the supernatants containing the scFv were mixed with NiNTA (Qiagen, Valencia, Calif.) and allowed to bind at 4° C. overnight. The NiNTA slurry was loaded onto a polyprep column (Biorad, Cambridge, Mass.), washed, and eluted with PBS containing 250 mM imidazole. The scFv's were concentrated and buffer exchanged to PBS using a Centricon-10 (Millipore, Billerica, Mass.). The scFv protein concentrations were determined using a micro BCA protein assay (Pierce, Rockford, Ill.).

The two scFvs described herein were sequenced using standard DNA sequencing techniques. The nucleic acid and amino acid sequences for PSG1 and PSG2 scFv's appear in FIG. 1 and FIG. 2, respectively. Variable domain sequences are indicated in bold.

### Example 2

Generation of full-length antibodies. The scFv's were then converted to full length bivalent antibodies (Thompson, J. Immunol. Methods 227:17-29 (1999)). In this context, full-length antibody refers to the single chain antibody reformatted to IgG. The variable heavy and light chains of the selected clones were amplified by PCR from scFv's of Example 1. The PCR primers contained cloning sites which facilitated insertion into the expression vectors. The vector pED6_HC_gamma4 (containing a heavy chain leader sequence and the CH1-CH3 domains of human IgG4) and the vector pED6_LC (containing a light chain leader sequence and the C domain of human lambda) were transiently expressed in COS cells by TransIt®-based transfection (Mirus Corporation, Madison, Wis.). These vectors are described in Kaufman et al., Nucleic Acids Res. 19:4485-4490 (1991).

For the generation of stable CHO cells, the coding region fragments for the variable heavy and light chains were ligated into separate mammalian expression vectors. CHO 153.8 PA DUKX cells were cotransfected with a lipofectine-based method (Gibco-BRL, Gaithersburg, Md.) after both heavy and light chain plasmids were linearized. Clones were selected and maintained in alpha medium with 10% heat-inactivated, dialyzed fetal calf serum, 2 mM glutamine, 100 U/mL penicillin/streptomycin, and methotrexate ranging from 5 mM to 100 mM.

Clonal CHO lines exhibiting the desired productivity and growth phenotype were selected. The antibody production process was done using chemically defined medium free of animal-derived or human-derived components. The antibodies were purified by Protein A sepharose chromatography (Pharmacia, Uppsala, Sweden), concentrated, and buffer exchanged to PBS pH 7.2 using a Centricon® MW 30 (Millipore, Billerica, Mass.).

### Example 3

Competitive Binding Assays with PSG1 and PSG2. ScFv's and full-length antibodies were screened for the ability to inhibit the binding of biotinylated human PSGL-1 19.ek.Fc fusion protein or biotinylated rPSGL Ig (which contains the N-terminal 47 amino acids of human PSGL-1 fused to human Fc) to P-selectin or L-selectin in competitive enzyme-linked immunosorbent assay (ELISA) format.

Streptavidin-horseradish peroxidase 4 μg/mL (Southern Biotechnology Associates, Birmingham, Ala.) was incubated for 30 minutes at RT with 80 ng/mL biotinylated 19.ek.Fc fusion protein or biotinylated rPSGL-Ig to form a SA-HRP/biotinylated complex (for final concentration of 2 μg/mL SA-HRP, 40 ng/mL biotinylated fusion protein), the complex was then incubated for another 15 minutes at RT in the presence or absence of purified scFv or full length antibodies at different concentrations (for final concentration of 1 μg/mL SA-HRP, 20 ng/mL biotinylated fusion protein).

For these studies, flat microtiter plates (Maxi-Sorp, Nunc, Napeville, Ill.) or Costar (Corning, N.Y.) were coated with human P-selectin-Fc or human L-selectin-Fc at 1 μg/mL, 100 μL per well at 4° C. overnight in coating buffer (10 mM MOPS, 150 nM NaCl, 1 mM CaCl₂, 1 mM MgCl₂, pH 7.5). The next day, plates were washed with coating buffer, 0.05% Tween 20, 50 μg/mL BSA and blocked with 200 μL per well for one hour at RT with coating buffer, 0.1% gelatin (Bio-Rad, Cambridge, Mass.). The washed selectin coated plates were incubated for 30 minutes at RT with 100 μL SA-HRP-biotinylated complex with 3 μg/ml scFv's or 1.5 μg/ml mAbs 2× serial diluted. After washing 3 times the wells were incubated 10 minutes with 100 μL TMB (BioFX, Owings Mills, Md.). The reaction was stopped by adding 100 μL 0.18 M H₂S0₄, and the absorbance was read at 450 nm using a plate reader (Lab Systems, Helsinki, Finland).

The scFv's showed dose-dependent inhibition of biotinylated PSGL-19.ek.Fc binding to human P-selectin, human L-selectin, and rPSGL-Ig. Thus, the anti-sulfotyrosine scFv's PSG1 and PSG2 competitively inhibited the binding of PSGL-1 to its substrates P-selectin and L-selectin. The binding was specific as shown by lack of an irrelevant antibody 3D1 binding and dose-dependent of inhibition of positive control antibody KPL1.

The scFvs were converted to intact full-length bivalent antibodies as described in Example 2 (see also, Thompson, J. Immunol. Methods 227:17-29 (1999)). After full-length antibody conversion, the antibodies were tested by competitive ELISA using biotinylated human PSGL-1 19.ek.Fc fusion protein and biotinylated rPSGL Ig (data not shown). The specificity of binding was demonstrated by lack of inhibition with the irrelevant 3D1 antibody and a dose-dependent inhibition of positive control antibody KPL1. The bivalent antibodies demonstrated greater blocking activity relative to their corresponding monovalent scFv forms. Furthermore, the monoclonal antibodies inhibited binding of PSGL-19.ek.Fc to both P-selectin and L-selectin with IC 50's between 0.2 and 0.8 nM.

For cross reactivity, rat P-selectin Fc was coated on microtiter plates at 1 μg/ml. Biotinlylated rat-PSGL-1 at 50 ng/ml was competed with monoclonal antibodies started at 7.5 μg/ml 3× serial diluted as described for the human P or L selectin above. The binding was specific as shown by lack of an irrelevant antibody binding and dose-dependent inhibition of positive control antibody PSG2 (data not shown). In this assay, the human PSG2 antibody blocked binding of both human PSGL-19.ek.Fc to human P selectin and of rat-PSGL-1 binding to rat P selectin, while another human monoclonal PSG3 antibody that specifically binds to PSGL-19.ed.Fc blocks binding of PSGL-19.ek.Fc to human P-selectin only. The rat PSG G1 antibody blocked binding of rat-PSGL-1 to rat P-selectin, and the anti-murine PSGL-1 antibody, 4 RA10, does not block either. These results showed that unlike the other antibodies tested, PSG2 bound in a species-independent manner.

### Example 4

Peptides for characterization of antibody binding. To elucidate which determinant(s) within the PSGL-1 19.ek.Fc fusion protein were recognized by the human monoclonal antibodies, surface plasmon resonance was performed using a set of highly purified PSGL-1 19.ek peptides with varying degrees of sulfation and/or glycosylation (Somers et al., Cell 103:467-479 (2000)).

The generation of PSGL-1 19.ek peptides has been previously described (Somers et al., Cell 103:467-479 (2000)). Briefly, conditioned media from CHO cells transfected with PSGL-1 19.ek.Fc, Fucosyl transferase VII (FTVII), and CORE-2 cDNAs were purified with. Protein A. The purified PSGL-1 19.ek.Fc polypeptide was cleaved by enterokinase treatment. The cleaved protein was separated by Protein A sepharose and the resultant PSGL-1 19.ek peptide pool was resolved by anion exchange HPLC on a SuperQ anion exchange column. (TosoHaas, Montgomeryville, Pa.).

The major PSGL-1 19.ek peptide was the sulfoglycopeptide termed SGP-3, which is posttranslationally modified by sulfate on all three tyrosine residues (i.e., the residues corresponding to Tyr46, Tyr48, and Tyr51 of mature human PSGL-1), having the amino acid sequence of SEQ ID NO:30, and modified by SLex-capped O-glycan also found in PSGL-1 isolated from HL-60 cells (Wilkins et al., J. Biol. Chem. 271:18732-42 (1996)). SGP-1 and SGP-2 are forms of hyposulfated forms containing only one and two tyrosine sulfates, respectively (see SEQ ID NOs:39-44). Glycopeptide-1 (GP-1) contains no tyrosine sulfates (see SEQ ID NO:38). Sulfopeptide-1 (SP-1) contains no carbohydrate. These peptides and a synthetic peptide (AnaSpec, San Jose, Calif.) corresponding to the polypeptide portion of SGP-3 (SEQ ID NO:30) but lacking sulfated tyrosine were biotinylated at Lys residues as described previously (Somers et al., Cell 103:467-479, 2000). These biotinylated peptides were used to characterize the binding of the PSG1 and PSG2 antibodies using surface plasmon resonance.

GP-1 glycopeptide contains one O-linked glycan, lacks sulfated tyrosine, and has the amino acid sequence QATEYEYLDYDFLPETEPPRPMMDDDDK (SEQ ID NO:38). SGP-1 is the monosulfated glycopeptide 19.ek, and is a mixture of peptides having the amino acid sequences QATEyEYLDYDFLPETEPPRPMMDDDDK (SEQ ID NO:39), QATEYEyLDYDFLPETEPPRPMMDDDDK (SEQ ID NO:40), and QATEYEYLDyDFLPETEPPRPMMDDDDK (SEQ ID NO:41). SGP-2 is the disulfated glycopeptide 19.ek, and is a mixture of peptides having the amino acid sequences QATEYEyLDyDFLPETEPPRPMMDDDDK (SEQ ID NO:42), QATEyEYLDyDFLPETEPPRPMMDDDDK (SEQ ID NO:43) and QATEyEyLDYDFLPETEPPRPMMDDDDK (SEQ ID NO:44). SGP-3 is the trisulfated glycopeptide 19.ek, and has the amino acid sequence QATEyEyLDyDFLPETEPPRPMMDDDDK (SEQ ID NO:30).

Surface plasmon resonance binding analysis. A BIAcore 2000 instrument (BIAcore AB, Uppsala, Sweden) was used to analyze the interactions between the identified antibodies and biotinylated PSGL-1 19.ek.Fc or derived peptides. Binding experiments were performed at 25° C. using streptavidin-coated sensor chips (BIAcore) and HBS-P buffer (20 mM HEPES [pH 7.4], 150 mM NaCl and 0.005% polysorbate 20 v/v) adjusted to 1 mM for both CaCl₂ and MgCl₂. The streptavidin on the sensor surfaces were conditioned with three one-minute injections of a solution containing 1 M NaCl and 25 mM NaOH. The chips were regenerated with 5 μL of 0.1% TFA and equilibrated with running buffer. Curves were corrected for non-specific binding by an online baseline subtraction of ligand binding to streptavidin surface in control flow channel. Binding kinetics were analyzed using BIAevaluation software (V2.1; Pharmacia Biosensor, Uppsala, Sweden). The response representing the mass of bound monoclonal antibodies was measured in resonance units (RU). Flow cell one (FC1) was used as reference surface. The human monoclonal antibodies were diluted in HBS-P buffer at 200 nM and 100 nM based on OD₂₈₀. The diluted antibodies were injected at flow rates of 2, 10, 30, 50, and 100 μL/min to determine the active concentration. Binding kinetics of human anti-PSGL-1 monoclonal antibodies to the immobilized PSGL-1 19.ek.Fc was determined under partial mass transport limitations by triplicate injections at a concentration range (0-100 nM) onto the immobilized biotinlylated PSGL-1 19.ek.Fc peptide at a flow rate of 30 μL/min, following injection for two minutes. Dissociation was monitored for ten minutes at the same flow rate. Kinetic data for the interaction between monoclonal antibodies and biotinlylated PSGL-1 19.ek.Fc fusion protein found a binding affinity for PSG1 of approximately 7.5×10⁹ M⁻¹, and for PSG2 of approximately 3.2×10¹⁰ M⁻¹.

Peptide binding. Antibodies (PSG-1, PSG-2, KPL-1, PSL-275, and 3D1, for example) were passed over a streptavidin chip coated with synthetic peptides.

Flow cell 1 (FC1) was left as a blank surface for double reference. The streptavidin chip was coated on flow cell 2 (FC2) with an unglycosylated and unsulfated synthetic peptide 19.ek, that corresponds to the polypeptide portion of SGP-3, and has the amino acid sequence OATEYEYLDYDFLPETEPP (SEQ ID NO:37). The glycopeptide GP-1, or 19.ek having minimal sulfation was coated on flow cell 3 (FC3). Sulfated and glycosylated peptide SGP-3 was coated on flow cell 4 (FC4).

Human monoclonal antibodies PSG1 and PSG2 as well as PSL-275, KPL1 and, an irrelevant human monoclonal 3D1 were injected in duplicate at 100 nM through all flow cells.

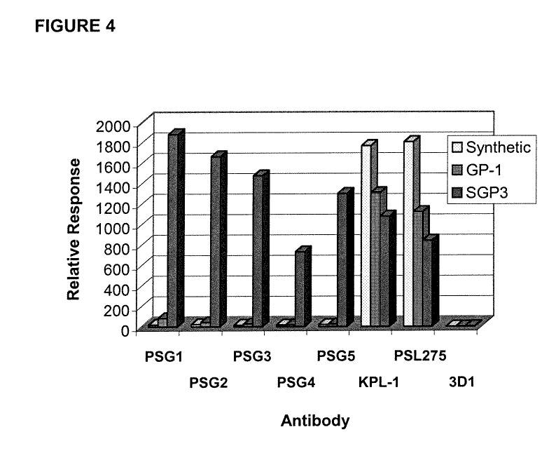

The results are shown in FIG. 4. PSL 275 (which is a murine monoclonal anti-human PSGL-1 antibody raised against a human PSGL-1 synthetic peptide) and KPL1 both bound to the synthetic peptide lacking sulfated tyrosine. In contrast, the human monoclonals PSG1 and PSG2 did not bind to the synthetic peptide. In addition, the PSG1 and PSG2 binding to the glycopeptide, GP-1, was very minimal, i.e., did not show specific binding. The PSG1 and PSG2 human monoclonal antibodies required the sulfo-glycopeptide SP-1 in order to bind. These data show that these human monoclonal antibodies recognized an epitope comprising at least one sulfated tyrosine.

### Example 5

PSG1 and PSG2 are specific for tyrosine sulfate in multiple proteins. To determine the specificity of the human PSG1 and PSG2 antibodies, we selected two additional proteins containing sulfated tyrosine residues, murine PSGL-1.Fc and GPIbα.Fc. The amino acids that are adjacent to or near the sulfated tyrosines in murine PSGL-1 differ from the amino acid context surrounding human PSGL-1 sulfated tyrosines. Simlarly, the context for the sulfotyrosine in GPIbα is distinct.

Murine PSGL-1.Fc is comprised of the mature murine PSGL-1 amino terminal 45 amino acids, with the sequence, QVVGDDDFEDPDyTyNTDPPELLKNVTNTVAAHPELPTTVVMLER (SEQ ID NO:45) fused to a human IgG1 Fc (see U.S. Pat. No. 6,277,975 B1 at e.g., col. 44, line 61 to col. 45, line 5 and sequences in the listing identified as SEC ID NOs:35 and 36 for human PSGL-1.Fc fusion sequences). The human GPIbα protein used in this experiment is a platelet glycoprotein containing a cluster of three sulfated tyrosines with the peptide sequence DLYDYYPEED (SEQ ID NO:27), or DLyDyyPEED (SEQ ID NO:31), (see U.S. Patent Application Pub. No. US 2003/0091576 A1). The GPIbα DNA sequence is at SEQ ID NO:46 (see, for example, U.S. 2003/0091576 A1 for other GPIbα sequences or fragments that comprise the sulfotyrosine-containing region.

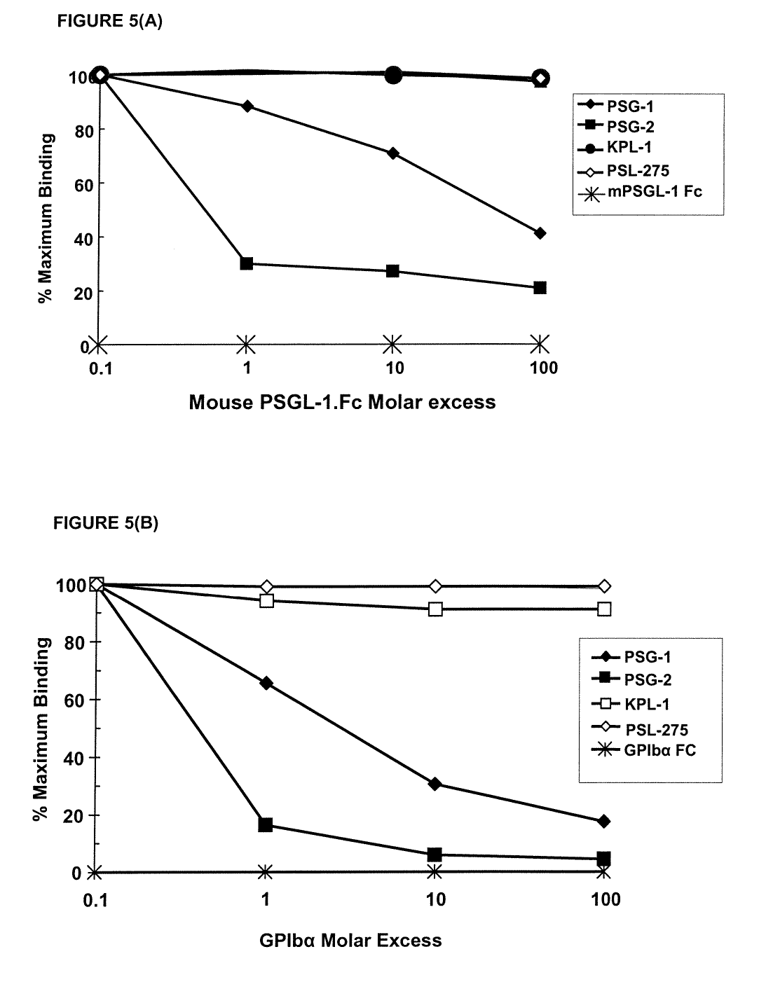

The binding of human monoclonal antibodies (25 nM) to the immobilized PSGL-1 19.ek.Fc (comprising SEQ ID NO:37 and a human IgG1 Fc as described in Somers et al., Cell 103:467-479 (2000)) was competed with 100, 10, 1, and 0 molar excess of murine PSGL-1.Fc (FIG. 5(A)) or GPIbα-Fc (FIG. 5(B)). Other monoclonal antibodies that specifically bind to the 19 amino acid human PSGL-1 peptide (PSGL-1 19.ek.Fc) are not competitively inhibited by the two unrelated sulfated tyrosine-containing proteins. These were either a mouse PSGL-1 Fc fusion protein or a human GPIbα.Fc fusion protein (data not shown). In contrast, both the murine PSGL-1.Fc or GPIbα.Fc did inhibit PSG1 and PSG2 binding in a dose dependent manner. These results suggest that PSG1 and PSG2 bind to other peptides containing sulfated tyrosine in addition to the sulfated peptide used in panning and selection from the phagemid library.

### Example 6

Epitope mapping of PSG2. Fmoc-protected amino acids and cellulose membranes modified with polyethylene glycol were purchased from Intavis. Fmoc-protected β-alanine was purchased from Chem-Impex (Wood Dale, Ill.). The arrays were defined on the membranes by coupling a β-alanine spacer, followed by elongation of the peptide chain. Peptides were synthesized using standard DIC/HOBt coupling chemistry as described previously. See, e.g., Molina et al., Pept. Res. 9:151-155 (1996) and Frank et al., Tetrahedron 48:9217-9232 (1992). Activated amino acids were spotted using an Abimed ASP 222 robot. Washing and deprotection steps were done manually and the peptides were N-terminally acetylated after the final synthesis cycle.

Following peptide synthesis and side chain deprotection, the membranes were washed in methanol for 10 minutes and in blocker (1% casein in TBD) for 10 minutes. The membranes were then incubated with 1 μg/mL of PSG2 in TBS for 1 hour with gentle shaking. The membranes were washed 4 times for 2 minutes in TBS and then probed with an HRP-conjugated anti-Fc antibody in blocker. After washing with TBS, bound protein was visualized using SuperSignal West reagent (Pierce) and a digital camera (Alphalnnotech FluorImager). Signal intensity reflects the amount of protein bound at each spot.

The binding epitope for PSG2 was mapped using the peptides listed in Table 4.

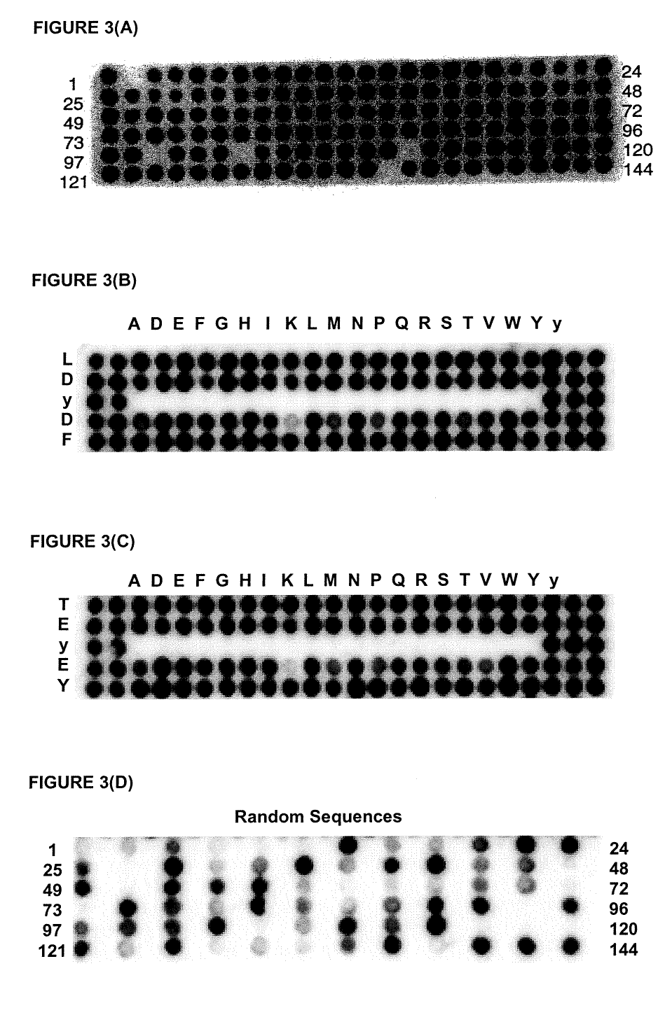

TABLE 4

| | FIG. 3(A) | | FIG. 3(D) | |
|---|---|---|---|---|
| Peptide | | SEQ ID | | SEQ ID |
| No. | Peptide Sequence | NO. | Peptide Sequence | NO. |
| 1 | QATEyEyLDyDFL | 47 | AAyAA | 191 |
| 2 | QATEYEYLDYDFL | 48 | AAYAA | 192 |
| 3 | QATEyEYLDYDFL | 49 | AyA | 193 |
| 4 | QATEYEyLDYDFL | 50 | AYA | 194 |
| 5 | QATEYEYLDyDFL | 51 | yA | 195 |
| 6 | QATEyEyLDYDFL | 52 | YA | 196 |
| 7 | QATEyEYLDyDFL | 53 | Ay | 197 |
| 8 | QATEYEyLDyDFL | 54 | AY | 198 |
| 9 | QATEyEyLDyDF | 55 | y | 199 |
| 10 | QATEyEyLDyD | 56 | Y | 200 |
| 11 | ATEyEyLDyDFL | 57 | y | 201 |
| 12 | ATEyEyLDyDF | 58 | Y | 202 |
| 13 | ATEyEyLDyD | 59 | FDyWN | 203 |
| 14 | TEyEyLDyDFL | 60 | FDYWN | 204 |
| 15 | TEyEyLDyDF | 61 | MMyQW | 205 |
| 16 | TEyEyLDyD | 62 | MMYQW | 206 |
| 17 | EyEyLDyDFL | 63 | YMyLN | 207 |
| 18 | EyEyLDyDF | 64 | YMYLN | 208 |
| 19 | EyEyLDyD | 65 | LEyFK | 209 |
| 20 | QATEyEYLDYDF | 66 | LEYFK | 210 |
| 21 | QATEyEYLDYD | 67 | LIyDY | 211 |
| 22 | ATEyEYLDYDFL | 68 | LIYDY | 212 |
| 23 | ATEyEYLDYDF | 69 | KPyYE | 213 |
| 24 | QATEyEyLDyDFL | 70 | KPYYE | 214 |
| 25 | QATEyEyLDyDFL | 71 | QWyFR | 215 |
| 26 | ATEyEYLDYD | 72 | QWYFR | 216 |
| 27 | TEyEYLDYDFL | 73 | GKyAK | 217 |
| 28 | TEyEYLDYDF | 74 | GKYAK | 218 |
| 29 | TEyEYLDYD | 75 | NVyET | 219 |
| 30 | EyEYLDYDFL | 76 | NVYET | 220 |
| 31 | EyEYLDYDF | 77 | RFyRN | 221 |
| 32 | EyEYLDYD | 78 | RFYRN | 222 |
| 33 | QATEYEyLDYDF | 79 | FFyTN | 223 |
| 34 | QATEYEyLDYD | 80 | FFYTN | 224 |
| 35 | ATEYEyLDYDFL | 81 | EIyLD | 225 |
| 36 | ATEYEyLDYDF | 82 | EIYLD | 226 |
| 37 | ATEYEyLDYD | 83 | MYyAF | 227 |
| 38 | TEYEyLDYDFL | 84 | MYYAF | 228 |
| 39 | TEYEyLDYDF | 85 | NDySA | 229 |
| 40 | TEYEyLDYD | 86 | NDYSA | 230 |
| 41 | EYEyLDYDFL | 87 | DDyFF | 231 |
| 42 | EYEyLDYDF | 88 | DDYFF | 232 |
| 43 | EYEyLDYD | 89 | ASyRH | 233 |
| 44 | QATEYEYLDyDF | 90 | ASYRH | 234 |
| 45 | QATEYEYLDyD | 91 | VRyFQ | 235 |
| 46 | ATEYEYLDyDFL | 92 | VRYFQ | 236 |
| 47 | ATEYEYLDyDF | 93 | QIyKV | 237 |
| 48 | QATEyEyLDyDFL | 94 | QIYKV | 238 |
| 49 | QATEyEyLDyDFL | 95 | PPyQD | 239 |
| 50 | ATEYEYLDyD | 96 | PPYQD | 240 |
| 51 | TEYEYLDyDFL | 97 | IFyLI | 241 |
| 52 | TEYEYLDyDF | 98 | IFYLI | 242 |
| 53 | TEYEYLDyD | 99 | KYyEL | 243 |
| 54 | EYEYLDyDFL | 100 | KYYEL | 244 |
| 55 | EYEYLDyDF | 101 | FIyNY | 245 |
| 56 | EYEYLDyD | 102 | FIYNY | 246 |
| 57 | QATEyEYLDyDF | 103 | TFyDK | 247 |
| 58 | QATEyEYLDyD | 104 | TFYDK | 248 |
| 59 | ATEyEYLDyDFL | 105 | QKySW | 249 |
| 60 | ATEyEYLDyDF | 106 | QKYSW | 250 |
| 61 | ATEyEYLDyD | 107 | QTyVA | 251 |
| 62 | TEyEYLDyDFL | 108 | QTYVA | 252 |
| 63 | TEyEYLDyDF | 109 | KVyTT | 253 |
| 64 | TEyEYLDyD | 110 | KVYTT | 254 |
| 65 | EyEYLDyDFL | 111 | GQyNM | 255 |
| 66 | EyEYLDyDF | 112 | GQYNM | 256 |
| 67 | EyEYLDyD | 113 | WHyLV | 257 |
| 68 | QATEYEyLDyDF | 114 | WHYLV | 258 |
| 69 | QATEYEyLDyD | 115 | WFyMA | 259 |
| 70 | ATEYEyLDyDFL | 116 | WFYMA | 260 |
| 71 | ATEYEyLDyDF | 117 | GWyKL | 261 |
| 72 | QATEyEyLDyDFL | 118 | GWYKL | 262 |
| 73 | QATEyEyLDyDFL | 119 | QVyKW | 263 |
| 74 | ATEYEyLDyD | 120 | QVYKW | 264 |
| 75 | TEYEyLDyDFL | 121 | VWyEM | 265 |
| 76 | TEYEyLDyDF | 122 | VWYEM | 266 |
| 77 | TEYEyLDyD | 123 | NHySM | 267 |
| 78 | EYEyLDyDFL | 124 | NHYSM | 268 |
| 79 | EYEyLDyDF | 125 | SSyQG | 269 |
| 80 | EYEyLDyD | 126 | SSYQG | 270 |
| 81 | QATEyEyLDYDF | 127 | KDyEP | 271 |
| 82 | QATEyEyLDYD | 128 | KDYEP | 272 |
| 83 | ATEyEyLDYDFL | 129 | HFyWF | 273 |
| 84 | ATEyEyLDYDF | 130 | HFYWF | 274 |
| 85 | ATEyEyLDYD | 131 | ISyVT | 275 |
| 86 | TEyEyLDYDFL | 132 | ISYVT | 276 |
| 87 | TEyEyLDYDF | 133 | YRyGL | 277 |
| 88 | TEyEyLDYD | 134 | YRYGL | 278 |
| 89 | EyEyLDYDFL | 135 | SHyWA | 279 |
| 90 | EyEyLDYDF | 136 | SHYWA | 280 |
| 91 | EyEyLDYD | 137 | KQyEY | 281 |
| 92 | QATEyEyLDyDFL | 138 | KQYEY | 282 |
| 93 | QATEyEyLDyDFL | 139 | PFyKS | 283 |
| 94 | QATEyEyLDyDFL | 140 | PFYKS | 284 |
| 95 | QATEyEyLDyDFL | 141 | PAyHN | 285 |
| 96 | QATEyEyLDyDFL | 142 | PAYHN | 286 |
| 97 | QATEyEyLDyDFL | 143 | HSyLN | 287 |
| 98 | DDFEDPDyTyNTD | 144 | HSYLN | 288 |
| 99 | DDFEDPDYTYNTD | 145 | GRyMW | 289 |
| 100 | DDFEDPDyTYNTD | 146 | GRYMW | 290 |
| 101 | DDFEDPDYTyNTD | 147 | KIyFT | 291 |
| 102 | DFEyPDySVyGTD | 148 | KIYFT | 292 |
| 103 | DFEYPDYSVYGTD | 149 | NNyFE | 293 |
| 104 | DFEyPDYSVYGTD | 150 | NNYFE | 294 |
| 105 | DFEYPDySVYGTD | 151 | SMyPG | 295 |
| 106 | DFEYPDYSVyGTD | 152 | SMYPG | 296 |
| 107 | DFEyPDySVYGTD | 153 | MKyGF | 297 |
| 108 | DFEyPDYSVyGTD | 154 | MKYGF | 298 |
| 109 | DFEYPDySVyGTD | 155 | HDyTA | 299 |
| 110 | GDTDLyDyyPEED | 156 | HDYTA | 300 |
| 111 | GDTDLYDYYPEED | 157 | QHyIY | 301 |
| 112 | GDTDLyDYYPEED | 158 | QHYIY | 302 |
| 113 | GDTDLYDyYPEED | 159 | QFyEW | 303 |
| 114 | GDTDLYDYyPEED | 160 | QFYEW | 304 |
| 115 | GDTDLyDyYPEED | 161 | AVyPP | 305 |
| 116 | GDTDLyDYyPEED | 162 | AVYPP | 306 |
| 117 | GDTDLYDyyPEED | 163 | YRyKW | 307 |
| 118 | QATEyEyLDyDFL | 164 | YRYKW | 308 |
| 119 | QATEyEyLDyDFL | 165 | IQyQK | 309 |
| 120 | QATEyEyLDyDFL | 166 | IQYQK | 310 |
| 121 | QATEyEyLDyDFL | 167 | PIyWD | 311 |
| 122 | AATEyEyLDyDFL | 168 | PIYWD | 312 |
| 123 | QAAEyEyLDyDFL | 169 | KAyGL | 313 |
| 124 | QATAyEyLDyDFL | 170 | KAYGL | 314 |
| 125 | QATEAEyLDyDFL | 171 | DHyRA | 315 |
| 126 | QATEyAyLDyDFL | 172 | DHYRA | 316 |
| 127 | QATEyEALDyDFL | 173 | RSyVA | 317 |
| 128 | QATEyEyADyDFL | 174 | RSYVA | 318 |
| 129 | QATEyEyLAyDFL | 175 | INyLA | 319 |
| 130 | QATEyEyLDADFL | 176 | INYLA | 320 |
| 131 | QATEyEyLDyAFL | 177 | TFyIF | 321 |
| 132 | QATEyEyLDyDAL | 178 | TFYIF | 322 |
| 133 | QATEyEyLDyDFA | 179 | HIySR | 323 |
| 134 | QATEYEYLDYDFL | 180 | HIYSR | 324 |
| 135 | QATEyEYLDYDFL | 181 | EIyHS | 325 |
| 136 | QATEYEyLDYDFL | 182 | EIYHS | 326 |
| 137 | QATEYEYLDyDFL | 183 | QQyQP | 327 |
| 138 | QATEyEyLDYDFL | 184 | QQYQP | 328 |
| 139 | QATEyEYLDyDFL | 185 | MFyEA | 329 |
| 140 | QATEYEyLDyDFL | 186 | MFYEA | 330 |
| 141 | QATEyEyLDyDFL | 187 | EVyLE | 331 |
| 142 | QATEyEyLDyDFL | 188 | EVYLE | 332 |
| 143 | QATEyEyLDyDFL | 189 | DAyAN | 333 |
| 144 | QATEyEyLDyDFL | 190 | DAYAN | 334 |

As demonstrated in FIG. 3(A) for the PSG2 antibody, epitope mapping results show that the sulfated tyrosine is essential, and that PSG2 binds to sulfated tyrosine in a wide variety of peptide contexts. The antibody binds in a substantially context-independent manner. Binding that is not substantially different than signals of one or more control antibodies is considered “no specific binding.” To further refine the specificity of PSG2, peptides were constructed with and without sulfated tyrosine. These data appear in FIG. 3(B-D), and show that PSG2 recognizes sulfated tyrosine in a wide variety of unrelated amino acid sequence contexts. The lack of binding to sulfotyrosine in FIG. 3(D) is likely due to steric hindrance as a consequence of the sulfotyrosine's proximity to the cellulose membrane. The substitution analyses of FIGS. 3(B) and (C) indicate that PSG2 disfavors lysine immediately adjacent to, and carboxyl to the sulfated tyrosine (i.e. at the +1 position). Further, a mild to moderate reduction in binding may be associated with a proline or methionine at the +1 position (adjacent on the carboxyl side) as related to the sulfated tyrosine residue. As shown in FIG. 3, the minimal epitope requirement for the PSG2 antibody is Y (Y_{SO4})—the sulfotyrosine is essential.

### Example 7

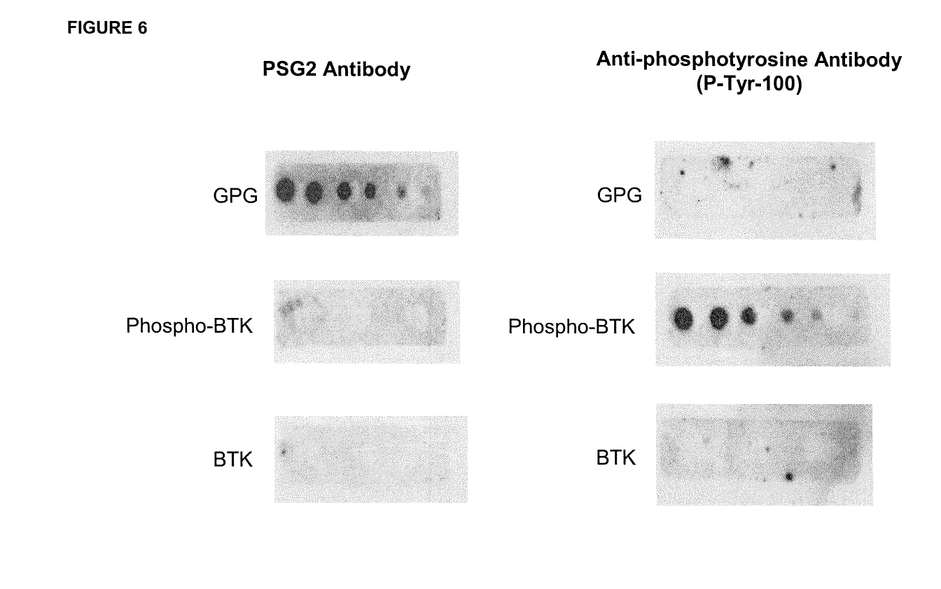

PSG2 is Specific for Sulfotyrosine as Compared to Phosphotyrosine. To compare binding to various peptides, a GPG-290 polypeptide (GPG) or a BTK peptide (BTK) (Tufts peptide) (biotin-βAla-KKVVALYDYMPMN-[OH]) (SEQ ID NO:339), one microliter of 1:3 dilutions of compound was spotted onto P81 phosphocellulose filters (Upstate Cell Signaling #20-134). GPG-290 is a dimeric molecule consisting of the N-terminal 290 amino acids of GPIbα fused to a mutated Fc domain of human IgG1. It contains 3 sulfated tyrosine residues at positions 276, 278, and 279. The BTK peptide is biotin-βAla-KKVVALYDYMPMN-[OH] (SEQ ID NO:339). Phospho-BTK contains 2 phosphorylated tyrosine residues. The starting dilution for GPG-290 was 250 ng/μl and for the BTK peptide the starting dilution was 3 μg/μl. Western Blot analysis was performed as follows: filters were blocked for 1 hour in blocking buffer (TBS+0.1% Tween-20 (TBS/T) and 5% nonfat dry milk). Filters were washed in TBS/T and incubated overnight in primary antibody diluted in TBS/T+0.5% BSA. Washed filters were incubated for 1 hour with secondary antibody diluted in blocking buffer (HRP-mouse anti-human IgG4 to detect PSG-2 and HRP-goat anti-mouse IgG+A+M to detect anti-phospho-tyrosine antibody). HRP signal was detected with the SuperSignal Chemiluminescent Substrate (Pierce) and the filters were exposed to X-ray film. The data are presented in FIG. 6.

### Example 8

Inhibition of Coagulation in Dogs. The effect of sulfotyrosine specific antibody on blood coagulation was measured by bleeding time experiments in dogs. Male mongrel dogs, 10-15 kg in weight, were administered PSG2 (experimental) or IgG.Fc (control) at 1 mg/kg body weight by IV injection.

Bleeding times were measured prior to administration of the PSG2 or IgG.Fc and at 15, 60, and 90 minutes after administration by producing a small incision at the surface of the inner upper lip using an automated spring-loaded device (Simplate R, Organon Teknika). Visual cessation of blood was observed by blotting onto filter paper.

As demonstrated by the data in Table 5, dogs treated with P5G2 had extended bleeding times at 15, 60, and 90 minutes relative to a dog treated with IgG.Fc. No change in heart rate or blood pressure was observed for either experimental or control dogs.

TABLE 5

Bleeding time (min)

| | Baseline | 15 min | 60 min | 90 min |
|---|---|---|---|---|
| PSG2 | | | | |
| Dog #1 | 2.2 | 5.3 | 2.5 | 4 |
| Dog #2 | 2 | 4 | 2.5 | 4 |
| Dog #3 | 1.5 | 7 | 5.8 | 3.5 |
| Average | 1.9 | 5.4 | 4.3 | 3.8 |
| IgG.Fc | | | | |
| Dog #4 | 2.3 | 3 | 1.8 | 2.5 |

### Example 9

Treatment of Sepsis in Humans. An individual having sepsis (e.g., sepsis resulting from a bacterial, viral, fungal, or parasitic infection) is treated with at least one sulfotyrosine specific antibody such as PSG1 or PSG2. The sulfotyrosine specific antibody is administered intravenously or by injection at dosages ranging from approximately 1 μg/kg to 30 mg/kg body weight. The sulfotyrosine specific antibody is optionally administered in combination with one or more antibiotic, antiviral, antifungal, antiparasitic, anti-inflammatory, or blood pressure raising agents. Administration of the anti-sulfotyrosine antibody results in a decrease in blood coagulability and reduction of at least one of the symptoms or clinical indicators of sepsis.

All references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supercede and/or take precedence over any such contradictory material.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. Each numerical parameter should also be construed in light of the number of significant digits and ordinary rounding approaches.

Modifications and variations of this invention can be made without departing from its spirit and scope, as will be apparent to those skilled in the art. The specific embodiments described herein are exemplary and are not meant to be limiting in any way.

What is claimed is:

1. An isolated antibody that specifically binds to sulfated tyrosine in a substantially context-independent manner.

2. The antibody of claim 1, wherein the antibody does not specifically bind to unsulfated tyrosine.

3. The antibody of claim 1, wherein the antibody does not specifically bind to phosphorylated tyrosine.

4. An isolated antibody comprising an amino acid sequence chosen from SEQ ID NO:2, SEQ ID NO:4, SEQ ID NO:6, SEQ ID NO:8, SEQ ID NO:10, SEQ ID NOs:13-18, SEQ ID NOs:19-24, and SEQ ID NOs:12-24, wherein the antibody is capable of specifically binding to sulfated tyrosine in a substantially context-independent manner.

5. The antibody of claim 4, wherein the antibody comprises an scFv fragment.

6. The antibody of claim 4, wherein the antibody specifically binds with an affinity constant greater than 10⁸ M⁻¹.

7. The antibody of claim 1, wherein the antibody is monoclonal.

8. The antibody of claim 1, wherein the antibody is human.

9. The antibody of claim 1, wherein the antibody specifically binds to an Xaa₁-Xaa₂-Tyr-Xaa₃-Xaa₄ peptide but does not specifically bind to the corresponding peptide having an unmodified or phosphorylated tyrosine residue.

10. The antibody of claim 9, wherein Xaa₃ is not lysine.

11. An isolated antibody that specifically binds to sulfated tyrosine, wherein the antibody specifically binds SEQ ID NO:25 and SEQ ID NO:31 but does not specifically bind to SEQ ID NO:26.

12. A pharmaceutical composition comprising the antibody of claim 1.

13. An isolated nucleic acid encoding the antibody of claim 4.

14. An isolated nucleic acid chosen from a nucleic acid comprising:
(a) SEQ ID NOs:1 or 3;
(b) a nucleic acid that encodes SEQ ID NOs: 2, 4, 6, 8, 10, or 12;
(c) a nucleic acid capable of hybridization to a nucleic acid of (a) or (b) under conditions of high stringency and which encodes a polypeptide of the invention; and
(d) a nucleic acid which encodes the same amino acid sequence as a nucleic acid of (c).

15. An expression vector comprising the nucleic acid of claim 14.

16. A host cell comprising the vector of claim 15.

17. A method of making a sulfated tyrosine-specific antibody comprising:
(a) transforming a cell with a DNA construct comprising at least a portion of the nucleic acid of claim 14;
(b) culturing the transformed cell under conditions where an antibody is expressed; and
(c) isolating the antibody.

18. The method of claim 17, wherein the antibody is a monovalent antibody.

19. The method of claim 17, wherein the antibody is a bivalent antibody.

20. A method to produce the antibody of claim 1 that specifically binds to sulfated tyrosine in a substantially context-independent manner comprising:
(a) providing a repertoire of nucleic acids encoding a variable domain that either includes a CDR3 to be replaced or lacks a CDR3 encoding region;
(b) combining the repertoire with a donor nucleic acid encoding an amino acid sequence substantially as set out herein for a V_{H} CDR3 (i.e., H3) such that the donor nucleic acid is inserted into the CDR3 region in the repertoire, so as to provide a product repertoire of nucleic acids encoding a variable domain;
(c) expressing the nucleic acids of said product repertoire; and
(d) selecting an antigen-binding fragment specific for sulfated tyrosine.

21. A method to identify an agent that modulates a protein comprising sulfated tyrosine, comprising
(a) combining the antibody of claim 1 with a ligand, wherein the ligand is a protein comprising a sulfated tyrosine;
(b) detecting modulation of the binding between the ligand and the antibody in the presence and absence of the agent; and
(c) thereby identifying an agent that modulates the protein comprising a sulfated tyrosine.

22. A method to detect a polypeptide comprising sulfated tyrosine in a biological sample, comprising
(a) adding an antibody of claim 1 to a biological sample;
(b) adding a detectable label; and
(c) detecting the amount of the antibody that specifically binds to the sample.

23. A method to detect sulfated proteins or peptides in a biological sample, comprising contacting a biological sample with an antibody of claim 1.

24. A method to quantify the amount of sulfate modified tyrosine in a biological sample, comprising adding an antibody of claim 1 to a biological sample.

25. A kit for detecting a sulfated tyrosine comprising the antibody of claim 1.

26. A method for treating systemic inflammatory response syndrome, comprising administering to an individual an effective dose of the antibody of claim 1.

27. The method of claim 26, wherein the systemic inflammatory response syndrome is sepsis.

28. The method of claim 26, wherein the individual is a mammal.

29. The method of claim 27, wherein the mammal is a human.


SEQUENCE LISTING (340 sequences)

SEQ ID NO: 1 - DNA, 780 bp, Homo sapiens
caggtgcagctgcaggagtccggggctgaggtgaagaagcctggggcctcagtgaaggtctcctgcaagtcttctggatacaccttcaccgcctactatatgcactggctgcgacaggcccctggacaagggcttgagtggatgggctggatcaatcctaacagtggtggcacaaattatgcacagaagtttcagggcagggtcaccttgaccagagacacgtccatcagcacagcctacatggagctgaacagcctgacatctgacgacacggccatgtattactgtgcgagaggaggcccgcgtgtatcttctcgtcccggtataggctactctgactcctggggcaagggaaccctggtcaccgtctcgagtggaggcggcggttcaggcggaggtggctctggcggtggcggaagtgcacagactgtggtgctccaggagccctcactgactgtgtccccaggagggacagtcactctcacctgtgcttccagaattggagcagtcaccagtggtcactatgcaaactggttccagcagaaacctggacaagcacccagggcactgatttatagaacaaacaacaaacagtcctggacccctgcccgattctcaggctccctccttggggacagagctgccctgacactgtcaggtgcgcagcctgaggacgaggctgactattattgcctgctctattatggtggttcttgggtgttcggcggagggaccaagctgaccgtcctaggtgcggccgcacatcatcatcaccatcat

SEQ ID NO: 2 - protein, 260 aa, Homo sapiens
QVQLQESGAEVKKPGASVKVSCKSSGYTFTAYYMHWLRQAPGQGLEWMGWINPNSGGTNYAQKFQGRVTLTRDTSISTAYMELNSLTSDDTAMYYCARGGPRVSSRPGIGYSDSWGKGTLVTVSSGGGGSGGGGSGGGGSAQTVVLQEPSLTVSPGGTVTLTCASRIGAVTSGHYANWFQQKPGQAPRALIYRTNNKQSWTPARFSGSLLGDRAALTLSGAQPEDEADYYCLLYYGGSWVFGGGTKLTVLGAAAHHHHHH

SEQ ID NO: 3 - DNA, 375 bp, Homo sapiens
caggtgcagctgcaggagtccggggctgaggtgaagaagcctggggcctcagtgaaggtctcctgcaagtcttctggatacaccttcaccgcctactatatgcactggctgcgacaggcccctggacaagggcttgagtggatgggctggatcaatcctaacagtggtggcacaaattatgcacagaagtttcagggcagggtcaccttgaccagagacacgtccatcagcacagcctacatggagctgaacagcctgacatctgacgacacggccatgtattactgtgcgagaggaggcccgcgtgtatcttctcgtcccggtataggctactctgactcctggggcaagggaaccctggtcaccgtctcgagt

SEQ ID NO: 4 - protein, 125 aa, Homo sapiens
QVQLQESGAEVKKPGASVKVSCKSSGYTFTAYYMHWLRQAPGQGLEWMGWINPNSGGTNYAQKFQGRVTLTRDTSISTAYMELNSLTSDDTAMYYCARGGPRVSSRPGIGYSDSWGKGTLVTVSS

SEQ ID NO: 5 - DNA, 327 bp, Homo sapiens
cagactgtggtgctccaggagccctcactgactgtgtccccaggagggacagtcactctcacctgtgcttccagaattggagcagtcaccagtggtcactatgcaaactggttccagcagaaacctggacaagcacccagggcactgatttatagaacaaacaacaaacagtcctggacccctgcccgattctcaggctccctccttggggacagagctgccctgacactgtcaggtgcgcagcctgaggacgaggctgactattattgcctgctctattatggtggttcttgggtgttcggcggagggaccaagctgaccgtccta

SEQ ID NO: 6 - protein, 109 aa, Homo sapiens
QTVVLQEPSLTVSPGGTVTLTCASRIGAVTSGHYANWFQQKPGQAPRALIYRTNNKQSWTPARFSGSLLGDRAALTLSGAQPEDEADYYCLLYYGGSWVFGGGTKLTVL

SEQ ID NO: 7 - DNA, 783 bp, Homo sapiens
gaggtgcagctggtggagtctgggggagacttggtacagcctggggagtccctgggactctcctgtgtaggctctgaattcaactttggcagttatggcatgacctgggtccgccaggctccagggaaggggctggagtgggtctcaagtattagtagtgctggtaaaacattctacgcagactccgtgaagggccgattcaccatctctagagacaattccaagaacacggtgtttctgcaaatgaacaacctgagagtcgaggacacggccgtttattactgtgcgaaggggcgtggacacagctatgggcgacctctggcctcctggggccgggggacaatggtcaccgtctcgagtggaggcggcggttcaggcggaggtggctctggcggtggcggaagtgcacaggctgtgctgactcagccgtcttccctctctgcatctcctggagcatcagccagtctcacctgcaccttacgcagtggcatcgatgttggtccccacagaatatactggttccagcagaagccagggagtactccccagtatctcctgaggtacaaatcagactcagatacgcagcagggctctggagtccccagccgattctctggatccaaagatgcttcggccaatgcagggattttactcatctctgggctccagtctgaggatgaggccgactattattgtatgatttggcacagcagcgcttgggtgttcggcggagggaccaagctgaccgtcctaggtgcggccgcacatcatcatcaccatcat

SEQ ID NO: 8 - protein, 261 aa, Homo sapiens
EVQLVESGGDLVQPGESLGLSCVGSEFNFGSYGMTWVRQAPGKGLEWVSSISSAGKTFYADSVKGRFTISRDNSKNTVFLQMNNLRVEDTAVYYCAKGRGHSYGRPLASWGRGTMVTVSSGGGGSGGGGSGGGGSAQAVLTQPSSLSASPGASASLTCTLRSGIDVGPHRIYWFQQKPGSTPQYLLRYKSDSDTQQGSGVPSRFSGSKDASANAGILLISGLQSEDEADYYCMIWHSSAWVFGGGTKLTVLGAAAHHHHHH

SEQ ID NO: 9 - DNA, 360 bp, Homo sapiens
gaggtgcagctggtggagtctgggggagacttggtacagcctggggagtccctgggactctcctgtgtaggctctgaattcaactttggcagttatggcatgacctgggtccgccaggctccagggaaggggctggagtgggtctcaagtattagtagtgctggtaaaacattctacgcagactccgtgaagggccgattcaccatctctagagacaattccaagaacacggtgtttctgcaaatgaacaacctgagagtcgaggacacggccgtttattactgtgcgaaggggcgtggacacagctatgggcgacctctggcctcctggggccgggggacaatggtcaccgtctcgagt

SEQ ID NO: 10 - protein, 120 aa, Homo sapiens
EVQLVESGGDLVQPGESLGLSCVGSEFNFGSYGMTWVRQAPGKGLEWVSSISSAGKTFYADSVKGRFTISRDNSKNTVFLQMNNLRVEDTAVYYCAKGRGHSYGRPLASWGRGTMVTVSS

SEQ ID NO: 11 - DNA, 345 bp, Homo sapiens
  modified_base (41): a, c, g, t, unknown, or other
caggctgtgctgactcagccgtcttccctctctgcatctcntggagcatcagccagtctcacctgcaccttacgcagtggcatcgatgttggtccccacagaatatactggttccagcagaagccagggagtactccccagtatctcctgaggtacaaatcagactcagatacgcagcagggctctggagtccccagccgattctctggatccaaagatgcttcggccaatgcagggattttactcatctctgggctccagtctgaggatgaggccgactattattgtatgatttggcacagcagcgcttgggtgttcggcggagggaccaagctgaccgtccta

SEQ ID NO: 12 - protein, 115 aa, Homo sapiens
  MOD_RES (14): Variable amino acid
QAVLTQPSSLSASXGASASLTCTLRSGIDVGPHRIYWFQQKPGSTPQYLLRYKSDSDTQQGSGVPSRFSGSKDASANAGILLISGLQSEDEADYYCMIWHSSAWVFGGGTKLTVL

SEQ ID NO: 13 - protein, 5 aa, Homo sapiens
AYYMH

SEQ ID NO: 14 - protein, 17 aa, Homo sapiens
WINPNSGGTNYAQKFQG

SEQ ID NO: 15 - protein, 16 aa, Homo sapiens
GGPRVSSRPGIGYSDS

SEQ ID NO: 16 - protein, 14 aa, Homo sapiens
ASRIGAVTSGHYAN

SEQ ID NO: 17 - protein, 7 aa, Homo sapiens
RTNNKQS

SEQ ID NO: 18 - protein, 9 aa, Homo sapiens
LLYYGGSWV

SEQ ID NO: 19 - protein, 5 aa, Homo sapiens
SYGMT

SEQ ID NO: 20 - protein, 16 aa, Homo sapiens
SISSAGKTFYADSVKG

SEQ ID NO: 21 - protein, 12 aa, Homo sapiens
GRGHSYGRPLAS

SEQ ID NO: 22 - protein, 14 aa, Homo sapiens
TLRSGIDVGPHRIY

SEQ ID NO: 23 - protein, 10 aa, Homo sapiens
KSDSDTQQGS

SEQ ID NO: 24 - protein, 9 aa, Homo sapiens
MIWHSSAWV

SEQ ID NO: 25 - protein, 13 aa, Homo sapiens
  MOD_RES (5): Sulfated Tyr
  MOD_RES (7): Sulfated Tyr
  MOD_RES (10): Sulfated Tyr
QATEYEYLDYDFL

SEQ ID NO: 26 - protein, 13 aa, Homo sapiens
QATEYEYLDYDFL

SEQ ID NO: 27 - protein, 10 aa, Homo sapiens
DLYDYYPEED

SEQ ID NO: 28 - protein, 5 aa, Artificial Sequence
  Description of Artificial Sequence Synthetic peptide
  MOD_RES (3): Sulfated Tyr
LDYDF

SEQ ID NO: 29 - protein, 5 aa, Artificial Sequence
  Description of Artificial Sequence Synthetic peptide
  MOD_RES (3): Sulfated Tyr
TEYER

SEQ ID NO: 30 - protein, 28 aa, Homo sapiens
  MOD_RES (5): Sulfated Tyr
  MOD_RES (7): Sulfated Tyr
  MOD_RES (10): Sulfated Tyr
QATEYEYLDYDFLPETEPPRPMMDDDDK

SEQ ID NO: 31 - protein, 10 aa, Homo sapiens
  MOD_RES (3): Sulfated Tyr
  MOD_RES (5)..(6): Sulfated Tyr
DLYDYYPEED

SEQ ID NO: 32 - protein, 10 aa, Mus sp.
  MOD_RES (4): Sulfated Tyr
  MOD_RES (6): Sulfated Tyr
DPDYTYNTDP

SEQ ID NO: 33 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - protein, 108 aa, Homo sapiens
HMTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 19 aa, Homo sapiens
QATEYEYLDYDFLPETEPP

SEQ ID NO: 38 - protein, 28 aa, Artificial Sequence
  Description of Artificial Sequence Synthetic peptide
QATEYEYLDYDFLPETEPPRPMMDDDDK

SEQ ID NO: 39 - protein, 28 aa, Artificial Sequence
  Description of Artificial Sequence Synthetic peptide
  MOD_RES (5): Sulfated Tyr
QATEYEYLDYDFLPETEPPRPMMDDDDK

SEQ ID NO: 40 - protein, 28 aa, Artificial Sequence
  Description of Artificial Sequence Synthetic peptide
  MOD_RES (7): Sulfated Tyr
QATEYEYLDYDFLPETEPPRPMMDDDDK

SEQ ID NO: 41 - protein, 28 aa, Artificial Sequence
  Description of Artificial Sequence Synthetic peptide
  MOD_RES (10): Sulfated Tyr
QATEYEYLDYDFLPETEPPRPMMDDDDK

SEQ ID NO: 42 - protein, 28 aa, Artificial Sequence
  Description of Artificial Sequence Synthetic peptide
  MOD_RES (7): Sulfated Tyr
  MOD_RES (10): Sulfated Tyr
QATEYEYLDYDFLPETEPPRPMMDDDDK

SEQ ID NO: 43 - protein, 28 aa, Artificial Sequence
  Description of Artificial Sequence Synthetic peptide
  MOD_RES (5): Sulfated Tyr
  MOD_RES (10): Sulfated Tyr
QATEYEYLDYDFLPETEPPRPMMDDDDK

SEQ ID NO: 44 - protein, 28 aa, Artificial Sequence
  Description of Artificial Sequence Synthetic peptide
  MOD_RES (5): Sulfated Tyr
  MOD_RES (7): Sulfated Tyr
QATEYEYLDYDFLPETEPPRPMMDDDDK

SEQ ID NO: 45 - protein, 45 aa, Mus sp.
  MOD_RES (13): Sulfated Tyr
  MOD_RES (15): Sulfated Tyr
QVVGDDDFEDPDYTYNTDPPELLKNVTNTVAAHPELPTTVVMLER

SEQ ID NO: 46 - protein, 531 aa, Homo sapiens
MPLLLLLLLLPSPLHPHPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVKAMTSNVASVQCDNSDKFPVYKYPGKGCPTLGDEGDTDLYDYYPEEDTEGDKVRPHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 48 - protein, 13 aa, Artificial Sequence
  Description of Artificial Sequence Synthetic peptide
QATEYEYLDYDFL

SEQ ID NO: 49 - protein, 13 aa, Artificial Sequence
  Description of Artificial Sequence Synthetic peptide
  MOD_RES (5): Sulfated Tyr
QATEYEYLDYDFL

SEQ ID NO: 50 - protein, 13 aa, Artificial Sequence
  Description of Artificial Sequence Synthetic peptide
  MOD_RES (7): Sulfated Tyr
QATEYEYLDYDFL

SEQ ID NO: 51 - protein, 13 aa, Artificial Sequence
  Description of Artificial Sequence Synthetic peptide
  MOD_RES (10): Sulfated Tyr
QATEYEYLDYDFL

SEQ ID NO: 52 - protein, 13 aa, Artificial Sequence
  Description of Artificial Sequence Synthetic peptide
  MOD_RES (5): Sulfated Tyr
  MOD_RES (7): Sulfated Tyr
QATEYEYLDYDFL

SEQ ID NO: 53 - protein, 13 aa, Artificial Sequence
  Description of Artificial Sequence Synthetic peptide
  MOD_RES (5): Sulfated Tyr
  MOD_RES (10): Sulfated Tyr
QATEYEYLDYDFL

SEQ ID NO: 54 - protein, 13 aa, Artificial Sequence
  Description of Artificial Sequence Synthetic peptide
  MOD_RES (7): Sulfated Tyr
  MOD_RES (10): Sulfated Tyr
QATEYEYLDYDFL

SEQ ID NO: 55 - protein, 12 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDF

SEQ ID NO: 56 - protein, 11 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYD

SEQ ID NO: 57 - protein, 12 aa, Artificial Sequence
  Description of Artificial Sequence Synthetic peptide
  MOD_RES (4): Sulfated Tyr
  MOD_RES (6): Sulfated Tyr
  MOD_RES (9): Sulfated Tyr
ATEYEYLDYDFL

SEQ ID NO: 58 - protein, 11 aa, Artificial Sequence
  Description of Artificial Sequence Synthetic peptide
  MOD_RES (4): Sulfated Tyr
  MOD_RES (6): Sulfated Tyr
  MOD_RES (9): Sulfated Tyr
ATEYEYLDYDF

SEQ ID NO: 59 - protein, 10 aa, Artificial Sequence
  Description of Artificial Sequence Synthetic peptide
  MOD_RES (4): Sulfated Tyr
  MOD_RES (6): Sulfated Tyr
  MOD_RES (9): Sulfated Tyr
ATEYEYLDYD

SEQ ID NO: 60 - protein, 11 aa, Artificial Sequence
  Description of Artificial Sequence Synthetic peptide
  MOD_RES (3): Sulfated Tyr
  MOD_RES (5): Sulfated Tyr
  MOD_RES (8): Sulfated Tyr
TEYEYLDYDFL

SEQ ID NO: 61 - protein, 10 aa, Artificial Sequence
  Description of Artificial Sequence Synthetic peptide
  MOD_RES (3): Sulfated Tyr
  MOD_RES (5): Sulfated Tyr
  MOD_RES (8): Sulfated Tyr
TEYEYLDYDF

SEQ ID NO: 62 - protein, 9 aa, Artificial Sequence
  Description of Artificial Sequence Synthetic peptide
  MOD_RES (3): Sulfated Tyr
  MOD_RES (5): Sulfated Tyr
  MOD_RES (8): Sulfated Tyr
TEYEYLDYD

SEQ ID NO: 63 - protein, 10 aa, Artificial Sequence
  Description of Artificial Sequence Synthetic peptide
  MOD_RES (2): Sulfated Tyr
  MOD_RES (4): Sulfated Tyr
  MOD_RES (7): Sulfated Tyr
EYEYLDYDFL

SEQ ID NO: 64 - protein, 9 aa, Artificial Sequence
  Description of Artificial Sequence Synthetic peptide
  MOD_RES (2): Sulfated Tyr
  MOD_RES (4): Sulfated Tyr
  MOD_RES (7): Sulfated Tyr
EYEYLDYDF

SEQ ID NO: 65 - protein, 8 aa, Artificial Sequence
  Description of Artificial Sequence Synthetic peptide
  MOD_RES (2): Sulfated Tyr
  MOD_RES (4): Sulfated Tyr
  MOD_RES (7): Sulfated Tyr
EYEYLDYD

SEQ ID NO: 66 - protein, 12 aa, Artificial Sequence
  Description of Artificial Sequence Synthetic peptide
  MOD_RES (5): Sulfated Tyr
QATEYEYLDYDF

SEQ ID NO: 67 - protein, 11 aa, Artificial Sequence
  Description of Artificial Sequence Synthetic peptide
  MOD_RES (5): Sulfated Tyr
QATEYEYLDYD

SEQ ID NO: 68 - protein, 12 aa, Artificial Sequence
  Description of Artificial Sequence Synthetic peptide
  MOD_RES (4): Sulfated Tyr
ATEYEYLDYDFL

SEQ ID NO: 69 - protein, 11 aa, Artificial Sequence
  Description of Artificial Sequence Synthetic peptide
  MOD_RES (4): Sulfated Tyr
ATEYEYLDYDF

SEQ ID NO: 70 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 71 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 72 - protein, 10 aa, Artificial Sequence
  Description of Artificial Sequence Synthetic peptide
  MOD_RES (4): Sulfated Tyr
ATEYEYLDYD

SEQ ID NO: 73 - protein, 11 aa, Artificial Sequence
  Description of Artificial Sequence Synthetic peptide
  MOD_RES (3): Sulfated Tyr
TEYEYLDYDFL

SEQ ID NO: 74 - protein, 10 aa, Artificial Sequence
  Description of Artificial Sequence Synthetic peptide
  MOD_RES (3): Sulfated Tyr
TEYEYLDYDF

SEQ ID NO: 75 - protein, 9 aa, Artificial Sequence
  Description of Artificial Sequence Synthetic peptide
  MOD_RES (3): Sulfated Tyr
TEYEYLDYD

SEQ ID NO: 76 - protein, 10 aa, Artificial Sequence
  Description of Artificial Sequence Synthetic peptide
  MOD_RES (2): Sulfated Tyr
EYEYLDYDFL

SEQ ID NO: 77 - protein, 9 aa, Artificial Sequence
  Description of Artificial Sequence Synthetic peptide
  MOD_RES (2): Sulfated Tyr
EYEYLDYDF

SEQ ID NO: 78 - protein, 8 aa, Artificial Sequence
  Description of Artificial Sequence Synthetic peptide
  MOD_RES (2): Sulfated Tyr
EYEYLDYD

SEQ ID NO: 79 - protein, 12 aa, Artificial Sequence
  Description of Artificial Sequence Synthetic peptide
  MOD_RES (7): Sulfated Tyr
QATEYEYLDYDF

SEQ ID NO: 80 - protein, 11 aa, Artificial Sequence
  Description of Artificial Sequence Synthetic peptide
  MOD_RES (7): Sulfated Tyr
QATEYEYLDYD

SEQ ID NO: 81 - protein, 12 aa, Artificial Sequence
  Description of Artificial Sequence Synthetic peptide
  MOD_RES (6): Sulfated Tyr
ATEYEYLDYDFL

SEQ ID NO: 82 - protein, 11 aa, Artificial Sequence
  Description of Artificial Sequence Synthetic peptide
  MOD_RES (6): Sulfated Tyr
ATEYEYLDYDF

SEQ ID NO: 83 - protein, 10 aa, Artificial Sequence
  Description of Artificial Sequence Synthetic peptide
  MOD_RES (6): Sulfated Tyr
ATEYEYLDYD

SEQ ID NO: 84 - protein, 11 aa, Artificial Sequence
  Description of Artificial Sequence Synthetic peptide
  MOD_RES (5): Sulfated Tyr
TEYEYLDYDFL

SEQ ID NO: 85 - protein, 10 aa, Artificial Sequence
  Description of Artificial Sequence Synthetic peptide
  MOD_RES (5): Sulfated Tyr
TEYEYLDYDF

SEQ ID NO: 86 - protein, 9 aa, Artificial Sequence
  Description of Artificial Sequence Synthetic peptide
  MOD_RES (5): Sulfated Tyr
TEYEYLDYD

SEQ ID NO: 87 - protein, 10 aa, Artificial Sequence
  Description of Artificial Sequence Synthetic peptide
  MOD_RES (4): Sulfated Tyr
EYEYLDYDFL

SEQ ID NO: 88 - protein, 9 aa, Artificial Sequence
  Description of Artificial Sequence Synthetic peptide
  MOD_RES (4): Sulfated Tyr
EYEYLDYDF

SEQ ID NO: 89 - protein, 8 aa, Artificial Sequence
  Description of Artificial Sequence Synthetic peptide
  MOD_RES (4): Sulfated Tyr
EYEYLDYD

SEQ ID NO: 90 - protein, 12 aa, Artificial Sequence
  Description of Artificial Sequence Synthetic peptide
  MOD_RES (10): Sulfated Tyr
QATEYEYLDYDF

SEQ ID NO: 91 - protein, 11 aa, Artificial Sequence
  Description of Artificial Sequence Synthetic peptide
  MOD_RES (10): Sulfated Tyr
QATEYEYLDYD

SEQ ID NO: 92 - protein, 12 aa, Artificial Sequence
  Description of Artificial Sequence Synthetic peptide
  MOD_RES (9): Sulfated Tyr
ATEYEYLDYDFL

SEQ ID NO: 93 - protein, 11 aa, Artificial Sequence
  Description of Artificial Sequence Synthetic peptide
  MOD_RES (9): Sulfated Tyr
ATEYEYLDYDF

SEQ ID NO: 94 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 95 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 96 - protein, 10 aa, Artificial Sequence
  Description of Artificial Sequence Synthetic peptide
  MOD_RES (9): Sulfated Tyr
ATEYEYLDYD

SEQ ID NO: 97 - protein, 11 aa, Artificial Sequence
  Description of Artificial Sequence Synthetic peptide
  MOD_RES (8): Sulfated Tyr
TEYEYLDYDFL

SEQ ID NO: 98 - protein, 10 aa, Artificial Sequence
  Description of Artificial Sequence Synthetic peptide
  MOD_RES (8): Sulfated Tyr
TEYEYLDYDF

SEQ ID NO: 99 - protein, 9 aa, Artificial Sequence
  Description of Artificial Sequence Synthetic peptide
  MOD_RES (8): Sulfated Tyr
TEYEYLDYD

SEQ ID NO: 100 - protein, 10 aa, Artificial Sequence
  Description of Artificial Sequence Synthetic peptide
  MOD_RES (7): Sulfated Tyr
EYEYLDYDFL

SEQ ID NO: 101 - protein, 9 aa, Artificial Sequence
  Description of Artificial Sequence Synthetic peptide
  MOD_RES (7): Sulfated Tyr
EYEYLDYDF

SEQ ID NO: 102 - protein, 8 aa, Artificial Sequence
  Description of Artificial Sequence Synthetic peptide
  MOD_RES (7): Sulfated Tyr
EYEYLDYD

SEQ ID NO: 103 - protein, 12 aa, Artificial Sequence
  Description of Artificial Sequence Synthetic peptide
  MOD_RES (5): Sulfated Tyr
  MOD_RES (10): Sulfated Tyr
QATEYEYLDYDF

SEQ ID NO: 104 - protein, 11 aa, Artificial Sequence
  Description of Artificial Sequence Synthetic peptide
  MOD_RES (5): Sulfated Tyr
  MOD_RES (10): Sulfated Tyr
QATEYEYLDYD

SEQ ID NO: 105 - protein, 12 aa, Artificial Sequence
  Description of Artificial Sequence Synthetic peptide
  MOD_RES (4): Sulfated Tyr
  MOD_RES (9): Sulfated Tyr
ATEYEYLDYDFL

SEQ ID NO: 106 - protein, 11 aa, Artificial Sequence
  Description of Artificial Sequence Synthetic peptide
  MOD_RES (4): Sulfated Tyr
  MOD_RES (9): Sulfated Tyr
ATEYEYLDYDF

SEQ ID NO: 107 - protein, 10 aa, Artificial Sequence
  Description of Artificial Sequence Synthetic peptide
  MOD_RES (4): Sulfated Tyr
  MOD_RES (9): Sulfated Tyr
ATEYEYLDYD

SEQ ID NO: 108 - protein, 11 aa, Artificial Sequence
  Description of Artificial Sequence Synthetic peptide
  MOD_RES (3): Sulfated Tyr
  MOD_RES (8): Sulfated Tyr
TEYEYLDYDFL

SEQ ID NO: 109 - protein, 10 aa, Artificial Sequence
  Description of Artificial Sequence Synthetic peptide
  MOD_RES (3): Sulfated Tyr
  MOD_RES (8): Sulfated Tyr
TEYEYLDYDF

SEQ ID NO: 110 - protein, 9 aa, Artificial Sequence
  Description of Artificial Sequence Synthetic peptide
  MOD_RES (3): Sulfated Tyr
  MOD_RES (8): Sulfated Tyr
TEYEYLDYD

SEQ ID NO: 111 - protein, 10 aa, Artificial Sequence
  Description of Artificial Sequence Synthetic peptide
  MOD_RES (2): Sulfated Tyr
  MOD_RES (7): Sulfated Tyr
EYEYLDYDFL

SEQ ID NO: 112 - protein, 9 aa, Artificial Sequence
  Description of Artificial Sequence Synthetic peptide
  MOD_RES (2): Sulfated Tyr
  MOD_RES (7): Sulfated Tyr
EYEYLDYDF

SEQ ID NO: 113 - protein, 8 aa, Artificial Sequence
  Description of Artificial Sequence Synthetic peptide
  MOD_RES (2): Sulfated Tyr
  MOD_RES (7): Sulfated Tyr
EYEYLDYD

SEQ ID NO: 114 - protein, 12 aa, Artificial Sequence
  Description of Artificial Sequence Synthetic peptide
  MOD_RES (7): Sulfated Tyr
  MOD_RES (10): Sulfated Tyr
QATEYEYLDYDF

SEQ ID NO: 115 - protein, 11 aa, Artificial Sequence
  Description of Artificial Sequence Synthetic peptide
  MOD_RES (7): Sulfated Tyr
  MOD_RES (10): Sulfated Tyr
QATEYEYLDYD

SEQ ID NO: 116 - protein, 12 aa, Artificial Sequence
  Description of Artificial Sequence Synthetic peptide
  MOD_RES (6): Sulfated Tyr
  MOD_RES (9): Sulfated Tyr
ATEYEYLDYDFL

SEQ ID NO: 117 - protein, 11 aa, Artificial Sequence
  Description of Artificial Sequence Synthetic peptide
  MOD_RES (6): Sulfated Tyr
  MOD_RES (9): Sulfated Tyr
ATEYEYLDYDF

SEQ ID NO: 118 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 119 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 120 - protein, 10 aa, Artificial Sequence
  Description of Artificial Sequence Synthetic peptide
  MOD_RES (6): Sulfated Tyr
  MOD_RES (9): Sulfated Tyr
ATEYEYLDYD

SEQ ID NO: 121 - protein, 11 aa, Artificial Sequence
  Description of Artificial Sequence Synthetic peptide
  MOD_RES (5): Sulfated Tyr
  MOD_RES (8): Sulfated Tyr
TEYEYLDYDFL

SEQ ID NO: 122 - protein, 10 aa, Artificial Sequence
  Description of Artificial Sequence Synthetic peptide
  MOD_RES (5): Sulfated Tyr
  MOD_RES (8): Sulfated Tyr
TEYEYLDYDF

SEQ ID NO: 123 - protein, 9 aa, Artificial Sequence
  Description of Artificial Sequence Synthetic peptide
  MOD_RES (5): Sulfated Tyr
  MOD_RES (8): Sulfated Tyr
TEYEYLDYD

SEQ ID NO: 124 - protein, 10 aa, Artificial Sequence
  Description of Artificial Sequence Synthetic peptide
  MOD_RES (4): Sulfated Tyr
  MOD_RES (7): Sulfated Tyr
EYEYLDYDFL

SEQ ID NO: 125 - protein, 9 aa, Artificial Sequence
  Description of Artificial Sequence Synthetic peptide
  MOD_RES (4): Sulfated Tyr
  MOD_RES (7): Sulfated Tyr
EYEYLDYDF

SEQ ID NO: 126 - protein, 8 aa, Artificial Sequence
  Description of Artificial Sequence Synthetic peptide
  MOD_RES (4): Sulfated Tyr
  MOD_RES (7): Sulfated Tyr
EYEYLDYD

SEQ ID NO: 127 - protein, 12 aa, Artificial Sequence
  Description of Artificial Sequence Synthetic peptide
  MOD_RES (5): Sulfated Tyr
  MOD_RES (7): Sulfated Tyr
QATEYEYLDYDF

SEQ ID NO: 128 - protein, 11 aa, Artificial Sequence
  Description of Artificial Sequence Synthetic peptide
  MOD_RES (5): Sulfated Tyr
  MOD_RES (7): Sulfated Tyr
QATEYEYLDYD

SEQ ID NO: 129 - protein, 12 aa, Artificial Sequence
  Description of Artificial Sequence Synthetic peptide
  MOD_RES (4): Sulfated Tyr
  MOD_RES (6): Sulfated Tyr
ATEYEYLDYDFL

SEQ ID NO: 130 - protein, 11 aa, Artificial Sequence
  Description of Artificial Sequence Synthetic peptide
  MOD_RES (4): Sulfated Tyr
  MOD_RES (6): Sulfated Tyr
ATEYEYLDYDF

SEQ ID NO: 131 - protein, 10 aa, Artificial Sequence
  Description of Artificial Sequence Synthetic peptide
  MOD_RES (4): Sulfated Tyr
  MOD_RES (6): Sulfated Tyr
ATEYEYLDYD

SEQ ID NO: 132 - protein, 11 aa, Artificial Sequence
  Description of Artificial Sequence Synthetic peptide
  MOD_RES (3): Sulfated Tyr
  MOD_RES (5): Sulfated Tyr
TEYEYLDYDFL

SEQ ID NO: 133 - protein, 10 aa, Artificial Sequence
  Description of Artificial Sequence Synthetic peptide
  MOD_RES (3): Sulfated Tyr
  MOD_RES (5): Sulfated Tyr
TEYEYLDYDF

SEQ ID NO: 134 - protein, 9 aa, Artificial Sequence
  Description of Artificial Sequence Synthetic peptide
  MOD_RES (3): Sulfated Tyr
  MOD_RES (5): Sulfated Tyr
TEYEYLDYD

SEQ ID NO: 135 - protein, 10 aa, Artificial Sequence
  Description of Artificial Sequence Synthetic peptide
  MOD_RES (2): Sulfated Tyr
  MOD_RES (4): Sulfated Tyr
EYEYLDYDFL

SEQ ID NO: 136 - protein, 9 aa, Artificial Sequence
  Description of Artificial Sequence Synthetic peptide
  MOD_RES (2): Sulfated Tyr
  MOD_RES (4): Sulfated Tyr
EYEYLDYDF

SEQ ID NO: 137 - protein, 8 aa, Artificial Sequence
  Description of Artificial Sequence Synthetic peptide
  MOD_RES (2): Sulfated Tyr
  MOD_RES (4): Sulfated Tyr
EYEYLDYD

SEQ ID NO: 138 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 139 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 140 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 141 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 142 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 143 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 144 - protein, 13 aa, Artificial Sequence
  Description of Artificial Sequence Synthetic peptide
  MOD_RES (8): Sulfated Tyr
  MOD_RES (10): Sulfated Tyr
DDFEDPDYTYNTD

SEQ ID NO: 145 - protein, 13 aa, Artificial Sequence
  Description of Artificial Sequence Synthetic peptide
DDFEDPDYTYNTD

SEQ ID NO: 146 - protein, 13 aa, Artificial Sequence
  Description of Artificial Sequence Synthetic peptide
  MOD_RES (8): Sulfated Tyr
DDFEDPDYTYNTD

SEQ ID NO: 147 - protein, 13 aa, Artificial Sequence
  Description of Artificial Sequence Synthetic peptide
  MOD_RES (10): Sulfated Tyr
DDFEDPDYTYNTD

SEQ ID NO: 148 - protein, 13 aa, Artificial Sequence
  Description of Artificial Sequence Synthetic peptide
  MOD_RES (4): Sulfated Tyr
  MOD_RES (7): Sulfated Tyr
  MOD_RES (10): Sulfated Tyr
DFEYPDYSVYGTD

SEQ ID NO: 149 - protein, 13 aa, Artificial Sequence
  Description of Artificial Sequence Synthetic peptide
DFEYPDYSVYGTD

SEQ ID NO: 150 - protein, 13 aa, Artificial Sequence
  Description of Artificial Sequence Synthetic peptide
  MOD_RES (4): Sulfated Tyr
DFEYPDYSVYGTD

SEQ ID NO: 151 - protein, 13 aa, Artificial Sequence
  Description of Artificial Sequence Synthetic peptide
  MOD_RES (7): Sulfated Tyr
DFEYPDYSVYGTD

SEQ ID NO: 152 - protein, 13 aa, Artificial Sequence
  Description of Artificial Sequence Synthetic peptide
  MOD_RES (10): Sulfated Tyr
DFEYPDYSVYGTD

SEQ ID NO: 153 - protein, 13 aa, Artificial Sequence
  Description of Artificial Sequence Synthetic peptide
  MOD_RES (4): Sulfated Tyr
  MOD_RES (7): Sulfated Tyr
DFEYPDYSVYGTD

SEQ ID NO: 154 - protein, 13 aa, Artificial Sequence
  Description of Artificial Sequence Synthetic peptide
  MOD_RES (4): Sulfated Tyr
  MOD_RES (10): Sulfated Tyr
DFEYPDYSVYGTD

SEQ ID NO: 155 - protein, 13 aa, Artificial Sequence
  Description of Artificial Sequence Synthetic peptide
  MOD_RES (7): Sulfated Tyr
  MOD_RES (10): Sulfated Tyr
DFEYPDYSVYGTD

SEQ ID NO: 156 - protein, 13 aa, Artificial Sequence
  Description of Artificial Sequence Synthetic peptide
  MOD_RES (6): Sulfated Tyr
  MOD_RES (8)..(9): Sulfated Tyr
GDTDLYDYYPEED

SEQ ID NO: 157 - protein, 13 aa, Artificial Sequence
  Description of Artificial Sequence Synthetic peptide
GDTDLYDYYPEED

SEQ ID NO: 158 - protein, 13 aa, Artificial Sequence
  Description of Artificial Sequence Synthetic peptide
  MOD_RES (6): Sulfated Tyr
GDTDLYDYYPEED

SEQ ID NO: 159 - protein, 13 aa, Artificial Sequence
  Description of Artificial Sequence Synthetic peptide
  MOD_RES (8): Sulfated Tyr
GDTDLYDYYPEED

SEQ ID NO: 160 - protein, 13 aa, Artificial Sequence
  Description of Artificial Sequence Synthetic peptide
  MOD_RES (9): Sulfated Tyr
GDTDLYDYYPEED

SEQ ID NO: 161 - protein, 13 aa, Artificial Sequence
  Description of Artificial Sequence Synthetic peptide
  MOD_RES (6): Sulfated Tyr
  MOD_RES (8): Sulfated Tyr
GDTDLYDYYPEED

SEQ ID NO: 162 - protein, 13 aa, Artificial Sequence
  Description of Artificial Sequence Synthetic peptide
  MOD_RES (6): Sulfated Tyr
  MOD_RES (9): Sulfated Tyr
GDTDLYDYYPEED

SEQ ID NO: 163 - protein, 13 aa, Artificial Sequence
  Description of Artificial Sequence Synthetic peptide
  MOD_RES (8)..(9): Sulfated Tyr
GDTDLYDYYPEED

SEQ ID NO: 164 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 165 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 166 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 167 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 168 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
AATEYEYLDYDFL

SEQ ID NO: 169 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QAAEYEYLDYDFL

SEQ ID NO: 170 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATAYEYLDYDFL

SEQ ID NO: 171 - protein, 13 aa, Artificial Sequence
  Description of Artificial Sequence Synthetic peptide
  MOD_RES (7): Sulfated Tyr
  MOD_RES (10): Sulfated Tyr
QATEAEYLDYDFL

SEQ ID NO: 172 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYAYLDYDFL

SEQ ID NO: 173 - protein, 13 aa, Artificial Sequence
  Description of Artificial Sequence Synthetic peptide
  MOD_RES (5): Sulfated Tyr
  MOD_RES (10): Sulfated Tyr
QATEYEALDYDFL

SEQ ID NO: 174 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYADYDFL

SEQ ID NO: 175 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLAYDFL

SEQ ID NO: 176 - protein, 13 aa, Artificial Sequence
  Description of Artificial Sequence Synthetic peptide
  MOD_RES (5): Sulfated Tyr
  MOD_RES (7): Sulfated Tyr
QATEYEYLDADFL

SEQ ID NO: 177 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYAFL

SEQ ID NO: 178 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDAL

SEQ ID NO: 179 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFA

SEQ ID NO: 180 - protein, 13 aa, Artificial Sequence
  Description of Artificial Sequence Synthetic peptide
QATEYEYLDYDFL

SEQ ID NO: 181 - protein, 13 aa, Artificial Sequence
  Description of Artificial Sequence Synthetic peptide
  MOD_RES (5): Sulfated Tyr
QATEYEYLDYDFL

SEQ ID NO: 182 - protein, 13 aa, Artificial Sequence
  Description of Artificial Sequence Synthetic peptide
  MOD_RES (7): Sulfated Tyr
QATEYEYLDYDFL

SEQ ID NO: 183 - protein, 13 aa, Artificial Sequence
  Description of Artificial Sequence Synthetic peptide
  MOD_RES (10): Sulfated Tyr
QATEYEYLDYDFL

SEQ ID NO: 184 - protein, 13 aa, Artificial Sequence
  Description of Artificial Sequence Synthetic peptide
  MOD_RES (5): Sulfated Tyr
  MOD_RES (7): Sulfated Tyr
QATEYEYLDYDFL

SEQ ID NO: 185 - protein, 13 aa, Artificial Sequence
  Description of Artificial Sequence Synthetic peptide
  MOD_RES (5): Sulfated Tyr
  MOD_RES (10): Sulfated Tyr
QATEYEYLDYDFL

SEQ ID NO: 186 - protein, 13 aa, Artificial Sequence
  Description of Artificial Sequence Synthetic peptide
  MOD_RES (7): Sulfated Tyr
  MOD_RES (10): Sulfated Tyr
QATEYEYLDYDFL

SEQ ID NO: 187 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 188 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 189 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 190 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 191 - protein, 5 aa, Artificial Sequence
  Description of Artificial Sequence Synthetic peptide
  MOD_RES (3): Sulfated Tyr
AAYAA

SEQ ID NO: 192 - protein, 5 aa, Artificial Sequence
  Description of Artificial Sequence Synthetic peptide
AAYAA

SEQ ID NO: 193 - protein, 3 aa, Artificial Sequence
  Description of Artificial Sequence Synthetic peptide
  MOD_RES (2): Sulfated Tyr
AYA

SEQ ID NO: 194 - protein, 3 aa, Artificial Sequence
  Description of Artificial Sequence Synthetic peptide
AYA

SEQ ID NO: 195 - protein, 2 aa, Artificial Sequence
  Description of Artificial Sequence Synthetic peptide
  MOD_RES (1): Sulfated Tyr
YA

SEQ ID NO: 196 - protein, 2 aa, Artificial Sequence
  Description of Artificial Sequence Synthetic peptide
YA

SEQ ID NO: 197 - protein, 2 aa, Artificial Sequence
  Description of Artificial Sequence Synthetic peptide
  MOD_RES (2): Sulfated Tyr
AY

SEQ ID NO: 198 - protein, 2 aa, Artificial Sequence
  Description of Artificial Sequence Synthetic peptide
AY

SEQ ID NO: 199 - protein, 1 aa, Artificial Sequence
  Description of Artificial Sequence Synthetic peptide
  MOD_RES (1): Sulfated Tyr
Y

SEQ ID NO: 200 - protein, 1 aa, Artificial Sequence
  Description of Artificial Sequence Synthetic peptide
Y

SEQ ID NO: 201 - protein, 1 aa, Artificial Sequence
  Description of Artificial Sequence Synthetic peptide
  MOD_RES (1): Sulfated Tyr
Y

SEQ ID NO: 202 - protein, 1 aa, Artificial Sequence
  Description of Artificial Sequence Synthetic peptide
Y

SEQ ID NO: 203 - protein, 5 aa, Artificial Sequence
  Description of Artificial Sequence Synthetic peptide
  MOD_RES (3): Sulfated Tyr
FDYWN

SEQ ID NO: 204 - protein, 5 aa, Artificial Sequence
  Description of Artificial Sequence Synthetic peptide
FDYWN

SEQ ID NO: 205 - protein, 5 aa, Artificial Sequence
  Description of Artificial Sequence Synthetic peptide
  MOD_RES (3): Sulfated Tyr
MMYQW

SEQ ID NO: 206 - protein, 5 aa, Artificial Sequence
  Description of Artificial Sequence Synthetic peptide
MMYQW

SEQ ID NO: 207 - protein, 5 aa, Artificial Sequence
  Description of Artificial Sequence Synthetic peptide
  MOD_RES (3): Sulfated Tyr
YMYLN

SEQ ID NO: 208 - protein, 5 aa, Artificial Sequence
  Description of Artificial Sequence Synthetic peptide
YMYLN

SEQ ID NO: 209 - protein, 5 aa, Artificial Sequence
  Description of Artificial Sequence Synthetic peptide
  MOD_RES (3): Sulfated Tyr
LEYFK

SEQ ID NO: 210 - protein, 5 aa, Artificial Sequence
  Description of Artificial Sequence Synthetic peptide
LEYFK

SEQ ID NO: 211 - protein, 5 aa, Artificial Sequence
  Description of Artificial Sequence Synthetic peptide
  MOD_RES (3): Sulfated Tyr
LIYDY

SEQ ID NO: 212 - protein, 5 aa, Artificial Sequence
  Description of Artificial Sequence Synthetic peptide
LIYDY

SEQ ID NO: 213 - protein, 5 aa, Artificial Sequence
  Description of Artificial Sequence Synthetic peptide
  MOD_RES (3): Sulfated Tyr
KPYYE

SEQ ID NO: 214 - protein, 5 aa, Artificial Sequence
  Description of Artificial Sequence Synthetic peptide
KPYYE

SEQ ID NO: 215 - protein, 5 aa, Artificial Sequence
  Description of Artificial Sequence Synthetic peptide
  MOD_RES (3): Sulfated Tyr
QWYFR

SEQ ID NO: 216 - protein, 5 aa, Artificial Sequence
  Description of Artificial Sequence Synthetic peptide
QWYFR

SEQ ID NO: 217 - protein, 5 aa, Artificial Sequence
  Description of Artificial Sequence Synthetic peptide
  MOD_RES (3): Sulfated Tyr
GKYAK

SEQ ID NO: 218 - protein, 5 aa, Artificial Sequence
  Description of Artificial Sequence Synthetic peptide
GKYAK

SEQ ID NO: 219 - protein, 5 aa, Artificial Sequence
  Description of Artificial Sequence Synthetic peptide
  MOD_RES (3): Sulfated Tyr
NVYET

SEQ ID NO: 220 - protein, 5 aa, Artificial Sequence
  Description of Artificial Sequence Synthetic peptide
NVYET

SEQ ID NO: 221 - protein, 5 aa, Artificial Sequence
  Description of Artificial Sequence Synthetic peptide
  MOD_RES (3): Sulfated Tyr
RFYRN

SEQ ID NO: 222 - protein, 5 aa, Artificial Sequence
  Description of Artificial Sequence Synthetic peptide
RFYRN

SEQ ID NO: 223 - protein, 5 aa, Artificial Sequence
  Description of Artificial Sequence Synthetic peptide
  MOD_RES (3): Sulfated Tyr
FFYTN

SEQ ID NO: 224 - protein, 5 aa, Artificial Sequence
  Description of Artificial Sequence Synthetic peptide
FFYTN

SEQ ID NO: 225 - protein, 5 aa, Artificial Sequence
  Description of Artificial Sequence Synthetic peptide
  MOD_RES (3): Sulfated Tyr
EIYLD

SEQ ID NO: 226 - protein, 5 aa, Artificial Sequence
  Description of Artificial Sequence Synthetic peptide
EIYLD

SEQ ID NO: 227 - protein, 5 aa, Artificial Sequence
  Description of Artificial Sequence Synthetic peptide
  MOD_RES (3): Sulfated Tyr
MYYAF

SEQ ID NO: 228 - protein, 5 aa, Artificial Sequence
  Description of Artificial Sequence Synthetic peptide
MYYAF

SEQ ID NO: 229 - protein, 5 aa, Artificial Sequence
  Description of Artificial Sequence Synthetic peptide
  MOD_RES (3): Sulfated Tyr
NDYSA

SEQ ID NO: 230 - protein, 5 aa, Artificial Sequence
  Description of Artificial Sequence Synthetic peptide
NDYSA

SEQ ID NO: 231 - protein, 5 aa, Artificial Sequence
  Description of Artificial Sequence Synthetic peptide
  MOD_RES (3): Sulfated Tyr
DDYFF

SEQ ID NO: 232 - protein, 5 aa, Artificial Sequence
  Description of Artificial Sequence Synthetic peptide
DDYFF

SEQ ID NO: 233 - protein, 5 aa, Artificial Sequence
  Description of Artificial Sequence Synthetic peptide
  MOD_RES (3): Sulfated Tyr
ASYRH

SEQ ID NO: 234 - protein, 5 aa, Artificial Sequence
  Description of Artificial Sequence Synthetic peptide
ASYRH

SEQ ID NO: 235 - protein, 5 aa, Artificial Sequence
  Description of Artificial Sequence Synthetic peptide
  MOD_RES (3): Sulfated Tyr
VRYFQ

SEQ ID NO: 236 - protein, 5 aa, Artificial Sequence
  Description of Artificial Sequence Synthetic peptide
VRYFQ

SEQ ID NO: 237 - protein, 5 aa, Artificial Sequence
  Description of Artificial Sequence Synthetic peptide
  MOD_RES (3): Sulfated Tyr
QIYKV

SEQ ID NO: 238 - protein, 5 aa, Artificial Sequence
  Description of Artificial Sequence Synthetic peptide
QIYKV

SEQ ID NO: 239 - protein, 5 aa, Artificial Sequence
  Description of Artificial Sequence Synthetic peptide
  MOD_RES (3): Sulfated Tyr
PPYQD

SEQ ID NO: 240 - protein, 5 aa, Artificial Sequence
  Description of Artificial Sequence Synthetic peptide
PPYQD

SEQ ID NO: 241 - protein, 5 aa, Artificial Sequence
  Description of Artificial Sequence Synthetic peptide
  MOD_RES (3): Sulfated Tyr
IFYLI

SEQ ID NO: 242 - protein, 5 aa, Artificial Sequence
  Description of Artificial Sequence Synthetic peptide
IFYLI

SEQ ID NO: 243 - protein, 5 aa, Artificial Sequence
  Description of Artificial Sequence Synthetic peptide
  MOD_RES (3): Sulfated Tyr
KYYEL

SEQ ID NO: 244 - protein, 5 aa, Artificial Sequence
  Description of Artificial Sequence Synthetic peptide
KYYEL

SEQ ID NO: 245 - protein, 5 aa, Artificial Sequence
  Description of Artificial Sequence Synthetic peptide
  MOD_RES (3): Sulfated Tyr
FIYNY

SEQ ID NO: 246 - protein, 5 aa, Artificial Sequence
  Description of Artificial Sequence Synthetic peptide
FIYNY

SEQ ID NO: 247 - protein, 5 aa, Artificial Sequence
  Description of Artificial Sequence Synthetic peptide
  MOD_RES (3): Sulfated Tyr
TFYDK

SEQ ID NO: 248 - protein, 5 aa, Artificial Sequence
  Description of Artificial Sequence Synthetic peptide
TFYDK

SEQ ID NO: 249 - protein, 5 aa, Artificial Sequence
  Description of Artificial Sequence Synthetic peptide
  MOD_RES (3): Sulfated Tyr
QKYSW

SEQ ID NO: 250 - protein, 5 aa, Artificial Sequence
  Description of Artificial Sequence Synthetic peptide
QKYSW

SEQ ID NO: 251 - protein, 5 aa, Artificial Sequence
  Description of Artificial Sequence Synthetic peptide
  MOD_RES (3): Sulfated Tyr
QTYVA

SEQ ID NO: 252 - protein, 5 aa, Artificial Sequence
  Description of Artificial Sequence Synthetic peptide
QTYVA

SEQ ID NO: 253 - protein, 5 aa, Artificial Sequence
  Description of Artificial Sequence Synthetic peptide
  MOD_RES (3): Sulfated Tyr
KVYTT

SEQ ID NO: 254 - protein, 5 aa, Artificial Sequence
  Description of Artificial Sequence Synthetic peptide
KVYTT

SEQ ID NO: 255 - protein, 5 aa, Artificial Sequence
  Description of Artificial Sequence Synthetic peptide
  MOD_RES (3): Sulfated Tyr
GQYNM

SEQ ID NO: 256 - protein, 5 aa, Artificial Sequence
  Description of Artificial Sequence Synthetic peptide
GQYNM

SEQ ID NO: 257 - protein, 5 aa, Artificial Sequence
  Description of Artificial Sequence Synthetic peptide
  MOD_RES (3): Sulfated Tyr
WHYLV

SEQ ID NO: 258 - protein, 5 aa, Artificial Sequence
  Description of Artificial Sequence Synthetic peptide
WHYLV

SEQ ID NO: 259 - protein, 5 aa, Artificial Sequence
  Description of Artificial Sequence Synthetic peptide
  MOD_RES (3): Sulfated Tyr
WFYMA

SEQ ID NO: 260 - protein, 5 aa, Artificial Sequence
  Description of Artificial Sequence Synthetic peptide
WFYMA

SEQ ID NO: 261 - protein, 5 aa, Artificial Sequence
  Description of Artificial Sequence Synthetic peptide
  MOD_RES (3): Sulfated Tyr
GWYKL

SEQ ID NO: 262 - protein, 5 aa, Artificial Sequence
  Description of Artificial Sequence Synthetic peptide
GWYKL

SEQ ID NO: 263 - protein, 5 aa, Artificial Sequence
  Description of Artificial Sequence Synthetic peptide
  MOD_RES (3): Sulfated Tyr
QVYKW

SEQ ID NO: 264 - protein, 5 aa, Artificial Sequence
  Description of Artificial Sequence Synthetic peptide
QVYKW

SEQ ID NO: 265 - protein, 5 aa, Artificial Sequence
  Description of Artificial Sequence Synthetic peptide
  MOD_RES (3): Sulfated Tyr
VWYEM

SEQ ID NO: 266 - protein, 5 aa, Artificial Sequence
  Description of Artificial Sequence Synthetic peptide
VWYEM

SEQ ID NO: 267 - protein, 5 aa, Artificial Sequence
  Description of Artificial Sequence Synthetic peptide
  MOD_RES (3): Sulfated Tyr
NHYSM

SEQ ID NO: 268 - protein, 5 aa, Artificial Sequence
  Description of Artificial Sequence Synthetic peptide
NHYSM

SEQ ID NO: 269 - protein, 5 aa, Artificial Sequence
  Description of Artificial Sequence Synthetic peptide
  MOD_RES (3): Sulfated Tyr
SSYQG

SEQ ID NO: 270 - protein, 5 aa, Artificial Sequence
  Description of Artificial Sequence Synthetic peptide
SSYQG

SEQ ID NO: 271 - protein, 5 aa, Artificial Sequence
  Description of Artificial Sequence Synthetic peptide
  MOD_RES (3): Sulfated Tyr
KDYEP

SEQ ID NO: 272 - protein, 5 aa, Artificial Sequence
  Description of Artificial Sequence Synthetic peptide
KDYEP

SEQ ID NO: 273 - protein, 5 aa, Artificial Sequence
  Description of Artificial Sequence Synthetic peptide
  MOD_RES (3): Sulfated Tyr
HFYWF

SEQ ID NO: 274 - protein, 5 aa, Artificial Sequence
  Description of Artificial Sequence Synthetic peptide
HFYWF

SEQ ID NO: 275 - protein, 5 aa, Artificial Sequence
  Description of Artificial Sequence Synthetic peptide
  MOD_RES (3): Sulfated Tyr
ISYVT

SEQ ID NO: 276 - protein, 5 aa, Artificial Sequence
  Description of Artificial Sequence Synthetic peptide
ISYVT

SEQ ID NO: 277 - protein, 5 aa, Artificial Sequence
  Description of Artificial Sequence Synthetic peptide
  MOD_RES (3): Sulfated Tyr
YRYGL

SEQ ID NO: 278 - protein, 5 aa, Artificial Sequence
  Description of Artificial Sequence Synthetic peptide
YRYGL

SEQ ID NO: 279 - protein, 5 aa, Artificial Sequence
  Description of Artificial Sequence Synthetic peptide
  MOD_RES (3): Sulfated Tyr
SHYWA

SEQ ID NO: 280 - protein, 5 aa, Artificial Sequence
  Description of Artificial Sequence Synthetic peptide
SHYWA

SEQ ID NO: 281 - protein, 5 aa, Artificial Sequence
  Description of Artificial Sequence Synthetic peptide
  MOD_RES (3): Sulfated Tyr
KQYEY

SEQ ID NO: 282 - protein, 5 aa, Artificial Sequence
  Description of Artificial Sequence Synthetic peptide
KQYEY

SEQ ID NO: 283 - protein, 5 aa, Artificial Sequence
  Description of Artificial Sequence Synthetic peptide
  MOD_RES (3): Sulfated Tyr
PFYKS

SEQ ID NO: 284 - protein, 5 aa, Artificial Sequence
  Description of Artificial Sequence Synthetic peptide
PFYKS

SEQ ID NO: 285 - protein, 5 aa, Artificial Sequence
  Description of Artificial Sequence Synthetic peptide
  MOD_RES (3): Sulfated Tyr
PAYHN

SEQ ID NO: 286 - protein, 5 aa, Artificial Sequence
  Description of Artificial Sequence Synthetic peptide
PAYHN

SEQ ID NO: 287 - protein, 5 aa, Artificial Sequence
  Description of Artificial Sequence Synthetic peptide
  MOD_RES (3): Sulfated Tyr
HSYLN

SEQ ID NO: 288 - protein, 5 aa, Artificial Sequence
  Description of Artificial Sequence Synthetic peptide
HSYLN

SEQ ID NO: 289 - protein, 5 aa, Artificial Sequence
  Description of Artificial Sequence Synthetic peptide
  MOD_RES (3): Sulfated Tyr
GRYMW

SEQ ID NO: 290 - protein, 5 aa, Artificial Sequence
  Description of Artificial Sequence Synthetic peptide
GRYMW

SEQ ID NO: 291 - protein, 5 aa, Artificial Sequence
  Description of Artificial Sequence Synthetic peptide
  MOD_RES (3): Sulfated Tyr
KIYFT

SEQ ID NO: 292 - protein, 5 aa, Artificial Sequence
  Description of Artificial Sequence Synthetic peptide
KIYFT

SEQ ID NO: 293 - protein, 5 aa, Artificial Sequence
  Description of Artificial Sequence Synthetic peptide
  MOD_RES (3): Sulfated Tyr
NNYFE

SEQ ID NO: 294 - protein, 5 aa, Artificial Sequence
  Description of Artificial Sequence Synthetic peptide
NNYFE

SEQ ID NO: 295 - protein, 5 aa, Artificial Sequence
  Description of Artificial Sequence Synthetic peptide
  MOD_RES (3): Sulfated Tyr
SMYPG

SEQ ID NO: 296 - protein, 5 aa, Artificial Sequence
  Description of Artificial Sequence Synthetic peptide
SMYPG

SEQ ID NO: 297 - protein, 5 aa, Artificial Sequence
  Description of Artificial Sequence Synthetic peptide
  MOD_RES (3): Sulfated Tyr
MKYGF

SEQ ID NO: 298 - protein, 5 aa, Artificial Sequence
  Description of Artificial Sequence Synthetic peptide
MKYGF

SEQ ID NO: 299 - protein, 5 aa, Artificial Sequence
  Description of Artificial Sequence Synthetic peptide
  MOD_RES (3): Sulfated Tyr
HDYTA

SEQ ID NO: 300 - protein, 5 aa, Artificial Sequence
  Description of Artificial Sequence Synthetic peptide
HDYTA

SEQ ID NO: 301 - protein, 5 aa, Artificial Sequence
  Description of Artificial Sequence Synthetic peptide
  MOD_RES (3): Sulfated Tyr
QHYIY

SEQ ID NO: 302 - protein, 5 aa, Artificial Sequence
  Description of Artificial Sequence Synthetic peptide
QHYIY

SEQ ID NO: 303 - protein, 5 aa, Artificial Sequence
  Description of Artificial Sequence Synthetic peptide
  MOD_RES (3): Sulfated Tyr
QFYEW

SEQ ID NO: 304 - protein, 5 aa, Artificial Sequence
  Description of Artificial Sequence Synthetic peptide
QFYEW

SEQ ID NO: 305 - protein, 5 aa, Artificial Sequence
  Description of Artificial Sequence Synthetic peptide
  MOD_RES (3): Sulfated Tyr
AVYPP

SEQ ID NO: 306 - protein, 5 aa, Artificial Sequence
  Description of Artificial Sequence Synthetic peptide
AVYPP

SEQ ID NO: 307 - protein, 5 aa, Artificial Sequence
  Description of Artificial Sequence Synthetic peptide
  MOD_RES (3): Sulfated Tyr
YRYKW

SEQ ID NO: 308 - protein, 5 aa, Artificial Sequence
  Description of Artificial Sequence Synthetic peptide
YRYKW

SEQ ID NO: 309 - protein, 5 aa, Artificial Sequence
  Description of Artificial Sequence Synthetic peptide
  MOD_RES (3): Sulfated Tyr
IQYQK

SEQ ID NO: 310 - protein, 5 aa, Artificial Sequence
  Description of Artificial Sequence Synthetic peptide
IQYQK

SEQ ID NO: 311 - protein, 5 aa, Artificial Sequence
  Description of Artificial Sequence Synthetic peptide
  MOD_RES (3): Sulfated Tyr
PIYWD

SEQ ID NO: 312 - protein, 5 aa, Artificial Sequence
  Description of Artificial Sequence Synthetic peptide
PIYWD

SEQ ID NO: 313 - protein, 5 aa, Artificial Sequence
  Description of Artificial Sequence Synthetic peptide
  MOD_RES (3): Sulfated Tyr
KAYGL

SEQ ID NO: 314 - protein, 5 aa, Artificial Sequence
  Description of Artificial Sequence Synthetic peptide
KAYGL

SEQ ID NO: 315 - protein, 5 aa, Artificial Sequence
  Description of Artificial Sequence Synthetic peptide
  MOD_RES (3): Sulfated Tyr
DHYRA

SEQ ID NO: 316 - protein, 5 aa, Artificial Sequence
  Description of Artificial Sequence Synthetic peptide
DHYRA

SEQ ID NO: 317 - protein, 5 aa, Artificial Sequence
  Description of Artificial Sequence Synthetic peptide
  MOD_RES (3): Sulfated Tyr
RSYVA

SEQ ID NO: 318 - protein, 5 aa, Artificial Sequence
  Description of Artificial Sequence Synthetic peptide
RSYVA

SEQ ID NO: 319 - protein, 5 aa, Artificial Sequence
  Description of Artificial Sequence Synthetic peptide
  MOD_RES (3): Sulfated Tyr
INYLA

SEQ ID NO: 320 - protein, 5 aa, Artificial Sequence
  Description of Artificial Sequence Synthetic peptide
INYLA

SEQ ID NO: 321 - protein, 5 aa, Artificial Sequence
  Description of Artificial Sequence Synthetic peptide
  MOD_RES (3): Sulfated Tyr
TFYIF

SEQ ID NO: 322 - protein, 5 aa, Artificial Sequence
  Description of Artificial Sequence Synthetic peptide
TFYIF

SEQ ID NO: 323 - protein, 5 aa, Artificial Sequence
  Description of Artificial Sequence Synthetic peptide
  MOD_RES (3): Sulfated Tyr
HIYSR

SEQ ID NO: 324 - protein, 5 aa, Artificial Sequence
  Description of Artificial Sequence Synthetic peptide
HIYSR

SEQ ID NO: 325 - protein, 5 aa, Artificial Sequence
  Description of Artificial Sequence Synthetic peptide
  MOD_RES (3): Sulfated Tyr
EIYHS

SEQ ID NO: 326 - protein, 5 aa, Artificial Sequence
  Description of Artificial Sequence Synthetic peptide
EIYHS

SEQ ID NO: 327 - protein, 5 aa, Artificial Sequence
  Description of Artificial Sequence Synthetic peptide
  MOD_RES (3): Sulfated Tyr
QQYQP

SEQ ID NO: 328 - protein, 5 aa, Artificial Sequence
  Description of Artificial Sequence Synthetic peptide
QQYQP

SEQ ID NO: 329 - protein, 5 aa, Artificial Sequence
  Description of Artificial Sequence Synthetic peptide
  MOD_RES (3): Sulfated Tyr
MFYEA

SEQ ID NO: 330 - protein, 5 aa, Artificial Sequence
  Description of Artificial Sequence Synthetic peptide
MFYEA

SEQ ID NO: 331 - protein, 5 aa, Artificial Sequence
  Description of Artificial Sequence Synthetic peptide
  MOD_RES (3): Sulfated Tyr
EVYLE

SEQ ID NO: 332 - protein, 5 aa, Artificial Sequence
  Description of Artificial Sequence Synthetic peptide
EVYLE

SEQ ID NO: 333 - protein, 5 aa, Artificial Sequence
  Description of Artificial Sequence Synthetic peptide
  MOD_RES (3): Sulfated Tyr
DAYAN

SEQ ID NO: 334 - protein, 5 aa, Artificial Sequence
  Description of Artificial Sequence Synthetic peptide
DAYAN

SEQ ID NO: 335 - protein, 452 aa, Homo sapiens
QVQLQESGAEVKKPGASVKVSCKSSGYTFTAYYMHWLRQAPGQGLEWMGWINPNSGGTNYAQKFQGRVTLTRDTSISTAYMELNSLTSDDTAMYYCARGGPRVSSRPGIGYSDSWGKGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 336 - protein, 215 aa, Homo sapiens
QTVVLQEPSLTVSPGGTVTLTCASRIGAVTSGHYANWFQQKPGQAPRALIYRTNNKQSWTPARFSGSLLGDRAALTLSGAQPEDEADYYCLLYYGGSWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 337 - protein, 447 aa, Homo sapiens
EVQLVESGGDLVQPGESLGLSCVGSEFNFGSYGMTWVRQAPGKGLEWVSSISSAGKTFYADSVKGRFTISRDNSKNTVFLQMNNLRVEDTAVYYCAKGRGHSYGRPLASWGRGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 338 - protein, 221 aa, Homo sapiens
  MOD_RES (14): Variable amino acid
QAVLTQPSSLSASPGASASLTCTLRSGIDVGPHRIYWFQQKPGSTPQYLLRYKSDSDTQQGSGVPSRFSGSKDASANAGILLISGLQSEDEADYYCMIWHSSAWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 339 - protein, 14 aa, Artificial Sequence
  Description of Artificial Sequence Synthetic peptide
  MOD_RES (1): biotin-beta-Ala
AKKVVALYDYMPMN

SEQ ID NO: 340 - protein, 15 aa, Artificial Sequence
  Description of Artificial Sequence Synthetic linker peptide
GGGGSGGGGSGGGGS